(19) United States
(12) Patent Application Publication
(10) Pub. No.: US 2022/0389099 A1
(43) Pub. Date: Dec. 8, 2022

(54) METHODS FOR TREATING LEUKEMIA

(71) Applicant: AMGEN INC., Thousand Oaks, CA (US)

(72) Inventors: Suresh AGARWAL, San Francisco, CA (US); Sophia KHALDOYANIDI, San Diego, CA (US); Dirk NAGORSEN, Thousand Oaks, CA (US); Vijay UPRETI, San Francisco, CA (US)

(73) Assignee: AMGEN INC., Thousand Oaks, CA (US)

(21) Appl. No.: 17/774,125

(22) PCT Filed: Nov. 3, 2020
(86) PCT No.: PCT/US20/58720

§ 371 Date: May 3, 2022

Related U.S. Application Data
(60) Provisional application No. 63/024,407, filed on May 13, 2020.
(60) Provisional application No. 62/930,433, filed on Nov. 4, 2019.

(51) Int. Cl. C07K 16/28; A61K 31/573; A61K 39/395; A61P 35/02

(52) U.S. Cl. CPC C07K 16/2803; C07K 16/2809; A61K 31/573; A61K 39/3955; A61P 35/02; C07K 2317/31; A61K 2039/545

(57) ABSTRACT

The present invention relates to treatment methods for leukemia using bispecific antibody constructs that specifically bind to human CD33 and human CD3. In particular, the present invention relates to methods for treating myeloid leukemia, including relapsed/refractory myeloid leukemia, in a patient in need thereof comprising administering to the patient at least one initiation cycle and at least one maintenance cycle of an anti-CD33×anti-CD3 bispecific antibody construct, wherein each initiation cycle and maintenance cycle comprises administering the bispecific antibody construct according to specific dosage regimens. Pharmaceutical compositions comprising the bispecific antibody constructs for use in the methods are also disclosed.

### CROSS-REFERENCE TO RELATED APPLICATIONS

This application claims the benefit of U.S. Provisional Application No. 62/930,433, filed Nov. 4, 2019, and U.S. Provisional Application No. 63/024,407, filed May 13, 2020, both of which are hereby incorporated by reference in their entireties.

### DESCRIPTION OF THE TEXT FILE SUBMITTED ELECTRONICALLY

The present application contains a Sequence Listing, which has been submitted electronically in ASCII format and is hereby incorporated by reference in its entirety. The computer readable format copy of the Sequence Listing, which was created on Oct. 27, 2020, is named A-2426-WO-PCT_ST25 and is 270 kilobytes in size.

### FIELD OF THE INVENTION

The present invention relates to the fields of immuno-oncology and biopharmaceuticals. In particular, the invention relates to methods of treating myeloid leukemia by administering a bispecific antibody construct that specifically binds to CD33 and CD3 in at least one initiation cycle and one maintenance cycle, wherein the initiation cycle and maintenance cycle each comprises administering the bispecific antibody construct according to specific dosing regimens.

### BACKGROUND OF THE INVENTION

Acute myeloid leukemia (AML) is the most common form of acute leukemia in adults in the United States (US), with a rising incidence possibly due to an aging population, increased environmental exposure, and an increase in the population of cancer survivors previously exposed to chemotherapy and therapeutic radiation. In 2016, an estimated 19,950 new cases of AML were expected in the US with approximately 10,430 deaths occurring from this disease (American Cancer Society, 2016).

Outcomes for most patients with AML remain poor (Burnett et al., J. Clin. Oncol., Vol. 29:487-494, 2011). In particular, relapsed disease is associated with unsatisfactory outcomes in the majority of patients (Ravandi, Best Pract. Res. Clin. Haematol., Vol. 26:253-259, 2013). Although the majority of patients with AML initially achieve complete remission (CR), over 60% will eventually relapse after a variable period of remission. Using the traditional cytotoxic chemotherapy regimens, the likelihood of achieving a second CR is low especially if the first CR was short in duration (Estey et al., Blood, Vol. 88:756, 1996). This is particularly true for patients who have primary refractory disease and have never achieved a morphological response. For example, patients with AML refractory to one course of a high dose cytarabine (HiDAC)-containing regimen have a median overall survival of only 3.8 months (Ravandi et al., Blood, Vol. 116:5818-5823, 2010). Patients whose initial CR duration is more than 1 year have been traditionally treated with HiDAC-containing salvage regimens but only a minority achieve a second CR, and many are not candidates for a potentially curative allogeneic hematopoietic stem cell transplant (Estey, Leukemia, Vol. 14:476-479, 2000). Apart from duration of first CR, other predictors of outcome of first relapse include age, cytogenetics, and whether the patient received an allogeneic hematopoietic stem cell transplant in first CR (Breems et al., J. Clin. Oncol., Vol. 23:1969-1978, 2005). However, in the study reported by Breems et al. (2005), only a minority of patients with AML in first relapse had a successful long-term outcome and the long-term prognosis of the majority of patients with relapsed or refractory AML remains unfavorable.

Cluster of differentiation 33 (CD33) provides a useful target antigen for the treatment of patients with AML, as it is expressed on the cell surface of more than 80% of leukemia isolates from patients with AML with a very high average antigen density (Tanimoto et al., Leukemia, Vol. 3:339-348, 1989; Scheinberg et al., Leukemia, Vol. 3:440-445, 1989). It is not expressed on tissues other than the hematopoietic system and whether it is expressed by the normal multipotent hematopoietic stem cells has been a point of debate (Taussig et al., Blood, Vol. 106:4086-4092, 2005; Pearce et al., Cell Cycle, Vol. 5:271-273, 2006). The prototype unconjugated monoclonal antibody against CD33, HuM195, has been shown to rapidly internalize into target cells upon binding the CD33 antigen (Caron et al., Cancer, Vol. 73:1049-1056, 1994). Calicheamicin, a potent anti-tumor antibiotic, was conjugated to the CD33 antibody and the resultant gemtuzumab ozogamicin (GO) was effective in producing responses in about 30% of older patients (>60 years) with AML in first relapse (Sievers et al., Blood, Vol. 93:3678-3684, 1999). This led to the accelerated approval of the drug as a single agent for the same population. However, failure to demonstrate a clinical benefit in the confirmatory trial, together with concern about an increased risk of veno-occlusive disease led to its voluntary withdrawal from the market by the manufacturers (Ravandi, J. Clin. Oncol., Vol. 29:349-351, 2011). However, its efficacy, when combined in low doses with chemotherapy, has been demonstrated in a number of randomized European trials, demonstrating that CD33 is a significant target for therapeutic drug development in AML (Castaigne et al., Lancet, Vol. 379:1508-1516, 2012; Ravandi et al., J. Clin. Oncol., Vol. 30:3921-3923, 2012; Burnett et al., J. Clin. Oncol., Vol. 29:369-377, 2011).

Thus, there remains a need for novel effective therapies, such as CD33-targeting therapies, for the treatment of myeloid leukemia and other myeloid malignancies, particularly for relapsed or refractory AML patients, who have a poor long-term prognosis.

### SUMMARY OF THE INVENTION

The present invention is based, in part, on the identification of therapeutic regimens of a an anti-CD33×anti-CD3 bispecific antibody construct for effectively treating myeloid leukemia, particularly relapsed or refractory acute myeloid leukemia. Accordingly, in one embodiment, the present invention provides a method for treating myeloid leukemia in a patient in need thereof comprising administering to the patient at least one initiation cycle and at least one maintenance cycle of a bispecific antibody construct that specifically binds to CD33 and CD3.

In certain embodiments, the initiation cycle comprises administering an anti-CD33×anti-CD3 bispecific antibody construct in one or more doses at an interval of 1 day to 4 days for a first period of time, such as from about 5 days to about 30 days or from about 7 days to about 14 days. In some embodiments, the initiation cycle comprises administering the anti-CD33×anti-CD3 bispecific antibody construct daily (e.g. once per day) for a first period of time, such as once per day for 7 days or once per day for 14 days. In other embodiments, the initiation cycle comprises administering the anti-CD33×anti-CD3 bispecific antibody construct once every other day (e.g. Q2D) or once every three days (e.g. Q3D) for a first period of time (e.g. 14 days). In still other embodiments, the initiation cycle comprises administering the anti-CD33×anti-CD3 bispecific antibody construct once every four days (e.g. Q4D) for 7 days or 14 days.

The one or more doses of an anti-CD33×anti-CD3 bispecific antibody construct administered during the initiation cycle can be from about 18 μg to about 480 μg at each dosing interval, for example from about 36 μg to about 480 μg, from about 72 μg to about 480 μg, from about 110 μg to about 360 μg, from about 18 μg to about 240 μg, or from about 100 μg to about 180 μg at each dosing interval. In certain embodiments, the dose of the anti-CD33×anti-CD3 bispecific antibody construct administered during the initiation cycle is the same at each interval (e.g. a fixed dose for the entire cycle). In alternative embodiments, the dose of the bispecific antibody construct administered during the initiation cycle changes from one dosing interval to subsequent dosing intervals. For instance, in one embodiment, the dose of the bispecific antibody construct administered during the initiation cycle increases at least once at one or more intervals during the cycle (e.g. step dosing).

In some embodiments in which a step dosing regimen is employed during the initiation cycle, the initiation cycle comprises administering the anti-CD33×anti-CD3 bispecific antibody construct at a first dose for one or more intervals and subsequently administering the anti-CD33×anti-CD3 bispecific antibody construct at a second dose for one or more intervals, wherein the second dose is greater than the first dose. One or more dose increases or dosage steps can be used, for example, 2, 3, 4, 5, 6, or 7 or more dosage steps. For instance, in such embodiments where two dosage steps are used (i.e. three different doses are administered), the initiation cycle comprises administering the anti-CD33×anti-CD3 bispecific antibody construct at a first dose for one or more intervals, subsequently administering the bispecific antibody construct at a second dose for one or more intervals, and subsequently administering the bispecific antibody construct at a third dose for one or more intervals, wherein the third dose is greater than the second dose and the second dose is greater than the first dose. In some embodiments three dosage steps are used (i.e. four different doses are administered), in which case the initiation cycle may comprise administering the anti-CD33×anti-CD3 bispecific antibody construct at a first dose for one or more intervals, subsequently administering the bispecific antibody construct at a second dose for one or more intervals, subsequently administering the bispecific antibody construct at a third dose for one or more intervals, and subsequently administering the bispecific antibody construct at a fourth dose for one or more intervals, wherein the fourth dose is greater than the third dose, the third dose is greater than the second dose, and the second dose is greater than the first dose. In certain embodiments four dosage steps are used (i.e. five different doses are administered), in which case the initiation cycle may comprise administering the anti-CD33×anti-CD3 bispecific antibody construct at a first dose for one or more intervals, subsequently administering the bispecific antibody construct at a second dose for one or more intervals, subsequently administering the bispecific antibody construct at a third dose for one or more intervals, subsequently administering the bispecific antibody construct at a fourth dose for one or more intervals, and subsequently administering the bispecific antibody construct at a fifth dose for one or more intervals, wherein the fifth dose is greater than the fourth dose, the fourth dose is greater than the third dose, the third dose is greater than the second dose, and the second dose is greater than the first dose. In certain other embodiments five dosage steps are used (i.e. six different doses are administered), in which case the initiation cycle may comprise administering the anti-CD33×anti-CD3 bispecific antibody construct at a first dose for one or more intervals, subsequently administering the bispecific antibody construct at a second dose for one or more intervals, subsequently administering the bispecific antibody construct at a third dose for one or more intervals, subsequently administering the bispecific antibody construct at a fourth dose for one or more intervals, subsequently administering the bispecific antibody construct at a fifth dose for one or more intervals, and subsequently administering the bispecific antibody construct at a sixth dose for one or more intervals, wherein the sixth dose is greater than the fifth dose, the fifth dose is greater than the fourth dose, the fourth dose is greater than the third dose, the third dose is greater than the second dose, and the second dose is greater than the first dose. The dosing intervals for the step dosing regimens employed during the initiation cycle can be daily, once every two days, once every three days or once every four days. In one particular embodiment, the dosing interval for a step dosing regimen employed during the initiation cycle is daily (e.g. once per day).

The step doses of an anti-CD33×anti-CD3 bispecific antibody construct can be any of the doses within the ranges described herein for administration during the initiation cycle, such as from about 18 μg to about 480 μg, from about 36 μg to about 480 μg, from about 72 μg to about 480 μg, from about 110 μg to about 360 μg, from about 18 μg to about 240 μg, or from about 100 μg to about 180 μg. In certain embodiments, the first dose is from about 18 μg to about 150 μg and the second dose is from about 110 μg to about 240 μg, wherein the second dose is greater than the first dose. In other embodiments, the first dose is from about 18 μg to about 110 μg and the second dose is from about 72 μg to about 160 μg, wherein the second dose is greater than the first dose. In one embodiment, the first dose is about 36 μg and the second dose is about 72 μg. In embodiments where two dosage steps are employed (i.e. three different doses administered during the initiation cycle), the first dose can be from about 18 μg to about 150 μg, the second dose can be from about 110 μg to about 240 μg, and the third dose can be from about 150 μg to about 360 μg, wherein the third dose is greater than the second dose and the second dose is greater than the first dose. In other embodiments where three dosage steps are employed (i.e. four different doses administered during the initiation cycle), the first dose can be from about 18 μg to about 150 μg, the second dose can be from about 110 μg to about 240 μg, the third dose can be from about 150 μg to about 360 μg, and the fourth can be from about 180 μg to about 480 μg, wherein the fourth dose is greater than the third dose, the third dose is greater than the second dose, and the second dose is greater than the first dose. In certain other embodiments where three dosage steps are employed (i.e. four different doses administered during the initiation cycle), the first dose can be from about 18 μg to about 110 μg, the second dose can be from about 36 μg to about 160 μg, the third dose can be from about 72 μg to about 240 μg, and the fourth can be from about 110 μg to about 480 μg, wherein the fourth dose is greater than the third dose, the third dose is greater than the second dose, and the second dose is greater than the first dose. In one particular embodiment, the first dose is about 18 μg, the second dose is about 36 μg, the third dose is about 72 μg, and the fourth dose is about 110 μg.

In some embodiments where four dosage steps are employed (i.e. five different doses administered during the initiation cycle), the first dose can be from about 18 μg to about 110 μg, the second dose can be from about 36 μg to about 160 μg, the third dose can be from about 72 μg to about 240 μg, the fourth dose can be from about 110 μg to about 360 μg, and the fifth dose can be from about 160 μg to about 480 μg, wherein the fifth dose is greater than the fourth dose, the fourth dose is greater than the third dose, the third dose is greater than the second dose, and the second dose is greater than the first dose. In one such embodiment, the first dose is about 18 fig, the second dose is about 36 μg, the third dose is about 72 μg, the fourth dose is about 110 μg, and the fifth dose is about 160 μg. In embodiments in which five dosage steps are employed (i.e. six different doses administered during the initiation cycle), the first dose can be from about 18 μg to about 72 μg, the second dose can be from about 36 μg to about 110 μg, the third dose can be from about 72 μg to about 160 μg, the fourth dose can be from about 110 μg to about 240 μg, the fifth dose can be from about 160 μg to about 360 μg, and the sixth dose can be from about 240 μg to about 480 μg, wherein the sixth dose is greater than the fifth dose, the fifth dose is greater than the fourth dose, the fourth dose is greater than the third dose, the third dose is greater than the second dose, and the second dose is greater than the first dose. In certain such embodiments, the first dose is about 18 μg, the second dose is about 36 μg, the third dose is about 72 μg, the fourth dose is about 110 μg, the fifth dose is about 160 μg, and the sixth dose is about 240 μg.

In certain embodiments of the methods of the invention, the maintenance cycle comprises administering the anti-CD33×anti-CD3 bispecific antibody construct once or twice every 7 days (e.g. once per week or twice per week) for a second period time, such as from about 14 days to about 60 days, from about 15 days to about 30 days, or from about 14 days to about 28 days. According to the methods of the invention, the maintenance cycle is administered after the initiation cycle. In a preferred embodiment, the maintenance cycle is initiated the following day after completing the initiation cycle, for example with no treatment-free periods between the initiation cycle and the maintenance cycle. A patient may receive multiple maintenance cycles, such as 2, 3, 4, 5, 6, 7, 8, 9, 10, 11, or 12 or more maintenance cycles. In some embodiments, maintenance cycles are administered to the patient until the patient responds to treatment, for example achieves a complete remission.

During the maintenance cycle, the dose of anti-CD33×anti-CD3 bispecific antibody construct administered to the patient once per week or twice per week in some embodiments of the methods of the invention can be from about 36 μg to about 480 μg, about 72 μg to about 200 fig, about 72 μg to about 360 μg, about 100 μg to about 180 μg, or about 110 μg to about 240 FIG. 1n certain embodiments, the dose of the bispecific antibody construct administered during the maintenance cycle is the same as the highest dose of the bispecific antibody construct administered during the initiation cycle. In one embodiment, the maintenance cycle comprises administering the dose of the bispecific antibody construct once every 7 days (e.g. once per week) for 14 days or for 28 days. In another embodiment, the maintenance cycle comprises administering the dose of the bispecific antibody construct twice every 7 days (e.g. twice per week) for 14 days or for 28 days.

In some embodiments, the myeloid leukemia to be treated according to the methods of the invention is acute myeloid leukemia. Accordingly, the patient to be treated according to the methods of the invention has or is diagnosed with acute myeloid leukemia. In one embodiment, the patient to be treated according to the methods of the invention has or is diagnosed with relapsed acute myeloid leukemia. In another embodiment, the patient to be treated according to the methods of the invention has or is diagnosed with refractory acute myeloid leukemia. In other embodiments, the myeloid leukemia to be treated according to the methods of the invention is chronic myeloid leukemia. Thus, the patient to be treated according to the methods of the invention has or is diagnosed with chronic myeloid leukemia.

Myeloid leukemia patients to be treated according to the methods of the invention may have received one or more prior therapies for myeloid leukemia. In some embodiments, the patients have received one or more chemotherapy regimens, such as cytarabine-containing regimens. Additionally or alternatively, the patients have received an allogeneic or autologous hematopoietic stem cell transplant. In certain embodiments, the patients to be treated according to the methods of the invention may be refractory to or have relapsed from these prior therapies.

In certain embodiments of the methods described herein, the anti-CD33×anti-CD3 bispecific antibody construct is administered to the patient parenterally, preferably intravenously. The intravenous administration can be an intravenous infusion, such as intravenous infusion of about 30 min to about 3 hours or more preferably of about 30 min to about 90 min. In some embodiments, each of the doses of the bispecific antibody construct administered during the initiation cycle and/or maintenance cycle is administered as an intravenous infusion.

In some embodiments, the methods of the invention may further comprise administering to the patient one or more premedications prior to administration of one or more (or all) doses of the bispecific antibody construct during the initiation cycle and/or maintenance cycle. Premedications can include antihistamines (e.g. diphenhydramine), glucocorticoids (e.g. dexamethasone), and IL6 receptor antagonists (e.g. tocilizumab).

In any embodiments of the methods disclosed herein, the bispecific antibody construct administered to the patient specifically binds to CD33 and CD3, preferably human CD33 and human CD3. Thus, the bispecific antibody construct comprises a first binding domain that specifically binds to CD33 and a second binding domain that specifically binds to CD3. In certain embodiments, the first binding domain specifically binds to human CD33 and the second binding domain specifically binds to human CD3 epsilon. The binding domains can be derived from antibodies or antigen-binding fragments thereof, such as heavy and light chain variable regions. In one embodiment, either or both of the binding domains of the bispecific antibody construct used in the methods of the invention is a single-chain variable fragment (scFv). In some embodiments of the methods described herein, the bispecific antibody constructs further comprise a third domain having one or more immunoglobulin Fc regions. In such embodiments, the third domain can be a single-chain Fc domain.

In certain embodiments, the bispecific antibody construct administered to the patient according to the methods of the invention comprises, in an amino to carboxyl order: (i) a first domain that specifically binds to human CD33, (ii) a second domain that specifically binds to human CD3, and (iii) a third domain comprising two Fc monomers, each monomer comprising an immunoglobulin hinge region, a CH2 domain, and a CH3 domain, wherein said two Fc monomers are fused to each other via a peptide linker. In one embodiment, the first domain comprises a first immunoglobulin heavy chain variable region (VH1) comprising a CDRH1 having the sequence of SEQ ID NO: 10, a CDRH2 having the sequence of SEQ ID NO: 13, and a CDRH3 having the sequence of SEQ ID NO: 14, and a first immunoglobulin light chain variable region (VL1) comprising a CDRL1 having the sequence of SEQ ID NO: 6, a CDRL2 having the sequence of SEQ ID NO: 8, and a CDRL3 having the sequence of SEQ ID NO: 9. In a related embodiment, the second domain comprises a second immunoglobulin heavy chain variable region (VH2) comprising a CDRH1 having the sequence of SEQ ID NO: 38, a CDRH2 having the sequence of SEQ ID NO: 44, and a CDRH3 having the sequence of SEQ ID NO: 49, and a second immunoglobulin light chain variable region (VL2) comprising a CDRL1 having the sequence of SEQ ID NO: 32, a CDRL2 having the sequence of SEQ ID NO: 33, and a CDRL3 having the sequence of SEQ ID NO: 36. In some embodiments, the first domain of the bispecific antibody construct comprises a heavy chain variable region comprising the sequence of SEQ ID NO: 28 and a light chain variable region comprising the sequence of SEQ ID NO: 20. In these and other embodiments, the second domain of the bispecific antibody construct comprises a heavy chain variable region comprising the sequence of SEQ ID NO: 61 and a light chain variable region comprising the sequence of SEQ ID NO: 59.

The bispecific antibody construct administered to patients according to the methods of the invention may comprise (i) a first domain that specifically binds to human CD33 and has the amino acid sequence of SEQ ID NO: 91, (ii) a second domain that specifically binds to human CD3 and has the amino acid sequence of SEQ ID NO: 101, and (iii) a third domain comprising two Fc monomers each having the amino acid sequence of SEQ ID NO: 109, wherein said two Fc monomers are fused to each other via a peptide linker. In related embodiments, the third domain of the bispecific antibody construct comprises the amino acid sequence of SEQ ID NO: 117. In certain embodiments, the bispecific antibody construct used in the methods of the invention is a single chain antibody construct. Thus, any of the single chain antibody constructs described in Table 6 herein are suitable for use in the methods of the invention. In a preferred embodiment, the bispecific antibody construct administered to a patient according to the methods of the invention comprises the amino acid sequence of SEQ ID NO: 125.

The present invention also provides pharmaceutical compositions of anti-CD33×anti-CD3 bispecific antibody constructs for use in the methods described herein. The pharmaceutical compositions can comprise one or more pharmaceutically acceptable diluents, carriers, or excipients, including buffers, surfactants, and stabilizing agents. In certain embodiments, the pharmaceutical compositions comprise an anti-CD33×anti-CD3 bispecific antibody construct, a buffer, a surfactant, and a stabilizing agent. In one embodiment, the pharmaceutical composition comprises an anti-CD33×anti-CD3 bispecific antibody construct (e.g. comprising the amino acid sequence of SEQ ID NO: 125), a glutamate buffer, polysorbate 20 or polysorbate 80, and sucrose, at a pH of about 4.0 to about 4.4. In some embodiments, the pharmaceutical compositions may be lyophilized and reconstituted prior to administration to a patient.

In some embodiments, the present invention also provides kits comprising a pharmaceutical composition disclosed herein and instructions for using the pharmaceutical composition for delivering a therapeutically effective dose, for example, by intravenous infusion for treating myeloid leukemia in a patient in need thereof. In embodiments in which the pharmaceutical composition is provided in a lyophilized or dry powder form, the kit may comprise a diluent and instructions for reconstituting the pharmaceutical composition prior to administration. In certain embodiments, the kits may further comprise one or more vials of intravenous solution stabilizer (IVSS) and instructions for using the IVSS for pre-treatment of IV bags prior to dilution of the pharmaceutical composition for delivery to the patient.

The use of anti-CD33×anti-CD3 bispecific antibody constructs in any of the methods disclosed herein or for preparation of medicaments for administration according to any of the methods disclosed herein is specifically contemplated. For instance, the present invention includes a bispecific antibody construct that specifically binds to CD33 and CD3 for use in a method for treating myeloid leukemia in a patient in need thereof, wherein the method comprises administering to the patient at least one initiation cycle and at least one maintenance cycle of the bispecific antibody construct, wherein the initiation cycle comprises administering the bispecific antibody construct at one or more doses of about 18 μg to about 480 μg at an interval of 1 day to 4 days for a first period of time, wherein the maintenance cycle comprises administering the bispecific antibody construct at a dose of about 36 μg to about 480 μg once or twice every 7 days for a second period of time, and wherein the maintenance cycle is administered after the initiation cycle. The present invention also includes the use of a bispecific antibody construct that specifically binds to CD33 and CD3 for the manufacture of a medicament for the treatment of myeloid leukemia in a patient in need thereof, wherein the treatment comprises administering to the patient at least one initiation cycle and at least one maintenance cycle of the bispecific antibody construct, wherein the initiation cycle comprises administering the bispecific antibody construct at one or more doses of about 18 μg to about 480 μg at an interval of 1 day to 4 days for a first period of time, wherein the maintenance cycle comprises administering the bispecific antibody construct at a dose of about 36 μg to about 480 μg once or twice every 7 days for a second period of time, and wherein the maintenance cycle is administered after the initiation cycle.

### BRIEF DESCRIPTION OF THE DRAWINGS

FIG. 1A is a graph of the relationship between tumor necrosis factor-alpha (TNF-α) serum levels in patients with relapsed/refractory acute myeloid leukemia treated with AMG 673 and grade of cytokine release syndrome (CRS) experienced by the patients. The TNF-α serum levels are plotted as median±interquartile range.

FIG. 1B is a graph of the relationship between interleukin-2 (IL-2) serum levels in patients with relapsed/refractory acute myeloid leukemia treated with AMG 673 and grade of CRS experienced by the patients. The IL-2 serum levels are plotted as median±interquartile range.

FIG. 1C is a graph of the relationship between interleukin-6 (IL-6) serum levels in patients with relapsed/refractory acute myeloid leukemia treated with AMG 673 and grade of CRS experienced by the patients. The IL-6 serum levels are plotted as median±interquartile range.

FIG. 1D is a graph of the relationship between interleukin-10 (IL-10) serum levels in patients with relapsed/refractory acute myeloid leukemia treated with AMG 673 and grade of CRS experienced by the patients. The IL-10 serum levels are plotted as median±interquartile range.

FIG. 2 is a line graph of the serum concentrations of cytokines (interferon-gamma (IN-gamma), IL-10, IL-6, and TNF-alpha) in relapsed/refractory acute myeloid leukemia patients at the indicated time points following administration of AMG 673 at day 1 and day 5 in cycle 1 of treatment. Data is shown for patients in cohorts 8-10, who received a dose of 18 μg, 36 μg, or 72 μg, respectively, at each infusion.

FIG. 3 is a waterfall plot showing percentage change in bone marrow blasts from baseline to best response in patients with relapsed/refractory acute myeloid leukemia treated with AMG 673. *The percentage change in blasts from baseline for this patient was 469. AMG 673 was administered by intravenous infusion on day 1 and day 5 of a 14-day treatment cycle. The dose administered at each infusion was 0.05 μg, 0.15 μg, 0.45 μg, 1.5 μg, 4.5 μg, 7 μg, 9 μg, 18 μg, 36 μg, or 72 μg for cohorts 1-10, respectively.

FIG. 4 shows the serum exposure of AMG 673 during cycle 1 of treatment, as measured by area under the curve (AUC), in relapsed/refractory acute myeloid leukemia patients who either exhibited greater than 20% reduction in bone marrow blasts or less than 20% reduction or no reduction in bone marrow blasts following treatment with AMG 673 across dosing cohorts 1-10.

FIG. 5A shows the change in percentage of CD69+CD8+ T cells as a function of dosing cohort. The dose administered at each infusion was 0.05 μg, 0.15 μg, 0.45 μg, 1.5 μg, 4.5 μg, 7 μg, 9 μg, 18 μg, 36 μg, or 72 μg for cohorts 1-10, respectively.

FIG. 5B shows the fold-change in serum levels of interferon-gamma (IFN-γ) as a function of dosing cohort. The dosing cohorts are the same as those described for FIG. 5A.

FIG. 5C shows the fold-change in serum levels of tumor necrosis factor-alpha (TNF-α) as a function of dosing cohort. The dosing cohorts are the same as those described for FIG. 5A.

### DETAILED DESCRIPTION

Bispecific T cell engager molecules are new immunotherapies being developed for the treatment of various cancers. These molecules typically have at least one binding domain that is specific for a cell-surface antigen expressed on cancer cells and at least another binding domain that is specific for cluster of differentiation 3 (CD3), a subunit of the T cell receptor complex expressed on T cells. Bispecific T cell engager molecules are designed to connect T cells with target cancer cells and potently activate the inherent cytolytic potential of T cells against the target cancer cells. The first generation of bispecific T cell engager molecules (see, e.g., WO 99/54440, WO 2005/040220, and WO 2008/119567) are typically administered by continuous intravenous infusion due to half-lives of less than a day. A second generation of bispecific T cell engager molecules (see, e.g., WO 2013/128027, WO 2014140358, WO 2014/144722, WO 2014/151910, WO 2017/134140) have been designed, at least in part, to increase the serum half-life of the molecules to enable dosing paradigms that do not involve continuous administration.

Because the mechanism of action of bispecific T cell engager molecules involves T cell activation, a potential side effect of these molecules is cytokine release syndrome (CRS). CRS can occur when large numbers of T cells are activated and release inflammatory cytokines. To minimize the effects of cytokine elevation and the development of CRS, bispecific T cell engager molecules can be administered at lower doses or by employing anti-histamines or corticosteroid pre-treatments. In the case of second-generation bispecific T cell engager molecules where the effects of the molecules, including the undesired side effects, may be prolonged due to the longer serum half-life, it is important to develop a dosing strategy that allows the patients to be exposed to efficacious doses as quickly as possible while at the same time limiting or avoiding the side effects associated with rapid cytokine elevation, such as CRS. The present invention addresses this need by providing novel dosing regimens for an anti-CD33×anti-CD3 bispecific antibody construct for the treatment of myeloid leukemia. Accordingly, in one aspect, the present invention provides a method for treating myeloid leukemia in a patient in need thereof comprising administering to the patient a bispecific antibody construct that specifically binds to CD33 and CD3 in at least one initiation cycle and at least one maintenance cycle as described further herein.

Leukemia is a group of cancers that affect the blood and bone marrow. Leukemia usually begins in the bone marrow and results in high numbers of abnormal progenitor cells, which are not fully developed and are called blasts (or myeloblasts) or leukemia cells. Leukemia is primarily characterized as chronic or acute and as lymphocytic or myelogenous. Lymphocytic leukemia refers to abnormal bone marrow progenitor cells that become lymphocytes, whereas myelogenous or myeloid leukemia refers to abnormal bone marrow progenitor cells that mature into cells of myeloid lineage, including red blood cells, some white blood cells (e.g. neutrophils and monocytes), and platelets.

Signs and symptoms of leukemia can vary according to the particular type and stage of leukemia and may include, but are not limited to, flu-like symptoms, such as fever and chills, weakness, fatigue, swollen or bleeding gums, headaches, enlarged liver or spleen, swollen tonsils, bone or joint pain, pinhead-sized red spots on the skin, pale complexion, and weight loss. The presence and type of leukemia is typically diagnosed by one or more tests conducted on a sample (e.g. blood, bone marrow, and/or lymph node sample) from a subject or patient suspected to have or develop leukemia, according to the WHO classification criteria for myeloid neoplasms and acute leukemia (see Table 1 of Arber et al., Blood, Vol. 127: 2391-2405, 2016). A sample can be any biological sample obtained from a human patient and can include body fluids, such as blood, serum, plasma, urine, and saliva, and tissues, such as bone marrow or lymph nodes.

In certain embodiments, the patients to be treated according to the methods of the invention have or are diagnosed with myeloid leukemia (also known as myelogenous leukemia). Blood samples, bone marrow biopsies, and/or bone marrow aspirates obtained from the subject or patient are preferred, in some embodiments, to diagnose the type of myeloid leukemia. In some embodiments, the patients to be treated according to the methods of the invention have or are diagnosed with acute myeloid leukemia (also known as acute myelogenous leukemia). Patients diagnosed with acute myeloid leukemia usually have lower-than-expected levels of red blood cells and platelets in the blood and the presence of leukemic blast cells in the blood and/or bone marrow. Diagnosis of acute myeloid leukemia can be confirmed by 20% or more leukemic blast cells (myeloblasts) in blood or bone marrow, presence of leukemic blast cells in bone marrow having certain recurrent genetic abnormalities (i.e. chromosome changes) associated with acute myeloid leukemia, or presence of leukemic blast cells expressing particular surface proteins, such as CD13 or CD33. Diagnosis can be supplemented by gene-expression profiling, cytogenetics, karyotyping, or immunophenotyping to confirm an initial diagnosis and/or identify a subtype of acute myeloid leukemia. The diagnosis of acute myeloid leukemia is preferably made by a hematopathologist with experience in diagnosing leukemias by, preferably applying the WHO classification of myeloid neoplasms and acute leukemia (see Table 1 of Arber et al., Blood, Vol. 127: 2391-2405, 2016).

In other embodiments, the patients to be treated according to the methods of the invention have or are diagnosed with chronic myeloid leukemia (also known as chronic myelogenous leukemia, chronic granulocytic leukemia and chronic myelocytic leukemia). Patients who have chronic myeloid leukemia may not initially have any of the leukemia symptoms described above, but rather are suspected of having this disease based on abnormally elevated levels of white blood cells in the blood. Diagnosis of chronic myeloid leukemia is made in patients having: increased white blood cell count, decreased red blood cell count, and increase or decrease in platelet levels in blood samples; presence of leukemic blast cells in blood or bone marrow; and/or presence of cells in bone marrow having chromosomal abnormalities, such as the Philadelphia chromosome (BCR-ABL1 fusion). Cytogenetic analysis, fluorescence in situ hybridization (FISH), or polymerase chain reaction (PCR) are all methods that can be used to assess chromosomal abnormalities in cells obtained from blood or bone marrow samples from patients suspected of having chronic myeloid leukemia. The diagnosis of chronic myeloid leukemia is preferably made by a hematopathologist with experience in diagnosing leukemias by, preferably applying the WHO classification of myeloid neoplasms and acute leukemia (see Table 1 of Arber et al., Blood, Vol. 127: 2391-2405, 2016).

In certain embodiments, the patients to be treated according to the methods of the invention have or are diagnosed with relapsed and/or refractory myeloid leukemia, particularly relapsed and/or refractory acute myeloid leukemia. As used herein, the term “relapsed myeloid leukemia” refers to the return of signs and symptoms of myeloid leukemia, particularly acute myeloid leukemia, such as the return of leukemic blast cells in bone marrow and decline of normal blood cells, after a patient has experienced a remission from the disease. For instance, following initial treatment with an anti-leukemic therapy, such as a standard chemotherapy regimen (e.g. cytarabine and/or anthracyclines), and/or a hematopoietic stem cell transplant, a myeloid leukemia patient may go into remission with no signs or symptoms of leukemic disease, but then suffer a relapse with return of leukemic cells and once again require treatment for myeloid leukemia. A patient may be considered to have relapsed myeloid leukemia if one or more of the following criteria are met following a remission period: (i) recurrence of leukemic blast cells in the bone marrow of 5% or greater, (ii) recurrence of leukemic blast cells in the peripheral blood, (iii) recurrence of leukemia at an extramedullary site (i.e. outside the bone marrow), (iv) recurrence of pre-treatment characteristic signs of morphological dysplasia, or (v) recurrence of Auer rods (large, crystalline cytoplasmic inclusion bodies) in bone marrow cells or blood cells. In certain embodiments, the myeloid leukemia patient relapses following a duration of first remission less than a year, for example, a duration of first remission of about 3 months to about 11 months. In other embodiments, the myeloid leukemia patient relapses following a duration of first remission greater than a year.

As used herein, the term “refractory myeloid leukemia” refers to myeloid leukemia, particularly acute myeloid leukemia, that does not respond or only partially responds to an anti-leukemic therapy for myeloid leukemia, such as chemotherapy and/or a hematopoietic stem cell transplant. Thus, a patient in whom the presence of leukemic disease (e.g. leukemic blast cells in bone marrow or peripheral blood) is detected following a course of therapy for myeloid leukemia is considered to have refractory myeloid leukemia.

The methods described herein are also applicable to other types of myeloid malignancies, which are clonal diseases arising in hematopoietic stem or progenitor cells. Other types of myeloid malignancies that may be treated according to the methods of the invention include, but are not limited to, myelodysplastic syndrome, polycythemia vera, essential thrombocythemia, primary myelofibrosis, acute basophilic leukemia, acute eosinophilic leukemia, chronic eosinophilic leukemia, chronic neutrophilic leukemia, acute megakaryoblastic leukemia, acute erythroid leukemia, hypereosinophilic syndrome, mast cell disease, acute panmyeloic leukemia, and myeloid sarcoma. Accordingly, the present invention also provides methods for treating any of the aforementioned myeloid malignancies by administering an anti-CD33×anti-CD3 bispecific antibody construct to a patient in need thereof with any of the dosage regimens described herein.

Administration of the anti-CD33×anti-CD3 bispecific antibody construct according to the methods of the invention is for the treatment of myeloid leukemia or other myeloid malignancies. The term “treatment” or “treat” as used herein refers to the application or administration of the bispecific antibody construct to a patient who has or is diagnosed with myeloid leukemia or myeloid malignancy, has a symptom of myeloid leukemia or myeloid malignancy, is at risk of developing myeloid leukemia or myeloid malignancy, or has a predisposition to myeloid leukemia or myeloid malignancy for the purpose of curing, healing, alleviating, relieving, altering, ameliorating, or improving myeloid leukemia or myeloid malignancy, one or more symptoms of myeloid leukemia or myeloid malignancy, the risk of developing myeloid leukemia or myeloid malignancy, or predisposition toward myeloid leukemia or myeloid malignancy. The term “treatment” encompasses any improvement of the disease in the patient, including the slowing or stopping of the progression of myeloid leukemia or myeloid malignancy in the patient, a decrease in the number or severity of the symptoms of myeloid leukemia or myeloid malignancy, or an increase in frequency or duration of periods where the patient is free from the symptoms of myeloid leukemia or myeloid malignancy. The term “patient” includes human patients.

In certain embodiments of the methods of the invention, administration of the anti-CD33×anti-CD3 bispecific antibody construct reduces the percentage of leukemic blast cells (myeloblasts) in the bone marrow of the patient by at least about 30%, at least about 40%, at least about 50%, at least about 60%, at least about 70%, at least about 75%, at least about 80%, at least about 85%, at least about 90%, or at least about 95%, or about 100% relative to the percentage of leukemic blast cells in the bone marrow prior to the start of the treatment (i.e. prior to the administration of the anti-CD33×anti-CD3 bispecific antibody construct). In some embodiments, administration of the anti-CD33×anti-CD3 bispecific antibody construct reduces the percentage of leukemic blast cells (myeloblasts) in the bone marrow of the patient by 50% or greater relative to the percentage of leukemic blast cells in the bone marrow prior to the start of the treatment. In other embodiments, administration of the anti-CD33×anti-CD3 bispecific antibody construct reduces the percentage of leukemic blast cells (myeloblasts) in the bone marrow of the patient by 80% or greater relative to the percentage of leukemic blast cells in the bone marrow prior to the start of the treatment. Methods of assessing the percentage of leukemic blast cells in bone marrow samples from a patient are known to those of skill in the art and can involve similar methods used in the diagnosis of myeloid leukemia.

In some embodiments of the methods of the invention, administration of the anti-CD33×anti-CD3 bispecific antibody construct induces a complete remission of disease in at least about 20%, at least about 30%, at least about 40%, at least about 50%, at least about 60%, at least about 70%, at least about 75%, at least about 80%, at least about 85%, at least about 90%, or at least about 95% of myeloid leukemia patients. Complete remission (CR) in the context of the invention refers to the condition characterized by all of the following: (i) less than 5% leukemic blast cells in the bone marrow, (ii) absence of leukemic blast cells with Auer rods, and (iii) absence of extramedullary leukemic disease. Complete remission can include complete remission with hematologic recovery, in which case the patient exhibits absolute neutrophil counts≥1,000 cells per μL and platelet counts≥100,000 cells per μL and does not require red cell transfusions in addition to the criteria specified above. In some embodiments, administration of the anti-CD33×anti-CD3 bispecific antibody construct induces complete remission with incomplete or partial hematologic recovery in a patient. Complete remission with incomplete hematologic recovery (CRi) refers to complete remission as characterized by the three criteria above, but the patient has residual neutropenia (e.g. an absolute neutrophil count<1,000 cells per μL) and/or residual thrombocytopenia (e.g. platelet count<100,000 cells per μL). Complete remission with partial hematologic recovery (CRh) refers to complete remission as characterized by the three criteria above, but the patient exhibits absolute neutrophil counts>500 cells per μL and platelet counts>50,000 cells per μL.

Efficacy of the therapeutic regimens described herein can also be assessed in terms of time to response to treatment, duration of response to treatment, or time to progression of leukemic disease. For instance, in some embodiments, administration of the anti-CD33×anti-CD3 bispecific antibody construct reduces time to response as compared to the time to response observed for a standard chemotherapy regimen (e.g. cytarabine and/or anthracyclines). As used in this context, “time to response” refers to the period of time between initiation of treatment and CR with hematologic recovery, CRi, CRh, or reduction in leukemic blast cells in bone marrow by at least 50%. In other embodiments, administration of the anti-CD33×anti-CD3 bispecific antibody construct increases the duration of response to treatment as compared to the duration of response to treatment observed for a standard chemotherapy regimen (e.g. cytarabine and/or anthracyclines). “Duration of response” as the term is used herein refers to the period of time from remission (CR, CR with hematologic recovery, CRi, or CRh) to relapse of leukemic disease as described herein. Thus, in some embodiments, administration of the anti-CD33×anti-CD3 bispecific antibody construct according to the methods of the invention prevents or delays relapse of leukemic disease in the patient.

In certain embodiments, administration of the anti-CD33×anti-CD3 bispecific antibody construct increases the time to progression of leukemic disease in a patient as compared to the time to progression of leukemic disease observed for a standard chemotherapy regimen (e.g. cytarabine and/or anthracyclines). Leukemic disease is considered to progress when any of the following occur: (i) greater than 50% increase in leukemic blast cells in bone marrow from prior assessment and at least 20% leukemic blast cells in bone marrow, (ii) greater than 50% increase in leukemic blast cells in peripheral blood from prior assessment and at least an absolute leukemic blast cell count in peripheral blood of 1000 cells per μL, or (iii) development of extramedullary leukemic disease or additional sites of extramedullary leukemic disease. “Time to progression of leukemic disease” refers to the period of time between initiation of treatment and the time at which leukemic disease is considered to progress as described by the criteria above. In some embodiments, administration of the anti-CD33×anti-CD3 bispecific antibody construct according to the methods of the invention prevents or delays progression of leukemic disease in the patient.

In some aspects, the methods of the invention comprise administering to a patient a pharmaceutical composition comprising a therapeutically effective amount of an anti-CD33×anti-CD3 bispecific antibody construct. A “therapeutically effective amount” refers to an amount sufficient to treat or ameliorate myeloid leukemia or one or more of its symptoms, particularly a state or symptoms associated with myeloid leukemia, or otherwise prevent, hinder, retard or reverse the progression of myeloid leukemia or any other undesirable symptom associated with myeloid leukemia in any way whatsoever. Suitable dosages of the anti-CD33×anti-CD3 bispecific antibody construct for each of the initiation and maintenance cycles are described in more detail herein. In certain embodiments, a therapeutically effective amount of an anti-CD33×anti-CD3 bispecific antibody construct is an amount sufficient to induce complete remission of myeloid leukemia in the patient. In these and other embodiments, a therapeutically effective amount of an anti-CD33×anti-CD3 bispecific antibody construct is an amount sufficient to prevent or delay relapse of myeloid leukemia in the patient. In still other embodiments, a therapeutically effective amount of an anti-CD33×anti-CD3 bispecific antibody construct is an amount sufficient to prevent or delay the progression of myeloid leukemia in the patient.

Generally, the methods of the invention comprise administering an anti-CD33×anti-CD3 bispecific antibody construct to the patient in one or more treatment cycles. A “treatment cycle” or “cycle” refers to a period of administration of the bispecific antibody construct at specific dosages and dosing intervals. According to the methods of the invention, a patient can receive multiple treatment cycles (e.g. 2, 3, 4, 5, 6, 7, 8, 9, 10, 11, 12, 13, 14 or more cycles). The treatment cycles can be administered to the patient consecutively with no break or period without administration of the bispecific antibody construct between the cycles. Alternatively, a period without administration of the bispecific antibody construct (e.g. a “treatment-free period” or “break”) can be employed between the treatment cycles. The length of the treatment-free period can be adjusted based on the patient's characteristics and/or response to treatment. For instance, a treatment-free period may be employed for the patient to recover from low or suppressed hematological cell counts, for example, if the patient is exhibiting neutropenia, thrombocytopenia, or anemia.

In a preferred embodiment, the methods of the invention comprise administering an anti-CD33×anti-CD3 bispecific antibody construct to the patient in at least one initiation cycle and at least one maintenance cycle. As used herein, an “initiation cycle” is a treatment cycle in which the bispecific antibody construct is administered at a dosing frequency designed to rapidly increase exposure of the patient to a therapeutically effective dose of the anti-CD33×anti-CD3 bispecific antibody construct. An initiation cycle is preferably administered to a patient as the first treatment cycle when the patient begins a course of treatment with the anti-CD33×anti-CD3 bispecific antibody construct. An initiation cycle may also be administered to a patient when the patient re-starts a course of treatment with the anti-CD33×anti-CD3 bispecific antibody construct, for example, following a treatment-free period or a relapse of leukemic disease. Although administration of one initiation cycle will typically be sufficient, in some embodiments of the methods of the invention, administration of two or more initiation cycles is contemplated.

In certain embodiments of the methods of the invention, the initiation cycle comprises administering an anti-CD33×anti-CD3 bispecific antibody construct at one or more doses of about 15 μg to about 1000 μg at each dosing interval. For instance, the initiation cycle comprises administering an anti-CD33×anti-CD3 bispecific antibody construct at one or more doses of about 18 μg to about 480 μg, about 36 μg to about 480 μg, about 72 μg to about 200 μg, about 100 μg to about 180 μg, about 110 μg to about 240 μg, about 110 μg to about 360 μg, about 72 μg to about 480 μg, about 18 μg to about 240 μg, about 36 μg to about 240 μg, about 150 μg to about 360 μg, about 180 μg to about 480 μg, about 150 μg to about 480 μg, about 100 μg to about 800 μg, or about 200 μg to about 600 μg at each dosing interval. In one embodiment, the initiation cycle comprises administering an anti-CD33×anti-CD3 bispecific antibody construct at one or more doses of about 18 μg to about 480 μg at each dosing interval. In another embodiment, the initiation cycle comprises administering an anti-CD33×anti-CD3 bispecific antibody construct at one or more doses of about 36 μg to about 480 μg at each dosing interval. In another embodiment, the initiation cycle comprises administering an anti-CD33×anti-CD3 bispecific antibody construct at one or more doses of about 110 μg to about 360 μg at each dosing interval. In yet another embodiment, the initiation cycle comprises administering an anti-CD33×anti-CD3 bispecific antibody construct at one or more doses of about 100 μg to about 180 μg at each dosing interval. In still another embodiment, the initiation cycle comprises administering an anti-CD33×anti-CD3 bispecific antibody construct at one or more doses of about 72 μg to about 480 μg at each dosing interval. In another embodiment, the initiation cycle comprises administering an anti-CD33×anti-CD3 bispecific antibody construct at one or more doses of about 18 μg to about 240 μg at each dosing interval.

In some embodiments, the dose of the bispecific antibody construct administered during the initiation cycle may be the same at each dosing interval (e.g. a fixed dose for the entire cycle). Accordingly, the specific dose of an anti-CD33×anti-CD3 bispecific antibody construct is administered initially and at all subsequent administrations at the prescribed dosing interval for the duration of the initiation cycle. By way of example, if the dosing interval was daily, administration of the anti-CD33×anti-CD3 bispecific antibody construct according to this embodiment would entail administration of the specific dose (e.g. 110 μg) once per day for the duration of the initiation cycle.

In alternative embodiments, the dose of the bispecific antibody construct administered during the initiation cycle may change from one dosing interval to the next. For instance, in some embodiments, the dose of the bispecific antibody construct administered during the initiation cycle increases at least once at one or more intervals during the cycle (i.e. step dosing). Step dosing refers to the administration of increasing doses of a drug during a treatment cycle, for example, to control exposure of the patient to the drug to avoid or limit adverse effects. The step dosing regimen employed during an initiation cycle may comprise one or more dosage steps (e.g. one or more dose increases). For instance, in one embodiment, the initiation cycle comprises administering the bispecific antibody construct at a first dose for one or more intervals followed by administration of the bispecific antibody construct at a second dose for one or more intervals, wherein the second dose is greater than the first dose. In a related embodiment, a second dosage step may be employed such that the initiation cycle further comprises administering the bispecific antibody construct at a third dose for one or more intervals following administration of the second dose, wherein the third dose is greater than the second dose. In another embodiment, a third dosage step is employed such that the initiation cycle further comprises administering the bispecific antibody construct at a fourth dose for one or more intervals following administration of the third dose, wherein the fourth dose is greater than the third dose. In yet another embodiment, a fourth dosage step is employed such that the initiation cycle further comprises administering the bispecific antibody construct at a fifth dose for one or more intervals following administration of the fourth dose, wherein the fifth dose is greater than the fourth dose. In still another embodiment, a fifth dosage step is employed such that the initiation cycle further comprises administering the bispecific antibody construct at a sixth dose for one or more intervals following administration of the fifth dose, wherein the sixth dose is greater than the fifth dose. One or more dosage steps can be used, for example, 2, 3, 4, 5, 6, 7, 8, 9, or 10 or more dosage steps. In some embodiments, the step dosing regimen employed during the initiation cycle may comprise up to six dosage steps (i.e. seven different doses administered), e.g. 1, 2, 3, 4, 5, or 6 dosage steps. In other embodiments, the step dosing regimen employed during the initiation cycle may comprise up to seven dosage steps (i.e. eight different doses administered), e.g. 1, 2, 3, 4, 5, 6, or 7 dosage steps.

The step doses of an anti-CD33×anti-CD3 bispecific antibody construct can be any of the doses within the ranges specified above for administration during the initiation cycle. For instance, the step doses may range from about 15 μg to about 1000 μg, such as from about 18 μg to about 480 μg, from about 36 μg to about 480 μg, from about 110 μg to about 360 μg, from about 100 μg to about 180 μg, from about 72 μg to about 480 μg, from about 18 μg to about 240 fig, from about 36 μg to about 240 μg, or from about 150 μg to about 480 μg. In some embodiments, the step doses may increase proportionally over the range. In alternative embodiments, the step doses may increase in smaller or larger steps over the range, e.g. small steps at the earlier step doses and larger steps at the later step doses. In certain embodiments in which a step dosing regimen is employed during the initiation cycle, the first dose of an anti-CD33×anti-CD3 bispecific antibody construct administered to the patient is from about 18 μg to about 150 μg and the second dose is from about 110 μg to about 240 μg, wherein the second dose is greater than the first dose. In other embodiments in which a step dosing regimen is employed during the initiation cycle, the first dose of an anti-CD33×anti-CD3 bispecific antibody construct administered to the patient is from about 36 μg to about 150 μg and the second dose is from about 110 μg to about 240 μg, wherein the second dose is greater than the first dose. In still other embodiments in which a step dosing regimen is employed during the initiation cycle, the first dose of an anti-CD33×anti-CD3 bispecific antibody construct administered to the patient is from about 18 μg to about 110 μg and the second dose is from about 72 μg to about 160 μg, wherein the second dose is greater than the first dose. In one such embodiment, the first dose is about 36 μg and the second dose is about 72 μg.

In embodiments in which the step dosing regimen comprises two dosage steps (i.e. three different doses administered), the first dose of an anti-CD33×anti-CD3 bispecific antibody construct is from about 18 μg to about 150 μg, the second dose is from about 110 μg to about 240 μg, and the third dose is from about 150 μg to about 360 μg, wherein the third dose is greater than the second dose and the second dose is greater than the first dose. In other embodiments in which the step dosing regimen comprises two dosage steps (i.e. three different doses administered), the first dose of an anti-CD33×anti-CD3 bispecific antibody construct is from about 36 μg to about 150 μg, the second dose is from about 110 μg to about 240 μg, and the third dose is from about 150 μg to about 360 μg, wherein the third dose is greater than the second dose and the second dose is greater than the first dose. In certain embodiments in which the step dosing regimen comprises three dosage steps (i.e. four different doses administered), the first dose of an anti-CD33×anti-CD3 bispecific antibody construct is from about 18 μg to about 150 μg, the second dose is from about 110 μg to about 240 μg, the third dose is from about 150 μg to about 360 μg, and the fourth dose is from about 180 μg to about 480 μg, wherein the fourth dose is greater than the third dose, the third dose is greater than the second dose, and the second dose is greater than the first dose. In certain other embodiments in which the step dosing regimen comprises three dosage steps (i.e. four different doses administered), the first dose of an anti-CD33×anti-CD3 bispecific antibody construct is from about 36 μg to about 150 μg, the second dose is from about 110 μg to about 240 μg, the third dose is from about 150 μg to about 360 μg, and the fourth dose is from about 180 μg to about 480 μg, wherein the fourth dose is greater than the third dose, the third dose is greater than the second dose, and the second dose is greater than the first dose. In yet other embodiments in which the step dosing regimen comprises three dosage steps (i.e. four different doses administered), the first dose of an anti-CD33×anti-CD3 bispecific antibody construct is from about 18 μg to about 110 μg, the second dose is from about 36 μg to about 160 μg, the third dose is from about 72 μg to about 240 μg, and the fourth dose is from about 110 μg to about 480 μg, wherein the fourth dose is greater than the third dose, the third dose is greater than the second dose, and the second dose is greater than the first dose. In one particular embodiment, the first dose is about 18 μg, the second dose is about 36 μg, the third dose is about 72 μg, and the fourth dose is about 110 μg.

In some embodiments in which the step dosing regimen comprises four dosage steps (i.e. five different doses administered), the first dose can be from about 18 μg to about 110 μg, the second dose can be from about 36 μg to about 160 μg, the third dose can be from about 72 μg to about 240 μg, the fourth dose can be from about 110 μg to about 360 μg, and the fifth dose can be from about 160 μg to about 480 μg, wherein the fifth dose is greater than the fourth dose, the fourth dose is greater than the third dose, the third dose is greater than the second dose, and the second dose is greater than the first dose. In one such embodiment, the first dose is about 18 μg, the second dose is about 36 μg, the third dose is about 72 μg, the fourth dose is about 110 μg, and the fifth dose is about 160 μg. In embodiments in which the step dosing regimen comprises five dosage steps (i.e. six different doses administered), the first dose can be from about 18 μg to about 72 μg, the second dose can be from about 36 μg to about 110 μg, the third dose can be from about 72 μg to about 160 μg, the fourth dose can be from about 110 μg to about 240 μg, the fifth dose can be from about 160 μg to about 360 μg, and the sixth dose can be from about 240 μg to about 480 μg, wherein the sixth dose is greater than the fifth dose, the fifth dose is greater than the fourth dose, the fourth dose is greater than the third dose, the third dose is greater than the second dose, and the second dose is greater than the first dose. In certain such embodiments, the first dose is about 18 μg, the second dose is about 36 μg, the third dose is about 72 μg, the fourth dose is about 110 μg, the fifth dose is about 160 μg, and the sixth dose is about 240 μg.

Any of the doses of an anti-CD33×anti-CD3 bispecific antibody construct described herein for administration during the initiation cycle can be administered at an interval of 1 day to 4 days. For instance, in one embodiment, the initiation cycle comprises administering the dose of an anti-CD33×anti-CD3 bispecific antibody construct once per day (e.g. daily, QD dosing). In another embodiment, the initiation cycle comprises administering the dose of an anti-CD33×anti-CD3 bispecific antibody construct once every other day (e.g. Q2D dosing). In yet another embodiment, the initiation cycle comprises administering the dose of an anti-CD33×anti-CD3 bispecific antibody construct once every three days (e.g. Q3D dosing). In still another embodiment, the initiation cycle comprises administering the dose of an anti-CD33×anti-CD3 bispecific antibody construct once every four days (e.g. Q4D dosing). In some embodiments in which a step dosing regimen is employed during the initiation cycle, the dose can be increased at each dosing interval. For example, for a dosing interval of once per day (e.g. daily), the step dosing regimen may comprise administering a first dose at day 1 (D1), a second higher dose at day 2 (D2), a third higher dose at day 3 (D3), and so on. In other embodiments in which a step dosing regimen is employed during the initiation cycle, the dose may be held constant for two or more dosing intervals and subsequently increased at later dosing intervals. In one such embodiment, for a dosing interval of once per day (e.g. daily), the step dosing regimen may comprise administering a first dose at D1 and D2, a second higher dose at D3 and day 4 (D4), a third higher dose at day 5 (D5) and day 6 (D6), and so on. In another such embodiment, for a dosing interval of once per day (e.g. daily), the step dosing regimen may comprise administering a first dose at D1, a second higher dose at D2 and D3, a third higher dose at D4 and D5, and so on. When a step dosing regimen is employed during the initiation cycle, a daily dosing interval (e.g. once per day) is preferred in some embodiments.

In certain embodiments of the methods of the invention, the duration of the initiation cycle (e.g. first period of time) is from about 5 days to about 30 days, for example, from about 7 days to about 28 days, from about 7 days to about 14 days, from about 14 days to about 28 days, or from about 5 days to about 15 days. Thus, in some embodiments, the initiation cycle comprises administering an anti-CD33×anti-CD3 bispecific antibody construct at one or more of the doses described herein at an interval of 1 day to 4 days for a first period of time, wherein the first period of time is about 7 days to about 14 days. In other embodiments, the initiation cycle comprises administering an anti-CD33×anti-CD3 bispecific antibody construct at one or more of the doses described herein at an interval of 1 day to 4 days for a first period of time, wherein the first period of time is about 14 days to about 28 days. In one embodiment of the methods of the invention, the initiation cycle comprises administering the bispecific antibody construct at one or more of the doses described herein once per day (e.g. daily) for 7 days. In another embodiment, the initiation cycle comprises administering the bispecific antibody construct at one or more of the doses described herein once per day (e.g. daily) for 14 days. In yet another embodiment, the initiation cycle comprises administering the bispecific antibody construct at one or more of the doses described herein once every other day (e.g. Q2D) for 14 days. In still another embodiment, the initiation cycle comprises administering the bispecific antibody construct at one or more of the doses described herein once every three days (e.g. Q3D) for 14 days. In another embodiment, the initiation cycle comprises administering the bispecific antibody construct at one or more of the doses described herein once every four days (e.g. Q4D) for 7 days (e.g. antibody construct administered twice within a week at D1 and D5).

In certain embodiments, the methods of the invention comprise administering at least one maintenance cycle of an anti-CD33×anti-CD3 bispecific antibody construct to the patient after administration of one or more initiation cycles. As used herein, a “maintenance cycle” is a treatment cycle in which the bispecific antibody construct is administered at a dosing frequency designed to maintain a threshold level of exposure of the anti-CD33×anti-CD3 bispecific antibody construct at therapeutic levels in the patient. In a preferred embodiment, the dosing frequency employed in the maintenance cycle is lower than the dosing frequency employed in the initiation cycle (i.e. the dosing interval in the maintenance cycle is longer than the dosing interval in the initiation cycle). In certain embodiments, the maintenance cycle is administered immediately after the completion of one or more initiation cycles to sustain exposure of the patient to the anti-CD33×anti-CD3 bispecific antibody construct. Accordingly, in such embodiments, there are no treatment-free periods or breaks between the end of the initiation cycle and the start of the maintenance cycle. In one such embodiment, the maintenance cycle is initiated the following day after completing the initiation cycle.

Multiple maintenance cycles (e.g. 2, 3, 4, 5, 6, 7, 8, 9, 10, 11, 12 or more cycles) can be administered to the patient depending on the desired duration of treatment for that patient. For instance, the patient may receive maintenance cycles of the anti-CD33×anti-CD3 bispecific antibody construct until the patient achieves a desired level of response, such as complete remission of leukemic disease. In some embodiments, two or more maintenance cycles are administered to the patient. In other embodiments, four or more maintenance cycles are administered to the patient. In still other embodiments, six to twelve maintenance cycles are administered to the patient. In preferred embodiments, the maintenance cycles are administered consecutively with no treatment-free periods between the maintenance cycles. If a treatment interruption is necessary, ideally the duration of the treatment-free period will be no greater than twice the dosing interval employed in the maintenance cycle. By way of example, if the dosing interval employed in the maintenance cycle is once per week (e.g. weekly), the treatment-free period will preferably be about 14 days or less.

In certain embodiments of the methods of the invention, the maintenance cycle comprises administering an anti-CD33×anti-CD3 bispecific antibody construct at a dose of about 30 μg to about 1000 μg once or twice every 7 days (e.g. once per week or twice per week). For instance, the maintenance cycle comprises administering an anti-CD33×anti-CD3 bispecific antibody construct at a dose of about 36 μg to about 480 μg, about 72 μg to about 200 μg, about 72 μg to about 360 μg, about 100 μg to about 180 μg, about 110 μg to about 240 μg, about 110 μg to about 360 μg, about 72 μg to about 480 μg, about 36 μg to about 240 μg, about 150 μg to about 360 μg, about 180 μg to about 480 μg, about 150 μg to about 480 μg, about 100 μg to about 800 fig, or about 200 μg to about 600 μg once or twice every 7 days. In one embodiment, the maintenance cycle comprises administering an anti-CD33×anti-CD3 bispecific antibody construct at a dose of about 36 μg to about 480 μg once or twice every 7 days. In another embodiment, the maintenance cycle comprises administering an anti-CD33×anti-CD3 bispecific antibody construct at a dose of about 110 μg to about 240 μg once or twice every 7 days. In yet another embodiment, the maintenance cycle comprises administering an anti-CD33×anti-CD3 bispecific antibody construct at a dose of about 72 μg to about 360 μg once or twice every 7 days. In certain embodiments, the dose of the bispecific antibody construct administered during the maintenance cycle is the same as the highest dose of the bispecific antibody construct administered during the initiation cycle. In some embodiments in which a step dosing regimen is employed during the initiation cycle, the dose administered during the maintenance cycle is the same as the last step dose administered during the initiation cycle. In some embodiments, the dose of the bispecific antibody construct administered during the maintenance cycle is the same at each weekly or twice weekly dosing interval (e.g. a fixed dose for the entire maintenance cycle). In these and other embodiments, the dose and dosing frequency of the bispecific antibody construct administered during the maintenance cycle is the same from one maintenance cycle to the next maintenance cycle. In one particular embodiment of the methods of the invention, the maintenance cycle comprises administering the dose of an anti-CD33×anti-CD3 bispecific antibody construct once per week (e.g. once every 7 days, weekly, or QW dosing). In another particular embodiment, the maintenance cycle comprises administering the dose of an anti-CD33×anti-CD3 bispecific antibody construct twice per week (e.g. twice every 7 days).

According to some embodiments of the methods of the invention, the duration of the maintenance cycle (e.g. second period of time) is from about 14 days to about 60 days, for example, from about 14 days to about 28 days, from about 28 days to about 56 days, from about 14 days to about 21 days, from about 15 days to about 30 days, or from about 30 days to about 60 days. Thus, in some embodiments, the maintenance cycle comprises administering an anti-CD33×anti-CD3 bispecific antibody construct at a dose described herein once or twice every 7 days for a second period of time, wherein the second period of time is about 14 days to about 28 days. In one embodiment of the methods of the invention, the maintenance cycle comprises administering the bispecific antibody construct at a dose described herein once every 7 days (e.g. weekly, QW) for 14 days. In another embodiment, the maintenance cycle comprises administering the bispecific antibody construct at a dose described herein once every 7 days (e.g. weekly, QW) for 28 days. In yet another embodiment, the maintenance cycle comprises administering the bispecific antibody construct at a dose described herein twice every 7 days (e.g. twice per week) for 14 days. In still another embodiment, the maintenance cycle comprises administering the bispecific antibody construct at a dose described herein twice every 7 days (e.g. twice per week) for 28 days.

In some embodiments, the methods of the invention comprise administering to the patient at least one initiation cycle and at least one maintenance cycle of an anti-CD33×anti-CD3 bispecific antibody construct, wherein the initiation cycle comprises administering the bispecific antibody construct at a dose of about 110 μg to about 360 μg once every four days for 7 days, and wherein the maintenance cycle comprises administering the bispecific antibody construct at a dose of about 110 μg to about 360 μg once every 7 days for 14 days, and wherein the maintenance cycle is administered 7-9 days after the initiation cycle. An exemplary dosing schedule according to these embodiments comprises administration of the dose (e.g. 110 μg) of the bispecific antibody construct at day 1 (D1) and day 5 (D5) of a 7-day initiation cycle, followed by a treatment-free period of 7 days, followed by administration of the dose (e.g. 110 fig) of the bispecific antibody construct at day 1 (D1) and day 8 (D8) of a 14-day maintenance cycle. Thus, according to this dosing regimen, for the 28-day period starting with the first dose of the initiation cycle, the patient would be administered the bispecific antibody construct on each of D1, D5, day 15 (D15), and day 22 (D22).

In certain embodiments, the methods of the invention comprise administering to the patient at least one initiation cycle and at least one maintenance cycle of an anti-CD33×anti-CD3 bispecific antibody construct, wherein the initiation cycle comprises administering the bispecific antibody construct at a dose of about 18 μg to about 480 μg once per day (e.g. daily) for 7 days or 14 days, and wherein the maintenance cycle comprises administering the bispecific antibody construct at a dose of about 72 μg to about 480 μg once every 7 days (e.g. weekly) for 14 days, and wherein the maintenance cycle is administered the following day after completing the initiation cycle. An exemplary dosing schedule according to these embodiments comprises administration of a dose of the bispecific antibody construct once per day for each day from D1 to day 7 (D7) of a 7-day initiation cycle, followed by administration of a dose of the bispecific antibody construct at D1 and D8 of a 14-day maintenance cycle. Thus, according to this dosing regimen, for the 21-day period including both the 7-day initiation cycle and the 14-day maintenance cycle, the patient would be administered the bispecific antibody construct on each of D1 to D7, D8, and D15. Another exemplary dosing schedule according to these embodiments comprises administration of a dose of the bispecific antibody construct once per day for each day from D1 to day 14 (D14) of a 14-day initiation cycle, followed by administration of a dose of the bispecific antibody construct at D1 and D8 of a 14-day maintenance cycle. According to this dosing regimen, for the 28-day period including both the 14-day initiation cycle and the 14-day maintenance cycle, the patient would be administered the bispecific antibody construct on each of D1 to D14, D15, and D22.

In other embodiments, the methods of the invention comprise administering to the patient at least one initiation cycle and at least one maintenance cycle of an anti-CD33×anti-CD3 bispecific antibody construct, wherein the initiation cycle comprises administering the bispecific antibody construct at a dose of about 18 μg to about 480 μg once per day (e.g. daily) for 7 days or 14 days, and wherein the maintenance cycle comprises administering the bispecific antibody construct at a dose of about 72 μg to about 480 μg twice every 7 days (e.g. twice per week) for 14 days, and wherein the maintenance cycle is administered the following day after completing the initiation cycle. An exemplary dosing schedule according to these embodiments comprises administration of a dose of the bispecific antibody construct once per day for each day from D1 to D7 of a 7-day initiation cycle, followed by administration of a dose of the bispecific antibody construct at D1, D4, D8, and day 11 (D11) of a 14-day maintenance cycle. Therefore, according to this dosing regimen, for the 21-day period including both the 7-day initiation cycle and the 14-day maintenance cycle, the patient would be administered the bispecific antibody construct on each of D1 to D7, D8, D11, D15, and day 18 (D18). Another exemplary dosing schedule according to these embodiments comprises administration of a dose of the bispecific antibody construct once per day for each day from D1 to D14 of a 14-day initiation cycle, followed by administration of a dose of the bispecific antibody construct at D1, D4, D8, and D1l of a 14-day maintenance cycle. According to this dosing regimen, for the 28-day period including both the 14-day initiation cycle and the 14-day maintenance cycle, the patient would be administered the bispecific antibody construct on each of D1 to D14, D15, D18, D22, and D25.

In alternative embodiments, the methods of the invention comprise administering to the patient at least one initiation cycle and at least one maintenance cycle of an anti-CD33×anti-CD3 bispecific antibody construct, wherein the initiation cycle comprises administering the bispecific antibody construct at a dose of about 72 μg to about 480 μg once per day (e.g. daily) for 7 days or 14 days, and wherein the maintenance cycle comprises administering the bispecific antibody construct at a dose of about 72 μg to about 480 μg once every 7 days (e.g. weekly) for 14 days, and wherein the maintenance cycle is administered the following day after completing the initiation cycle. An exemplary dosing schedule according to these embodiments comprises administration of the dose (e.g. 72 μg) of the bispecific antibody construct once per day for each day from D1 to day 7 (D7) of a 7-day initiation cycle, followed by administration of the dose (e.g. 72 μg) of the bispecific antibody construct at D1 and D8 of a 14-day maintenance cycle. Thus, according to this dosing regimen, for the 21-day period including both the 7-day initiation cycle and the 14-day maintenance cycle, the patient would be administered the bispecific antibody construct on each of D1 to D7, D8, and D15. Another exemplary dosing schedule according to these embodiments comprises administration of the dose (e.g. 72 μg) of the bispecific antibody construct once per day for each day from D1 to day 14 (D14) of a 14-day initiation cycle, followed by administration of the dose (e.g. 72 μg) of the bispecific antibody construct at D1 and D8 of a 14-day maintenance cycle. According to this dosing regimen, for the 28-day period including both the 14-day initiation cycle and the 14-day maintenance cycle, the patient would be administered the bispecific antibody construct on each of D1 to D14, D15, and D22.

In certain other embodiments, the methods of the invention comprise administering to the patient at least one initiation cycle and at least one maintenance cycle of an anti-CD33×anti-CD3 bispecific antibody construct, wherein the initiation cycle comprises administering the bispecific antibody construct at a dose of about 72 μg to about 480 μg once per day (e.g. daily) for 7 days or 14 days, and wherein the maintenance cycle comprises administering the bispecific antibody construct at a dose of about 72 μg to about 480 μg twice every 7 days (e.g. twice per week) for 14 days, and wherein the maintenance cycle is administered the following day after completing the initiation cycle. An exemplary dosing schedule according to these embodiments comprises administration of the dose (e.g. 72 μg) of the bispecific antibody construct once per day for each day from D1 to D7 of a 7-day initiation cycle, followed by administration of the dose (e.g. 72 μg) of the bispecific antibody construct at D1, D4, D8, and day 11 (D11) of a 14-day maintenance cycle. Therefore, according to this dosing regimen, for the 21-day period including both the 7-day initiation cycle and the 14-day maintenance cycle, the patient would be administered the bispecific antibody construct on each of D1 to D7, D8, D11, D15, and day 18 (D18). Another exemplary dosing schedule according to these embodiments comprises administration of the dose (e.g. 72 μg) of the bispecific antibody construct once per day for each day from D1 to D14 of a 14-day initiation cycle, followed by administration of the dose (e.g. 72 fig) of the bispecific antibody construct at D1, D4, D8, and D11 of a 14-day maintenance cycle. According to this dosing regimen, for the 28-day period including both the 14-day initiation cycle and the 14-day maintenance cycle, the patient would be administered the bispecific antibody construct on each of D1 to D14, D15, D18, D22, and D25.

In one embodiment, the methods of the invention comprise administering to the patient at least one initiation cycle and at least one maintenance cycle of an anti-CD33×anti-CD3 bispecific antibody construct, wherein the initiation cycle comprises administering the bispecific antibody construct at a first dose of 36 μg once per day (e.g. daily) for 2 days, followed by administration of the bispecific antibody construct at a second dose of 72 μg once per day for 12 days, and wherein the maintenance cycle comprises administering the bispecific antibody construct at a dose of 72 μg once or twice every 7 days (e.g. once or twice per week) for 14 days, and wherein the maintenance cycle is administered the following day after completing the initiation cycle.

In another embodiment, the methods of the invention comprise administering to the patient at least one initiation cycle and at least one maintenance cycle of an anti-CD33×anti-CD3 bispecific antibody construct, wherein the initiation cycle comprises administering the bispecific antibody construct at a first dose of 36 μg once per day (e.g. daily) for 2 days, followed by administration of the bispecific antibody construct at a second dose of 72 μg once per day for 2 days, followed by administration of the bispecific antibody construct at a third dose of 110 μg once per day for 10 days, and wherein the maintenance cycle comprises administering the bispecific antibody construct at a dose of 110 μg once or twice every 7 days (e.g. once or twice per week) for 14 days, and wherein the maintenance cycle is administered the following day after completing the initiation cycle.

In yet another embodiment, the methods of the invention comprise administering to the patient at least one initiation cycle and at least one maintenance cycle of an anti-CD33×anti-CD3 bispecific antibody construct, wherein the initiation cycle comprises administering the bispecific antibody construct at a first dose of 18 μg once per day (e.g. daily) for 1 day, followed by administration of the bispecific antibody construct at a second dose of 36 μg once per day for 1 day, followed by administration of the bispecific antibody construct at a third dose of 72 μg once per day for 1 day, followed by administration of the bispecific antibody construct at a fourth dose of 110 μg once per day for 11 days, and wherein the maintenance cycle comprises administering the bispecific antibody construct at a dose of 110 μg once or twice every 7 days (e.g. once or twice per week) for 14 days, and wherein the maintenance cycle is administered the following day after completing the initiation cycle.

In still another embodiment, the methods of the invention comprise administering to the patient at least one initiation cycle and at least one maintenance cycle of an anti-CD33×anti-CD3 bispecific antibody construct, wherein the initiation cycle comprises administering the bispecific antibody construct at a first dose of 18 μg once per day (e.g. daily) for 1 day, followed by administration of the bispecific antibody construct at a second dose of 36 μg once per day for 1 day, followed by administration of the bispecific antibody construct at a third dose of 72 μg once per day for 1 day, followed by administration of the bispecific antibody construct at a fourth dose of 110 μg once per day for 1 day, followed by administration of the bispecific antibody construct at a fifth dose of 160 μg once per day for 10 days, and wherein the maintenance cycle comprises administering the bispecific antibody construct at a dose of 160 μg once or twice every 7 days (e.g. once or twice per week) for 14 days, and wherein the maintenance cycle is administered the following day after completing the initiation cycle.

In certain embodiments, the methods of the invention comprise administering to the patient at least one initiation cycle and at least one maintenance cycle of an anti-CD33×anti-CD3 bispecific antibody construct, wherein the initiation cycle comprises administering the bispecific antibody construct at a first dose of 18 μg once per day (e.g. daily) for 1 day, followed by administration of the bispecific antibody construct at a second dose of 36 μg once per day for 1 day, followed by administration of the bispecific antibody construct at a third dose of 72 μg once per day for 1 day, followed by administration of the bispecific antibody construct at a fourth dose of 110 μg once per day for 1 day, followed by administration of the bispecific antibody construct at a fifth dose of 160 μg once per day for 1 day, followed by administration of the bispecific antibody construct at a sixth dose of 240 μg once per day for 9 days, and wherein the maintenance cycle comprises administering the bispecific antibody construct at a dose of 240 μg once or twice every 7 days (e.g. once or twice per week) for 14 days, and wherein the maintenance cycle is administered the following day after completing the initiation cycle.

In other embodiments, the methods of the invention comprise administering to the patient at least one initiation cycle and at least one maintenance cycle of an anti-CD33×anti-CD3 bispecific antibody construct, wherein the initiation cycle comprises administering the bispecific antibody construct at a first dose of 18 μg once per day (e.g. daily) for 1 day, followed by administration of the bispecific antibody construct at a second dose of 36 μg once per day for 1 day, followed by administration of the bispecific antibody construct at a third dose of 72 μg once per day for 1 day, followed by administration of the bispecific antibody construct at a fourth dose of 110 μg once per day for 1 day, followed by administration of the bispecific antibody construct at a fifth dose of 160 μg once per day for 1 day, followed by administration of the bispecific antibody construct at a sixth dose of 240 μg once per day for 1 day, followed by administration of the bispecific antibody construct at a seventh dose of 360 μg once per day for 8 days, and wherein the maintenance cycle comprises administering the bispecific antibody construct at a dose of 360 μg once or twice every 7 days (e.g. once or twice per week) for 14 days, and wherein the maintenance cycle is administered the following day after completing the initiation cycle.

In some embodiments, the methods of the invention comprise administering to the patient at least one initiation cycle and at least one maintenance cycle of an anti-CD33×anti-CD3 bispecific antibody construct, wherein the initiation cycle comprises administering the bispecific antibody construct at a first dose of 18 μg once per day (e.g. daily) for 1 day, followed by administration of the bispecific antibody construct at a second dose of 36 μg once per day for 1 day, followed by administration of the bispecific antibody construct at a third dose of 72 μg once per day for 1 day, followed by administration of the bispecific antibody construct at a fourth dose of 110 μg once per day for 1 day, followed by administration of the bispecific antibody construct at a fifth dose of 160 μg once per day for 1 day, followed by administration of the bispecific antibody construct at a sixth dose of 240 μg once per day for 1 day, followed by administration of the bispecific antibody construct at a seventh dose of 360 μg once per day for 1 day, followed by administration of the bispecific antibody construct at an eighth dose of 480 μg once per day for 7 days, and wherein the maintenance cycle comprises administering the bispecific antibody construct at a dose of 480 μg once or twice every 7 days (e.g. once or twice per week) for 14 days, and wherein the maintenance cycle is administered the following day after completing the initiation cycle.

In any of the above-described embodiments or other embodiments described herein in which a step dosing regimen is employed during the initiation cycle, the step doses can be administered at more than one dosing interval (e.g. held constant for two or more dosing intervals) to delay the first administration of the target dose (i.e. the highest dose administered during the cycle). The duration of the steps can be different for each of the step doses such that a first dose is administered once at the first dosing interval and another step dose is administered at each of the next two or more dosing intervals. By way of illustration, in one embodiment, the methods of the invention comprise administering to the patient at least one initiation cycle and at least one maintenance cycle of an anti-CD33×anti-CD3 bispecific antibody construct, wherein the initiation cycle comprises administering the bispecific antibody construct at a first dose of 18 μg once per day (e.g. daily) for 1 day, followed by administration of the bispecific antibody construct at a second dose of 36 μg once per day for 2 days, followed by administration of the bispecific antibody construct at a third dose of 72 μg once per day for 2 days, followed by administration of the bispecific antibody construct at a fourth dose of 110 μg (e.g. target dose in this embodiment) once per day for 9 days, and wherein the maintenance cycle comprises administering the bispecific antibody construct at a dose of 110 μg once or twice every 7 days (e.g. once or twice per week) for 14 days, and wherein the maintenance cycle is administered the following day after completing the initiation cycle.

In certain embodiments, the methods of the invention comprise administering to the patient at least one initiation cycle and at least one maintenance cycle of an anti-CD33×anti-CD3 bispecific antibody construct, wherein the initiation cycle comprises administering the bispecific antibody construct at a first dose of 72 μg once per day (e.g. daily) for 2 days, followed by administration of the bispecific antibody construct at a second dose of 110 μg once per day for 12 days, and wherein the maintenance cycle comprises administering the bispecific antibody construct at a dose of 110 μg once or twice every 7 days (e.g. once or twice per week) for 14 days, and wherein the maintenance cycle is administered the following day after completing the initiation cycle.

In another embodiment, the methods of the invention comprise administering to the patient at least one initiation cycle and at least one maintenance cycle of an anti-CD33×anti-CD3 bispecific antibody construct, wherein the initiation cycle comprises administering the bispecific antibody construct at a first dose of 72 μg once per day (e.g. daily) for 2 days, followed by administration of the bispecific antibody construct at a second dose of 110 μg once per day for 2 days, followed by administration of the bispecific antibody construct at a third dose of 150 μg once per day for 10 days, and wherein the maintenance cycle comprises administering the bispecific antibody construct at a dose of 150 μg once or twice every 7 days (e.g. once or twice per week) for 14 days, and wherein the maintenance cycle is administered the following day after completing the initiation cycle.

In yet another embodiment, the methods of the invention comprise administering to the patient at least one initiation cycle and at least one maintenance cycle of an anti-CD33×anti-CD3 bispecific antibody construct, wherein the initiation cycle comprises administering the bispecific antibody construct at a first dose of 72 μg once per day (e.g. daily) for 2 days, followed by administration of the bispecific antibody construct at a second dose of 110 μg once per day for 2 days, followed by administration of the bispecific antibody construct at a third dose of 150 μg once per day for 2 days, followed by administration of the bispecific antibody construct at a fourth dose of 180 μg once per day for 8 days, and wherein the maintenance cycle comprises administering the bispecific antibody construct at a dose of 180 μg once or twice every 7 days (e.g. once or twice per week) for 14 days, and wherein the maintenance cycle is administered the following day after completing the initiation cycle.

In still another embodiment, the methods of the invention comprise administering to the patient at least one initiation cycle and at least one maintenance cycle of an anti-CD33×anti-CD3 bispecific antibody construct, wherein the initiation cycle comprises administering the bispecific antibody construct at a first dose of 72 μg once per day (e.g. daily) for 2 days, followed by administration of the bispecific antibody construct at a second dose of 110 μg once per day for 2 days, followed by administration of the bispecific antibody construct at a third dose of 150 μg once per day for 2 days, followed by administration of the bispecific antibody construct at a fourth dose of 180 μg once per day for 2 days, followed by administration of the bispecific antibody construct at a fifth dose of 240 μg once per day for 6 days, and wherein the maintenance cycle comprises administering the bispecific antibody construct at a dose of 240 μg once or twice every 7 days (e.g. once or twice per week) for 14 days, and wherein the maintenance cycle is administered the following day after completing the initiation cycle.

In certain embodiments, the methods of the invention comprise administering to the patient at least one initiation cycle and at least one maintenance cycle of an anti-CD33×anti-CD3 bispecific antibody construct, wherein the initiation cycle comprises administering the bispecific antibody construct at a first dose of 72 μg once per day (e.g. daily) for 2 days, followed by administration of the bispecific antibody construct at a second dose of 110 μg once per day for 2 days, followed by administration of the bispecific antibody construct at a third dose of 150 μg once per day for 2 days, followed by administration of the bispecific antibody construct at a fourth dose of 180 μg once per day for 2 days, followed by administration of the bispecific antibody construct at a fifth dose of 240 μg once per day for 2 days, followed by administration of the bispecific antibody construct at a sixth dose of 360 μg once per day for 4 days, and wherein the maintenance cycle comprises administering the bispecific antibody construct at a dose of 360 μg once or twice every 7 days (e.g. once or twice per week) for 14 days, and wherein the maintenance cycle is administered the following day after completing the initiation cycle.

In other embodiments, the methods of the invention comprise administering to the patient at least one initiation cycle and at least one maintenance cycle of an anti-CD33×anti-CD3 bispecific antibody construct, wherein the initiation cycle comprises administering the bispecific antibody construct at a first dose of 72 μg once per day (e.g. daily) for 2 days, followed by administration of the bispecific antibody construct at a second dose of 110 μg once per day for 2 days, followed by administration of the bispecific antibody construct at a third dose of 150 μg once per day for 2 days, followed by administration of the bispecific antibody construct at a fourth dose of 180 μg once per day for 2 days, followed by administration of the bispecific antibody construct at a fifth dose of 240 μg once per day for 2 days, followed by administration of the bispecific antibody construct at a sixth dose of 360 μg once per day for 2 days, followed by administration of the bispecific antibody construct at a seventh dose of 480 μg once per day for 2 days, and wherein the maintenance cycle comprises administering the bispecific antibody construct at a dose of 480 μg once or twice every 7 days (e.g. once or twice per week) for 14 days, and wherein the maintenance cycle is administered the following day after completing the initiation cycle.

In another embodiment, the methods of the invention comprise administering to the patient at least one initiation cycle and at least one maintenance cycle of an anti-CD33×anti-CD3 bispecific antibody construct, wherein the initiation cycle comprises administering the bispecific antibody construct at a first dose of 72 μg once per day (e.g. daily) for 2 days, followed by administration of the bispecific antibody construct at a second dose of 110 μg once per day for 2 days, followed by administration of the bispecific antibody construct at a third dose of 160 μg once per day for 10 days, and wherein the maintenance cycle comprises administering the bispecific antibody construct at a dose of 160 μg once or twice every 7 days (e.g. once or twice per week) for 14 days, and wherein the maintenance cycle is administered the following day after completing the initiation cycle.

In yet another embodiment, the methods of the invention comprise administering to the patient at least one initiation cycle and at least one maintenance cycle of an anti-CD33×anti-CD3 bispecific antibody construct, wherein the initiation cycle comprises administering the bispecific antibody construct at a first dose of 72 μg once per day (e.g. daily) for 2 days, followed by administration of the bispecific antibody construct at a second dose of 110 μg once per day for 2 days, followed by administration of the bispecific antibody construct at a third dose of 160 μg once per day for 2 days, followed by administration of the bispecific antibody construct at a fourth dose of 240 μg once per day for 8 days, and wherein the maintenance cycle comprises administering the bispecific antibody construct at a dose of 240 μg once or twice every 7 days (e.g. once or twice per week) for 14 days, and wherein the maintenance cycle is administered the following day after completing the initiation cycle.

In still another embodiment, the methods of the invention comprise administering to the patient at least one initiation cycle and at least one maintenance cycle of an anti-CD33×anti-CD3 bispecific antibody construct, wherein the initiation cycle comprises administering the bispecific antibody construct at a first dose of 72 μg once per day (e.g. daily) for 2 days, followed by administration of the bispecific antibody construct at a second dose of 110 μg once per day for 2 days, followed by administration of the bispecific antibody construct at a third dose of 160 μg once per day for 2 days, followed by administration of the bispecific antibody construct at a fourth dose of 240 μg once per day for 2 days, followed by administration of the bispecific antibody construct at a fifth dose of 360 μg once per day for 6 days, and wherein the maintenance cycle comprises administering the bispecific antibody construct at a dose of 360 μg once or twice every 7 days (e.g. once or twice per week) for 14 days, and wherein the maintenance cycle is administered the following day after completing the initiation cycle.

In some embodiments, the methods of the invention comprise administering to the patient at least one initiation cycle and at least one maintenance cycle of an anti-CD33×anti-CD3 bispecific antibody construct, wherein the initiation cycle comprises administering the bispecific antibody construct at a first dose of 72 μg once per day (e.g. daily) for 2 days, followed by administration of the bispecific antibody construct at a second dose of 110 μg once per day for 2 days, followed by administration of the bispecific antibody construct at a third dose of 160 μg once per day for 2 days, followed by administration of the bispecific antibody construct at a fourth dose of 240 μg once per day for 2 days, followed by administration of the bispecific antibody construct at a fifth dose of 360 μg once per day for 2 days, followed by administration of the bispecific antibody construct at a sixth dose of 480 μg once per day for 4 days, and wherein the maintenance cycle comprises administering the bispecific antibody construct at a dose of 480 μg once or twice every 7 days (e.g. once or twice per week) for 14 days, and wherein the maintenance cycle is administered the following day after completing the initiation cycle.

In certain embodiments of the methods of the invention, one or more premedications can be administered to the patient prior to the administration of a first dose of the bispecific antibody construct in the initiation cycle. In some embodiments, the premedication is administered to the patient prior to administration of each dose of the bispecific antibody construct in the initiation cycle. The premedication may also be administered to the patient prior to administration of one or more doses of the bispecific antibody construct in a maintenance cycle. In particular embodiments, the premedication is administered to the patient prior to administration of each dose of the bispecific antibody construct during the initiation cycle and one or more maintenance cycles—that is, the premedication is administered to the patient prior to administration of any dose of the bispecific antibody construct. It is envisaged that “prior to”, in this specific context, means within 24 hours, 18 hours, twelve hours, six hours, five hours, four hours, or three hours, and preferably within 120, 90, 60 or 30 minutes before the start of administration of the bispecific antibody construct. The premedication may e.g. be administered 30-120 or 30-60 minutes prior to start of administration of the bispecific antibody construct. The premedication may be administered e.g. to prevent or reduce severity of infusion-related reactions and/or to prevent or reduce severity of cytokine release syndrome or its symptoms.

In some embodiments, the premedication is an antihistamine. The antihistamine can be administered orally or intravenously and can be administered at a dose equivalent to diphenhydramine 50 mg i.v. Suitable antihistamines that can be administered as a premedication include, but are not limited to, antihistamines of oral, parenteral or rectal route such as: azatadine (maximum dose e.g. 4 mg/day), brompheniramine (maximum dose e.g. 30 mg/day), cetirizine (maximum dose e.g. 15 mg/day), chlorpheniramine (maximum dose e.g. 30 mg/day), clemastine (maximum dose e.g. 10 mg/day), cyproheptadine (maximum dose e.g. 15 mg/day), desloratadine (maximum dose e.g. 7 mg/day), dexchlorpheniramine (maximum dose e.g. 15 mg/day), diphenhydramine (maximum dose e.g. 350 mg/per day), doxylamine (maximum dose e.g. 180 mg/day), fexofenadine (maximum dose e.g. 200 mg/day), loratadine (maximum dose e.g. 15 mg/day), and phenindamine (maximum dose e.g. 180 mg/day).

In other embodiments, the premedication is a glucocorticoid. Glucocorticoids are a class of corticosteroids, which are a class of steroid hormones. Glucocorticoids are corticosteroids that bind to the glucocorticoid receptor. A less common synonym is glucocorticosteroid. Cortisol (known as hydrocortisone when used as a medication) is the most important human glucocorticoid. A variety of synthetic glucocorticoids, some far more potent than cortisol, have been created for therapeutic use. Cortisol is the standard of comparison for glucocorticoid potency. One example for commonly prescribed replacement steroid equivalents may be prednisone (5 mg)=cortisone (25 mg)=dexamethasone (0.75 mg)=hydrocortisone (20 mg)=methylprednisolone (4 mg). These doses indicate the equivalent pharmacologic dose of systemic glucocorticoids. The glucocorticoid can be administered orally or intravenously and can be administered at a dose equivalent to 4-20 mg dexamethasone i.v. (the equivalence referring to the glucocorticoid potency). The dose of glucocorticoid can be the same at each administration (i.e. at each time the glucocorticoid premedication is administered). Alternatively, the dose of glucocorticoid can be reduced in subsequent administrations, e.g. by 50% of the previous dose, if there are no or minimal signs of infusion reactions and/or CRS symptoms following the previous administration of the bispecific antibody construct.

Examples of glucocorticoids to be used as a premedication include, but are not limited to, cortisone, hydrocortisone, prednisone, prednisolone, methylprednisolone, dexamethasone, betamethasone, beclomethasone, budesonide, triamcinolone, cloprednol, deflazacort, fluocortolone, cortivazol, paramethasone, fluticasone, fluticasone propionate, triamcinolone acetonide, as well as combinations and/or pharmaceutically acceptable derivatives thereof. The different glucocorticoids may be used alone or in combination. Dexamethasone, prednisone and prednisolone are preferred glucocorticoids for use as a premedication according to the methods of the invention. In certain embodiments of the methods of the invention, the glucocorticoid administered to the patient prior to administration of one or more (or all) doses of the bispecific antibody construct during the initiation cycle and/or maintenance cycle is dexamethasone. Dexamethasone can be administered at a dose of about 4-20 mg, 6-18 mg, 8-16 mg, about 16 mg, or about 8 mg at each administration.

In certain embodiments, the premedication can be an IL-6 receptor antagonist, such as tocilizumab. Tocilizumab has been reported to effectively reduce or reverse symptoms of CRS induced by T cell-engaging therapies. See, e.g., Maude et al., Cancer J., Vol. 20:119-122, 2014. Tocilizumab can be administered at a dose of about 8 mg/kg to about 12 mg/kg body weight. Tocilizumab can be administered as a premedication once every week or once every two weeks. Thus, tocilizumab premedication need not be given before each dose of the bispecific antibody construct in the initiation cycle and/or maintenance cycle. By way of example, for an initiation and maintenance cycle of 14 days each, tocilizumab may be administered prior to the first dose of the bispecific antibody construct in the initiation cycle (e.g. on Day 1) and then administered prior to the first dose of the bispecific antibody construct in the first maintenance cycle (e.g. on Day 15).

A patient may be treated according to the methods of the invention for a set treatment period. A “treatment period” begins upon administration of a first dose of an anti-CD33×anti-CD3 bispecific antibody construct in an initiation cycle and ends upon administration of a final dose of an anti-CD33×anti-CD3 bispecific antibody construct in a maintenance cycle. The treatment period may be from about 3 months to about 36 months, from about 12 months to about 24 months, or from about 6 months to about 12 months. For instance, the treatment period may be about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, about 33 months, or about 36 months. In some embodiments, the treatment period is about 6 months. In other embodiments, the treatment period is about 9 months. In yet other embodiments, the treatment period is about 12 months. The treatment period can be adjusted for each patient depending on the patient's response to treatment. In one particular embodiment, the patient is treated according to the methods of the invention until the patient achieves complete remission or until leukemic disease is otherwise undetectable in the patient.

In certain embodiments, the patients to be treated according to the methods of the invention have previously received one or more prior treatments for acute myeloid leukemia, such as one or more chemotherapy regimens. Standard chemotherapy regimens for treating acute myeloid leukemia typically include a combination of cytarabine (also called cytosine arabinoside or ara-C) and an anthracycline, such as daunorubicin, doxorubicin, or idarubicin. Other treatments for acute myeloid leukemia include (i) midostaurin in combination with cytarabine and daunorubicin, (ii) venetoclax in combination with azacytidine or decitabine or low-dose cytarabine, and (iii) glasdegib in combination with low-dose cytarabine. In some embodiments, the patient to be treated according to the methods of the invention has failed to respond or is refractory to a prior therapy for acute myeloid leukemia. In certain embodiments, the patient to be treated according to the methods of the invention has failed to respond or is refractory to one or more chemotherapy regimens, particularly cytarabine-containing regimens. In other embodiments, the patient to be treated according to the methods of the invention has relapsed after treatment with one or more chemotherapy regimens, particularly cytarabine-containing regimens.

In certain embodiments, the patients to be treated according to the methods of the invention have previously received a hematopoietic stem cell transplant. The hematopoietic stem cell transplant may have been an allogeneic or autologous hematopoietic stem cell transplant. In one particular embodiment, the patient to be treated according to the methods of the invention has relapsed after having received a hematopoietic stem cell transplant.

The methods described herein comprise administering to a patient a bispecific antibody construct that specifically binds to CD33 and CD3. The term “antibody construct” refers to a molecule in which the structure and/or function is/are based on the structure and/or function of an antibody, e.g., of a full-length immunoglobulin molecule, or an antigen-binding fragment thereof. Accordingly, an antibody construct specifically binds to its target or antigen, and/or it comprises domains which are derived from or which are the heavy chain variable region (VH) and/or the light chain variable region (VL) of an antibody or fragment thereof. An antibody construct according to the invention generally comprises one or more binding domains, each of which will typically comprise the minimum structural requirements of an antibody that allow for specific target binding. This minimum requirement may, for example, be defined by the presence of at least three light chain “complementarity determining regions” or CDRs (i.e. CDRL1, CDRL2 and CDRL3 of a VL region) and/or three heavy chain CDRs (i.e. CDRH1, CDRH2 and CDRH3 of a VH region), and preferably all six CDRs from both the light and heavy chain variable regions. The antibodies on which the antibody constructs according to the invention are based include, for example, monoclonal, chimeric, humanized and human antibodies.

Preferably, the antibody constructs used in the methods of the invention are proteins and comprise one or more polypeptide chains. A polypeptide, as used herein, refers to a polymer of amino acids comprising at least 50 amino acids, preferably at least 100 amino acids. In some embodiments, the antibody constructs administered according to the methods of the invention are single-chain polypeptides. In other embodiments, the antibody constructs administered according to the methods of the invention comprise two or more polypeptide chains—e.g. are polypeptide dimers or multimers. In certain embodiments, the antibody constructs administered according to the methods of the invention comprise four polypeptide chains, and may, e.g. have the format of an antibody or an immunoglobulin protein.

As used herein, the term “antibody” generally refers to a tetrameric immunoglobulin protein comprising two light chain polypeptides (about 25 kDa each) and two heavy chain polypeptides (about 50-70 kDa each). The term “light chain” or “immunoglobulin light chain” refers to a polypeptide comprising, from amino terminus to carboxyl terminus, a single immunoglobulin light chain variable region (VL) and a single immunoglobulin light chain constant domain (CL). The immunoglobulin light chain constant domain (CL) can be a human kappa (κ) or human lambda (λ) constant domain. The term “heavy chain” or “immunoglobulin heavy chain” refers to a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu (μ), delta (Δ), gamma (γ), alpha (α), and epsilon (ε), and define the antibody's isotype as IgM, IgD, IgG, IgA, and IgE, respectively. The IgG-class and IgA-class antibodies are further divided into subclasses, namely, IgG1, IgG2, IgG3, and IgG4, and IgA1 and IgA2, respectively. The heavy chains in IgG, IgA, and IgD antibodies have three constant domains (CH1, CH2, and CH3), whereas the heavy chains in IgM and IgE antibodies have four constant domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (i.e. between the light and heavy chain) and between the hinge regions of the two antibody heavy chains.

Variable regions of immunoglobulin chains generally exhibit the same overall structure, comprising relatively conserved framework regions (FR) joined by three hypervariable regions, more often called “complementarity determining regions” or CDRs. The CDRs from the two chains of each heavy chain and light chain pair typically are aligned by the framework regions to form a structure that binds specifically to a specific epitope on the target protein (e.g., CD33 or CD3). From N-terminus to C-terminus, naturally-occurring light and heavy chain variable regions both typically conform with the following order of these elements: FR1, CDR1, FR2, CDR2, FR3, CDR3, and FR4. A numbering system has been devised for assigning numbers to amino acids that occupy positions in each of these domains. This numbering system is defined in Kabat Sequences of Proteins of Immunological Interest (1987 and 1991, NIH, Bethesda, Md.), or Chothia & Lesk, 1987, J. Mol. Biol. 196:901-917; Chothia et al., 1989, Nature 342:878-883. Complementarity determining regions (CDRs) and framework regions (FR) of a given antibody may be identified using this system. Other numbering systems for the amino acids in immunoglobulin chains include IMGT® (the international ImMunoGeneTics information system; Lefranc et al., Dev. Comp. Immunol. 29:185-203; 2005) and AH_{O} (Honegger and Pluckthun, J. Mol. Biol. 309(3):657-670; 2001).

The antibody constructs used in the methods of the invention are bispecific antibody constructs. The term “bispecific antibody construct” refers to a molecule capable of specifically binding to two different antigens. In the context of the present invention, such bispecific antibody constructs specifically bind to CD33 (e.g. human CD33) on the cell surface of target cells and CD3 (e.g. human CD3) on the cell surface of T cells. The term “anti-CD33×anti-CD3 bispecific antibody construct” is used herein to refer to a bispecific antibody construct that specifically binds to CD33 and CD3. An antibody construct or binding domain thereof “specifically binds” to a target antigen when it has a significantly higher binding affinity for, and consequently is capable of distinguishing, that antigen compared to its affinity for other unrelated proteins, under similar binding assay conditions. Antibody constructs or binding domains thereof that specifically bind an antigen may bind to that antigen with an equilibrium dissociation constant (K_{D})≤1×10⁻⁶ M. Antibody constructs or binding domains thereof specifically bind antigen with “high affinity” when the K_{D} is ≤1×10⁻⁸M. In one embodiment, the antibody constructs or binding domains thereof used in the methods of the invention bind to human CD33 and/or human CD3 with a K_{D} of ≤5×10⁻⁹M. In another embodiment, the antibody constructs or binding domains thereof used in the methods of the invention bind to human CD33 and/or human CD3 with a K_{D} of ≤1×10⁻⁹M. In yet another embodiment, the antibody constructs or binding domains thereof used in the methods of the invention bind to human CD33 and/or human CD3 with a K_{D} of ≤5×10⁻¹⁰ M. In another embodiment, the antibody constructs or binding domains thereof used in the methods of the invention bind to human CD33 and/or human CD3 with a K_{D} of ≤1×10⁻¹⁰ M. In certain embodiments, the antibody constructs or binding domains thereof used in the methods of the invention bind to human CD33 and/or human CD3 with a K_{D} of ≤5×10⁻¹¹ M. In other embodiments, the antibody constructs or binding domains thereof used in the methods of the invention bind to human CD33 and/or human CD3 with a K_{D} of ≤1×10⁻¹¹ M. In one particular embodiment, the antibody constructs or binding domains thereof used in the methods of the invention bind to human CD33 and/or human CD3 with a K_{D} of ≤5×10⁻¹² M. In another particular embodiment, the antibody constructs or binding domains thereof used in the methods of the invention bind to human CD33 and/or human CD3 with a K_{D} of ≤1×10⁻¹² M.

Affinity is determined using a variety of techniques, an example of which is an affinity ELISA assay. In various embodiments, affinity is determined by a surface plasmon resonance assay (e.g., BIAcore®-based assay). Using this methodology, the association rate constant (kₐ in M⁻¹s⁻¹) and the dissociation rate constant (k_{d} in s⁻¹) can be measured. The equilibrium dissociation constant (K_{D} in M) can then be calculated from the ratio of the kinetic rate constants (k_{d}/kₐ). In some embodiments, affinity is determined by a kinetic method, such as a Kinetic Exclusion Assay (KinExA) as described in Rathanaswami et al. Analytical Biochemistry, Vol. 373:52-60, 2008. Using a KinExA assay, the equilibrium dissociation constant (K_{D} in M) and the association rate constant (kₐ in M⁻¹s⁻¹) can be measured. The dissociation rate constant (k_{d} in s⁻¹) can be calculated from these values (K_{D}×kₐ). In other embodiments, affinity is determined by a bio-layer interferometry method, such as that described in Kumaraswamy et al., Methods Mol. Biol., Vol. 1278:165-82, 2015 and employed in Octet® systems (Pall ForteBio). The kinetic (kₐ and k_{d}) and affinity (K_{D}) constants can be calculated in real-time using the bio-layer interferometry method. In some embodiments, the antibody constructs or binding domains thereof described herein exhibit desirable characteristics such as binding avidity as measured by k_{d} (dissociation rate constant) for human CD33 and/or human CD3 of about 10⁻², 10⁻³, 10⁻⁴, 10⁻⁵, 10⁻⁶, 10⁻⁷, 10⁻⁸, 10⁻⁹, 10⁻¹⁰ s⁻¹ or lower (lower values indicating higher binding avidity), and/or binding affinity as measured by K_{D} (equilibrium dissociation constant) for human CD33 and/or human CD3 of about 10⁻⁸, 10⁻⁹, 10⁻¹⁰, 10⁻¹¹, 10⁻¹² M or lower (lower values indicating higher binding affinity).

In some embodiments, bispecific antibody constructs used in the methods of the invention may be antibodies and have the general structure of a full-length immunoglobulin. For example, the bispecific antibody constructs may comprise two full-length antibody heavy chains and two full-length antibody light chains. In particular embodiments, the bispecific antibody constructs are heterodimeric antibodies (used interchangeably herein with “hetero immunoglobulins” or “hetero Igs”), which refer to antibodies comprising two different light chains and two different heavy chains. For instance, in some embodiments, the heterodimeric antibody comprises a light chain and heavy chain from an anti-CD33 antibody and a light chain and heavy chain from an anti-CD3 antibody.

The bispecific antibody constructs employed in the methods of the invention may also comprise fragments of full-length antibodies, such as VH, VHH, VL, (s)dAb, Fv, light chain (VL-CL), Fd (VH-CH1), heavy chain, Fab, Fab′, F(ab′)2 or “r IgG” (“half antibody” consisting of a heavy chain and a light chain). Bispecific antibody constructs according to the invention may also comprise modified fragments of antibodies, also called antibody variants or antibody derivatives. Examples include, but are not limited to, single-chain variable fragment (scFv), di-scFv or bi(s)-scFv, scFv-Fc, scFv-zipper, single-chain Fab (scFab), Fab₂, Fab₃, diabodies, single chain diabodies, tandem diabodies (Tandab's), tandem di-scFv, tandem tri-scFv, “minibodies” exemplified by a structure which is as follows: (VH-VL-CH3)₂, (scFv-CH3)₂, ((scFv)₂-CH3+CH3), ((scFv)₂-CH3) or (scFv-CH3-scFv)₂, multibodies, such as triabodies or tetrabodies, and single domain antibodies, such as nanobodies or single variable domain antibodies comprising merely one variable region, which might be VHH, VH or VL, that specifically binds to an antigen or target independently of other variable regions or domains.

In certain embodiments, the bispecific antibody constructs used in the methods of the invention are multivalent. The valency of the antibody construct denotes the number of individual antigen-binding domains within the antibody construct. For example, the terms “monovalent,” “bivalent,” and “tetravalent” with reference to the antibody constructs in the context of the invention refer to antibody constructs with one, two, and four antigen-binding domains, respectively. Thus, a multivalent antibody construct comprises two or more antigen-binding domains. An antibody construct can have more antigen-binding domains (e.g. a higher valency) than specificities. For example, an antibody construct having two antigen-binding domains for a first target (e.g. CD33) and one antigen-binding domain for a second target (CD3)—or vice versa—is considered to be trivalent (three antigen-binding domains) and bispecific (binds to two antigens). In certain embodiments, the bispecific antibody constructs used in the methods of the invention are bivalent. Thus, such bispecific, bivalent antibody constructs contain two antigen binding domains: one antigen-binding domain for CD33 (e.g. human CD33) and one antigen-binding domain for CD3 (e.g. human CD3).

In some embodiments, the bispecific antibody constructs employed in the methods of the invention comprise a first binding domain that specifically binds to CD33 (e.g. human CD33) and a second binding domain that specifically binds to CD3 (e.g. human CD3). As used herein, the term “antigen-binding domain,” which is used interchangeably with “binding domain,” refers to the region of the antibody construct that contains the amino acid residues that interact with the antigen and confer on the antibody construct its specificity and affinity for the antigen. In certain embodiments, the binding domain of the antibody constructs may be derived from an antibody or antigen-binding fragment thereof. For instance, the binding domains of the bispecific antibody constructs used in the methods of the invention may comprise one or more complementarity determining regions (CDR) from the light and heavy chain variable regions of antibodies that specifically bind to human CD33 and/or human CD3. In some embodiments, the anti-CD33 binding domain of the bispecific antibody constructs comprises all six CDRs of the heavy and light chain variable regions of an anti-CD33 antibody described herein and the anti-CD3 binding domain of the bispecific antibody constructs comprises all six CDRs of the heavy and light chain variable regions of an anti-CD3 antibody described herein. In some embodiments, the binding domains (the anti-CD33 binding domain, the anti-CD3 binding domain or both) of the bispecific antibody constructs used in the methods of the invention comprise a Fab, a Fab′, a F(ab′)₂, a Fv, a single-chain variable fragment (scFv), or a nanobody. In one embodiment, both binding domains are Fab fragments. In another embodiment, one binding domain is a Fab fragment and the other binding domain is a scFv. In yet another embodiment, both binding domains are scFvs.

As used in the context of the invention, an “antigen-binding fragment,” used interchangeably herein with “binding fragment” or “fragment,” is a portion of an antibody that lacks at least some of the amino acids present in a full-length heavy chain and/or light chain, but which is still capable of specifically binding to an antigen. An antigen-binding fragment includes, but is not limited to, a single-chain variable fragment (scFv), a nanobody (e.g. VH domain of camelid heavy chain antibodies; VHH fragment, see Cortez-Retamozo et al., Cancer Research, Vol. 64:2853-57, 2004), a Fab fragment, a Fab′ fragment, a F(ab′)₂ fragment, a Fv fragment, a Fd fragment, and a complementarity determining region (CDR) fragment, and can be derived from any mammalian source, such as human, mouse, rat, rabbit, or camelid. Antigen-binding fragments may compete for binding of a target antigen with an intact antibody and the fragments may be produced by the modification of intact antibodies (e.g. enzymatic or chemical cleavage) or synthesized de novo using recombinant DNA technologies or peptide synthesis. In some embodiments, the antigen-binding fragment comprises at least one CDR from an antibody that binds to the antigen, for example, the heavy chain CDR3 from an antibody that binds to the antigen. In other embodiments, the antigen-binding fragment comprises all three CDRs from the heavy chain of an antibody that binds to the antigen or all three CDRs from the light chain of an antibody that binds to the antigen. In still other embodiments, the antigen-binding fragment comprises all six CDRs from an antibody that binds to the antigen (three from the heavy chain and three from the light chain).

Papain digestion of antibodies produces two identical antigen-binding fragments, called “Fab” fragments, each with a single antigen-binding site, and a residual “Fc” fragment which contains all but the first domain of the immunoglobulin heavy chain constant region. The Fab fragment contains the variable domains from the light and heavy chains, as well as the constant domain of the light chain and the first constant domain (CH1) of the heavy chain. Thus, a “Fab fragment” is comprised of one immunoglobulin light chain (light chain variable region (VL) and constant region (CL)) and the CH1 domain and variable region (VH) of one immunoglobulin heavy chain. The heavy chain of a Fab molecule cannot form a disulfide bond with another heavy chain molecule. The “Fd fragment” comprises the VH and CH1 domains from an immunoglobulin heavy chain. The Fd fragment represents the heavy chain component of the Fab fragment.

The “Fc fragment” or “Fc region” of an immunoglobulin generally comprises two constant domains, a CH2 domain and a CH3 domain, and optionally comprises a CH4 domain. In certain embodiments, the bispecific antibody constructs used in the methods of the invention comprise an Fc region from an immunoglobulin. The Fc region may be an Fc region from an IgG1, IgG2, IgG3, or IgG4 immunoglobulin. In some embodiments, the Fc region comprises CH2 and CH3 domains from a human IgG1 or human IgG2 immunoglobulin. The Fc region may retain effector function, such as C1q binding, complement dependent cytotoxicity (CDC), Fc receptor binding, antibody-dependent cell-mediated cytotoxicity (ADCC), and phagocytosis. In other embodiments, the Fc region may be modified to reduce or eliminate effector function.

A “Fab′ fragment” is a Fab fragment having at the C-terminus of the CH1 domain one or more cysteine residues from the antibody hinge region.

A “F(ab′)₂ fragment” is a bivalent fragment including two Fab′ fragments linked by a disulfide bridge between the heavy chains at the hinge region.

The “Fv” fragment is the minimum fragment that contains a complete antigen recognition and binding site from an antibody. This fragment consists of a dimer of one immunoglobulin heavy chain variable region (VH) and one immunoglobulin light chain variable region (VL) in tight, non-covalent association. It is in this configuration that the three CDRs of each variable region interact to define an antigen binding site on the surface of the VH-VL dimer. A single light chain or heavy chain variable region (or half of an Fv fragment comprising only three CDRs specific for an antigen) has the ability to recognize and bind antigen, although at a lower affinity than the entire binding site comprising both VH and VL.

A “single-chain variable fragment” or “scFv fragment” comprises the VH and VL regions of an antibody, wherein these regions are present in a single polypeptide chain, and optionally comprising a peptide linker between the VH and VL regions that enables the Fv to form the desired structure for antigen binding (see e.g., Bird et al., Science, Vol. 242:423-426, 1988; and Huston et al., Proc. Natl. Acad. Sci. USA, Vol. 85:5879-5883, 1988).

A “nanobody” is the heavy chain variable region of a heavy-chain antibody. Such variable domains are the smallest fully functional antigen-binding fragment of such heavy-chain antibodies with a molecular mass of only 15 kDa. See Cortez-Retamozo et al., Cancer Research 64:2853-57, 2004. Functional heavy-chain antibodies devoid of light chains are naturally occurring in certain species of animals, such as nurse sharks, wobbegong sharks and Camelidae, such as camels, dromedaries, alpacas and llamas. The antigen-binding site is reduced to a single domain, the VHH domain, in these animals. These antibodies form antigen-binding regions using only heavy chain variable region, i.e., these functional antibodies are homodimers of heavy chains only having the structure H₂L₂ (referred to as “heavy-chain antibodies” or “HCAbs”). Camelized VHH reportedly recombines with IgG2 and IgG3 constant regions that contain hinge, CH2, and CH3 domains and lack a CH1 domain. Camelized VHH domains have been found to bind to antigen with high affinity (Desmyter et al., J. Biol. Chem., Vol. 276:26285-90, 2001) and possess high stability in solution (Ewert et al., Biochemistry, Vol. 41:3628-36, 2002). Methods for generating antibodies having camelized heavy chains are described in, for example, U.S. Patent Publication Nos. 2005/0136049 and 2005/0037421. Alternative scaffolds can be made from human variable-like domains that more closely match the shark V-NAR scaffold and may provide a framework for a long penetrating loop structure.

In certain embodiments, the binding domains of the bispecific antibody constructs used in the methods of the invention comprise an immunoglobulin heavy chain variable region (VH) and an immunoglobulin light chain variable region (VL) of an antibody or antibody fragment which specifically binds to the desired antigen. For instance, the anti-CD33 binding domain of the bispecific antibody constructs of the invention comprises a VH region and VL region from an anti-CD33 antibody, such as any of the anti-CD33 antibodies or fragments thereof described herein, and the anti-CD3 binding domain comprises a VH region and VL region from an anti-CD3 antibody, such as any of the anti-CD3 antibodies or fragments thereof described herein. The binding domains that specifically bind to human CD33 or human CD3 can be derived from known antibodies to these antigens or from new antibodies or antibody fragments obtained by de novo immunization methods using the antigen proteins or fragments thereof, by phage display, or other methods described herein or known in the art. The antibodies from which the binding domains for the bispecific antibody constructs are derived can be monoclonal antibodies, recombinant antibodies, chimeric antibodies, human antibodies, or humanized antibodies. In certain embodiments, the antibodies from which the binding domains are derived are monoclonal antibodies. In these and other embodiments, the antibodies are human antibodies or humanized antibodies and can be of the IgG1-, IgG2-, IgG3-, or IgG4-type.

The first binding domain of the bispecific antibody constructs used in the methods of the invention specifically binds to CD33, preferably human CD33. This binding domain is referred to herein as an anti-CD33 binding domain. CD33 (cluster of differentiation 33; also known as sialic acid binding Ig-like lectin 3 (Siglec-3), gp67, or p6′7) is a transmembrane receptor expressed on the cells of myeloid lineage. More preferably, the first binding domain binds to CD33 on the surface of a target cell. The “target cell” can be any prokaryotic or eukaryotic cell expressing CD33 on its surface; preferably the target cell is a cell that is part of the human or animal body, such as a specific CD33-expressing cancer or tumor cell. It is furthermore envisaged that the first binding domain of the bispecific antibody constructs binds to human CD33, preferably to human CD33 on the surface of a target cell. It is also envisaged that the first binding domain binds to macaque CD33, preferably to macaque CD33 on the surface of a target cell. Exemplary amino acid sequences for the mature polypeptides and extracellular domains of human CD33 and macaque CD33 are provided in Table 1 below.

TABLE 1
Sequences of human and macaque CD33 polypeptides

| Polypeptide | Sequence |
|---|---|
| Human CD33 | DPNFWLQVQESVTVQEGLCVLVPCTFFHPIPYYDKN |
| | SPVHGYWFREGAIISRDSPVATNKLDQEVQEETQGR |
| | FRLLGDPSRNNCSLSIVDARRRDNGSYFFRMERGST |
| | KYSYKSPQLSVHVTDLTHRPKILIPGTLEPGHSKNL |
| | TCSVSWACEQGTPPIFSWLSAAPTSLGPRTTHSSVL |
| | IITPRPQDHGTNLTCQVKFAGAGVTTERTIQLNVTY |
| | VPQNPTTGIFPGDGSGKQETRAGVVHGAIGGAGVTA |
| | LLALCLCLIFFIVKTHRRKAARTAVGRNDTHPTTGS |
| | ASPKHQKKSKLHGPTETSSCSGAAPTVEMDEELHYA |
| | SLNFHGMNPSKDTSTEYSEVRTQ |
| | (SEQ ID NO: 1) |
| Macaque CD33 | DPRVRLEVQESVTVQEGLCVLVPCTFFHPVPYHTRN |
| | SPVHGYWFREGAIVSLDSPVATNKLDQEVQEETQGR |
| | FRLLGDPSRNNCSLSIVDARRRDNGSYFFRMEKGST |
| | KYSYKSTQLSVHVTDLTHRPQILIPGALDPDHSKNL |
| | TCSVPWACEQGTPPIFSWMSAAPTSLGLRTTHSSVL |
| | IITPRPQDHGTNLTCQVKFPGAGVTTERTIQLNVSY |
| | ASQNPRTDIFLGDGSGKQGVVQGAIGGAGVTVLLAL |
| | CLCLIFFTVKTHRRKAARTAVGRIDTHPATGPTSSK |
| | HQKKSKLHGATETSGCSGTTLTVEMDEELHYASLNF |
| | HGMNPSEDTSTEYSEVRTQ |
| | (SEQ ID NO: 2) |
| Extracellular | DPNFWLQVQESVTVQEGLCVLVPCTFFHPIPYYDKN |
| Domain of | SPVHGYWFREGAIISRDSPVATNKLDQEVQEETQGR |
| Human CD33 | FRLLGDPSRNNCSLSIVDARRRDNGSYFFRMERGST |
| | KYSYKSPQLSVHVTDLTHRPKILIPGTLEPGHSKNL |
| | TCSVSWACEQGTPPIFSWLSAAPTSLGPRTTHSSVL |
| | IITPRPQDHGTNLTCQVKFAGAGVTTERTIQLNVTY |
| | VPQNPTTGIFPGDGSGKQETRAGVVH |
| | (SEQ ID NO: 3) |
| Extracellular | DPRVRLEVQESVTVQEGLCVLVPCTFFHPVPYHTRN |
| Domain of | SPVHGYWFREGAIVSLDSPVATNKLDQEVQEETQGR |
| Macaque CD33 | FRLLGDPSRNNCSLSIVDARRRDNGSYFFRMEKGST |
| | KYSYKSTQLSVHVTDLTHRPQILIPGALDPDHSKNL |
| | TCSVPWACEQGTPPIFSWMSAAPTSLGLRTTHSSVL |
| | IITPRPQDHGTNLTCQVKFPGAGVTTERTIQLNVSY |
| | ASQNPRTDIFLGDGSGKQGVVQGAI |
| | (SEQ ID NO: 4) |

Examples of anti-CD33 binding domains from which the first binding domain of the bispecific antibody constructs used in the methods of the invention can be constructed or derived are described in WO 2008/119567, which is hereby incorporated by reference in its entirety. Light chain and heavy chain variable regions and associated CDRs of exemplary anti-human CD33 antibodies from which the anti-CD33 binding domain of the bispecific antibody constructs can be derived or constructed are set forth in Tables 2A and 2B, respectively.

TABLE 2A
Exemplary Anti-Human CD33 Antibody Light Chain
Variable Region Amino Acid Sequences

| | VL | | | | |
|---|---|---|---|---|---|
| Antibody ID. | Group | VL Amino Acid Sequence | CDRL1 | CDRL2 | CDRL3 |
| 01,02, 03, 04 | LV-01 | DIVMTQSPDSLTVSLGERTTIN | KSSQSVLDSSKNKNSLA | WASTRES | QQSAHFPIT |
| | | CKSSQSVLDSSKNKNSLAWYQQ | (SEQ ID NO: 5) | (SEQ ID NO: 8) | (SEQ ID NO: 9) |
| | | KPGQPPKLLLSWASTRESGIPD | | | |
| | | RFSGSGSGTDFTLTIDSLQPED | | | |
| | | SATYYCQQSAHFPITFGQGTRL | | | |
| | | EIK(SEQ ID NO: 15) | | | |
| 05 | LV-02 | DIVMTQSPDSMTVSLGERTTIN | KSSQSVLDSSTNKNSLA | WASTRES | QQSAHFPIT |
| | | CKSSQSVLDSSTNKNSLAWYQQ | (SEQ ID NO: 6) | (SEQ ID NO: 8) | (SEQ ID NO: 9) |
| | | KPGQPPKLLLSWASTRESGIPD | | | |
| | | RFSGSGSGTDFTLTIDSLQPED | | | |
| | | SATYYCQQSAHFPITFGQGTRL | | | |
| | | DIK(SEQ ID NO: 16) | | | |
| 06 | LV-03 | DIVMTQSPDSLSVSLGERTTIN | KSSQSVLDSSTNKNSLA | WASTRES | QQSAHFPIT |
| | | CKSSQSVLDSSTNKNSLAWYQQ | (SEQ ID NO: 6) | (SEQ ID NO: 8) | (SEQ ID NO: 9) |
| | | KPGQPPKLLLSWASTRESGIPD | | | |
| | | RFSGSGSGTDFTLTIDSLQPED | | | |
| | | SATYYCQQSAHFPITFGQGTRL | | | |
| | | EIK(SEQ ID NO: 17) | | | |
| 07 | LV-04 | DIVMTQSPDSLTVSLGERTTIN | KSSQSVLDSSNNKNSLA | WASTRES | QQSAHFPIT |
| | | CKSSQSVLDSSNNKNSLAWYQQ | (SEQ ID NO: 7) | (SEQ ID NO: 8) | (SEQ ID NO: 9) |
| | | KPGQPPKLLLSWASTRESGIPD | | | |
| | | RFSGSGSGTDFTLTIDGLQPED | | | |
| | | SATYYCQQSAHFPITFGQGTRL | | | |
| | | EIK(SEQ ID NO: 18) | | | |
| 08 | LV-05 | DIVMTQSPDSLTVSLGERTTIN | KSSQSVLDSSTNKNSLA | WASTRES | QQSAHFPIT |
| | | CKSSQSVLDSSTNKNSLAWYQQ | (SEQ ID NO: 6) | (SEQ ID NO: 8) | (SEQ ID NO: 9) |
| | | KPGQPPKLLLSWASTRESGIPD | | | |
| | | RFSGSGSGTDFTLTIDSPQPED | | | |
| | | SATYYCQQSAHFPITFGQGTRL | | | |
| | | EIK(SEQ ID NO: 19) | | | |
| 09 | LV-06 | DIVMTQSPDSLTVSLGERTTIN | KSSQSVLDSSTNKNSLA | WASTRES | QQSAHFPIT |
| | | CKSSQSVLDSSTNKNSLAWYQQ | (SEQ ID NO: 6) | (SEQ ID NO: 8) | (SEQ ID NO: 9) |
| | | KPGQPPKLLLSWASTRESGIPD | | | |
| | | RFSGSGSGTDFTLTIDSPQPED | | | |
| | | SATYYCQQSAHFPITFGCGTRL | | | |
| | | EIKS(SEQ ID NO: 20) | | | |

TABLE 2B
Exemplary Anti-Human CD33 Antibody Heavy Chain
Variable Region Amino Acid Sequences

| Antibody | VH | | | | |
|---|---|---|---|---|---|
| ID. | Group | VH Amino Acid Sequence | CDRH1 | CDRH2 | CDRH3 |
| 01 | HV-01 | QVQLVQSGAEVKKPGESVKVSC | NYGMN | WINTYTGEPTYADDFKG | WSWSDGYYVYFDY |
| | | KASGYTFTNYGMNWVRQAPGQG | (SEQ ID NO: 10) | (SEQ ID NO: 11) | (SEQ ID NO: 14) |
| | | LEWMGWINTYTGEPTYADDFKG | | | |
| | | RVTMSSDTSTSTAYLEINSLRS | | | |
| | | DDTAIYYCARWSWSDGYYVYFD | | | |
| | | YWGQGTTVTVSS | | | |
| | | (SEQ ID NO: 21) | | | |
| 02 | HV-02 | QVQLVQSGAEVKKPGASVKVSC | NYGMN | WINTYTGEPTYADDFKG | WSWSDGYYVYFDY |
| | | KASGYTFTNYGMNWVKQAPGQG | (SEQ ID NO: 10) | (SEQ ID NO: 11) | (SEQ ID NO: 14) |
| | | LKWMGWINTYTGEPTYADDFKG | | | |
| | | RVTMTSDTSTSTAYLELHNLRS | | | |
| | | DDTAVYYCARWSWSDGYYVYFD | | | |
| | | YWGQGTTVTVSS | | | |
| | | (SEQ ID NO: 22) | | | |
| 03 | HV-03 | QVQLVQSGAEVKKPGESVKVSC | NYGMN | WINTYTGEPTYADDFKG | WSWSDGYYVYFDY |
| | | KASGYTFTNYGMNWVKQAPGQG | (SEQ ID NO: 10) | (SEQ ID NO: 11) | (SEQ ID NO: 14) |
| | | LKWMGWINTYTGEPTYADDFKG | | | |
| | | RVTMTTDTSTSTAYMEIRNLRN | | | |
| | | DDTAVYYCARWSWSDGYYVYFD | | | |
| | | YWGQGTTVTVSS | | | |
| | | (SEQ ID NO: 23) | | | |
| 04 | HV-04 | QVQLVQSGAEVKKPGESVKVSC | NYGMN | WINTYTGEPTYADDFKG | WSWSDGYYVYFDY |
| | | KASGYTFTNYGMNWVKQAPGQG | (SEQ ID NO: 10) | (SEQ ID NO: 11) | (SEQ ID NO: 14) |
| | | LKWMGWINTYTGEPTYADDFKG | | | |
| | | RVTMTSDTSTSTAYMEISSLRS | | | |
| | | DDTAVYYCARWSWSDGYYVYFD | | | |
| | | YWGQGTTVTVSS | | | |
| | | (SEQ ID NO: 24) | | | |
| 05, 06 | HV-05 | QVQLVQSGAEVKKPGESVKVSC | NYGMN | WINTYTGETNYADKFQG | WSWSDGYYVYFDY |
| | | KASGYTFTNYGMNWVKQAPGQG | (SEQ ID NO: 10) | (SEQ ID NO: 12) | (SEQ ID NO: 14) |
| | | LEWMGWINTYTGETNYADKFQG | | | |
| | | RVTFTSDTSTSTAYMELRNLKS | | | |
| | | DDTAVYYCARWSWSDGYYVYFD | | | |
| | | YWGQGTTVTVSS | | | |
| | | (SEQ ID NO: 25) | | | |
| 07 | HV-06 | QVQLVQSGAEVKKPGESVKVSC | NYGMN | WINTYTGEPTYADKFQG | WSWSDGYYVYFDY |
| | | KASGYTFTNYGMNWVKQAPGQG | (SEQ ID NO: 10) | (SEQ ID NO: 13) | (SEQ ID NO: 14) |
| | | LEWMGWINTYTGEPTYADKFQG | | | |
| | | RVTMTTDTSTSTAYMEIRNLRS | | | |
| | | DDTAVYYCARWSWSDGYYVYFD | | | |
| | | YWGQGTTVTVSS | | | |
| | | (SEQ ID NO: 26) | | | |
| 08 | HV-07 | QVQLVQSGAEVKKPGESVKVSC | NYGMN | WINTYTGEPTYADKFQG | WSWSDGYYVYFDY |
| | | KASGYTFTNYGMNWVKQAPGQG | (SEQ ID NO: 10) | (SEQ ID NO: 13) | (SEQ ID NO: 14) |
| | | LEWMGWINTYTGEPTYADKFQG | | | |
| | | RVTMTTDTSTSTAYMEIRNLGG | | | |
| | | DDTAVYYCARWSWSDGYYVYFD | | | |
| | | YWGQGTSVTVSS | | | |
| | | (SEQ ID NO: 27) | | | |
| 09 | HV-08 | QVQLVQSGAEVKKPGESVKVSC | NYGMN | WINTYTGEPTYADKFQG | WSWSDGYYVYFDY |
| | | KASGYTFTNYGMNWVKQAPGQC | (SEQ ID NO: 10) | (SEQ ID NO: 13) | (SEQ ID NO: 14) |
| | | LEWMGWINTYTGEPTYADKFQG | | | |
| | | RVTMTTDTSTSTAYMEIRNLGG | | | |
| | | DDTAVYYCARWSWSDGYYVYFD | | | |
| | | YWGQGTSVTVSS | | | |
| | | (SEQ ID NO: 28) | | | |

The domain that specifically binds to human CD33 (e.g. the anti-CD33 binding domain) of the bispecific antibody constructs suitable for use in the methods of the invention may comprise one or more of the light chain CDRs (i.e. CDRLs) and/or heavy chain CDRs (i.e. CDRHs) presented in Tables 2A and 2B, respectively. For instance, in some embodiments, the anti-CD33 binding domains of the bispecific antibody constructs according to the invention comprise a CDRL1 comprising a sequence selected from SEQ ID NOs: 5 to 7; a CDRL2 comprising the sequence of SEQ ID NO: 8; a CDRL3 comprising the sequence of SEQ ID NO: 9; a CDRH1 comprising the sequence of SEQ ID NO: 10; a CDRH2 comprising a sequence selected from SEQ ID NOs: 11 to 13; and a CDRH3 comprising the sequence of SEQ ID NO: 14.

In some embodiments, the anti-CD33 binding domains of the bispecific antibody constructs comprise a light chain variable region comprising a CDRL1, a CDRL2, and a CDRL3, wherein: (a) CDRL1, CDRL2, and CDRL3 have the sequence of SEQ ID NOs: 5, 8 and 9, respectively; (b) CDRL1, CDRL2, and CDRL3 have the sequence of SEQ ID NOs: 6, 8 and 9, respectively; or (c) CDRL1, CDRL2, and CDRL3 have the sequence of SEQ ID NOs: 7, 8 and 9, respectively. In these and other embodiments, the anti-CD33 binding domains of the bispecific antibody constructs comprise a heavy chain variable region comprising a CDRH1, a CDRH2, and a CDRH3, wherein: (a) CDRH1, CDRH2, and CDRH3 have the sequence of SEQ ID NOs: 10, 11 and 14, respectively; (b) CDRH1, CDRH2, and CDRH3 have the sequence of SEQ ID NOs: 10, 12 and 14, respectively; or (c) CDRH1, CDRH2, and CDRH3 have the sequence of SEQ ID NOs: 10, 13 and 14, respectively.

In certain embodiments, the anti-CD33 binding domains of the bispecific antibody constructs suitable for use in the methods of the invention comprise a light chain variable region comprising a CDRL1, a CDRL2, and a CDRL3 and a heavy chain variable region comprising a CDRH1, a CDRH2, and a CDRH3, wherein:

(a) CDRL1, CDRL2, and CDRL3 have the sequence of SEQ ID NOs: 5, 8 and 9, respectively, and CDRH1, CDRH2, and CDRH3 have the sequence of SEQ ID NOs: 10, 11 and 14, respectively;

(b) CDRL1, CDRL2, and CDRL3 have the sequence of SEQ ID NOs: 6, 8 and 9, respectively, and CDRH1, CDRH2, and CDRH3 have the sequence of SEQ ID NOs: 10, 12 and 14, respectively;

(c) CDRL1, CDRL2, and CDRL3 have the sequence of SEQ ID NOs: 7, 8 and 9, respectively, and CDRH1, CDRH2, and CDRH3 have the sequence of SEQ ID NOs: 10, 13 and 14, respectively; or

(d) CDRL1, CDRL2, and CDRL3 have the sequence of SEQ ID NOs: 6, 8 and 9, respectively, and CDRH1, CDRH2, and CDRH3 have the sequence of SEQ ID NOs: 10, 13 and 14, respectively. In a preferred embodiment, the anti-CD33 binding domain of the bispecific antibody constructs used in the methods of the invention comprises (i) a light chain variable region comprising a CDRL1 having the sequence of SEQ ID NO: 6, a CDRL2 having the sequence of SEQ ID NO: 8, and a CDRL3 having the sequence of SEQ ID NO: 9, and (ii) a heavy chain variable region comprising a CDRH1 having the sequence of SEQ ID NO: 10, a CDRH2 having the sequence of SEQ ID NO: 13, and a CDRH3 having the sequence of SEQ ID NO: 14.

In some embodiments, the anti-CD33 binding domain of the bispecific antibody constructs used in the methods of the invention comprise an immunoglobulin heavy chain variable region (VH) and an immunoglobulin light chain variable region (VL) from an antibody that specifically binds to human CD33, such as the antibodies described herein. The “variable region,” used interchangeably herein with “variable domain” (variable region of a light chain (VL), variable region of a heavy chain (VH)), refers to the region in each of the light and heavy immunoglobulin chains which is involved directly in binding the antibody to the antigen. As discussed above, the regions of variable light and heavy chains have the same general structure and each region comprises four framework (FR) regions, the sequences of which are widely conserved, connected by three CDRs. The framework regions adopt a beta-sheet conformation and the CDRs may form loops connecting the beta-sheet structure. The CDRs in each chain are held in their three-dimensional structure by the framework regions and form, together with the CDRs from the other chain, the antigen binding site. Thus, in some embodiments, the anti-CD33 binding domain of the bispecific antibody constructs according to the invention may comprise a light chain variable region selected from LV-01 to LV-06 (SEQ ID NOs: 15-20), as shown in Table 2A, and/or a heavy chain variable region selected from HV-01 to HV-08 (SEQ ID NOs: 21-28), as shown in Table 2B, and binding fragments, derivatives, and variants of these light chain and heavy chain variable regions.

Each of the light chain variable regions listed in Table 2A may be combined with any of the heavy chain variable regions listed in Table 2B to form an anti-CD33 binding domain of the bispecific antibody constructs according to the invention. Examples of such combinations include, but are not limited to: (i) LV-01 and HV-01; (ii) LV-01 and HV-02; (iii) LV-01 and HV-03; (iv) LV-01 and HV-04; (v) LV-02 and HV-05; (vi) LV-03 and HV-05; (vii) LV-04 and HV-06; (viii) LV-05 and HV-07; and (ix) LV-06 and HV-08.

In certain embodiments, the anti-CD33 binding domains of the bispecific antibody constructs according to the invention comprise a light chain variable region comprising the sequence of SEQ ID NO: 15 and a heavy chain variable region comprising the sequence of SEQ ID NO: 21. In some embodiments, the anti-CD33 binding domains of the bispecific antibody constructs according to the invention comprise a light chain variable region comprising the sequence of SEQ ID NO: 15 and a heavy chain variable region comprising the sequence of SEQ ID NO: 22. In other embodiments, the anti-CD33 binding domains of the bispecific antibody constructs according to the invention comprise a light chain variable region comprising the sequence of SEQ ID NO: 15 and a heavy chain variable region comprising the sequence of SEQ ID NO: 23. In still other embodiments, the anti-CD33 binding domains of the bispecific antibody constructs according to the invention comprise a light chain variable region comprising the sequence of SEQ ID NO: 15 and a heavy chain variable region comprising the sequence of SEQ ID NO: 24. In some embodiments, the anti-CD33 binding domains of the bispecific antibody constructs according to the invention comprise a light chain variable region comprising the sequence of SEQ ID NO: 16 and a heavy chain variable region comprising the sequence of SEQ ID NO: 25. In certain embodiments, the anti-CD33 binding domains of the bispecific antibody constructs according to the invention comprise a light chain variable region comprising the sequence of SEQ ID NO: 17 and a heavy chain variable region comprising the sequence of SEQ ID NO: 25. In one embodiment, the anti-CD33 binding domains of the bispecific antibody constructs according to the invention comprise a light chain variable region comprising the sequence of SEQ ID NO: 18 and a heavy chain variable region comprising the sequence of SEQ ID NO: 26. In another embodiment, the anti-CD33 binding domains of the bispecific antibody constructs according to the invention comprise a light chain variable region comprising the sequence of SEQ ID NO: 19 and a heavy chain variable region comprising the sequence of SEQ ID NO: 27. In a preferred embodiment, the anti-CD33 binding domains of the bispecific antibody constructs according to the invention comprise a light chain variable region comprising the sequence of SEQ ID NO: 20 and a heavy chain variable region comprising the sequence of SEQ ID NO: 28.

In some embodiments, the anti-CD33 binding domains of the bispecific antibody constructs according to the invention comprise a light chain variable region comprising a sequence of contiguous amino acids that differs from the sequence of a light chain variable region in Table 2A, i.e. a VL selected from LV-01 to LV-06, at only 1, 2, 3, 4, 5, 6, 7, 8, 9, 10, 11, 12, 13, 14 or 15 amino acid residues, wherein each such sequence difference is independently either a deletion, insertion or substitution of one amino acid, with the deletions, insertions and/or substitutions resulting in no more than 15 amino acid changes relative to the foregoing variable domain sequences. The light chain variable region in some anti-CD33 binding domains comprises a sequence of amino acids that has at least 70%, at least 75%, at least 80%, at least 85%, at least 90%, at least 95%, at least 97% or at least 99% sequence identity to the amino acid sequences of SEQ ID NOs: 15 to 20 (i.e. the light chain variable regions in Table 2A).

In one embodiment, the anti-CD33 binding domains of the bispecific antibody constructs according to the invention comprise a light chain variable region comprising a sequence that is at least 90% identical to a sequence selected from SEQ ID NOs: 15-20. In another embodiment, the anti-CD33 binding domains of the bispecific antibody constructs according to the invention comprise a light chain variable region comprising a sequence that is at least 95% identical to a sequence selected from SEQ ID NOs: 15-20. In yet another embodiment, the anti-CD33 binding domains of the bispecific antibody constructs according to the invention comprise a light chain variable region comprising a sequence selected from SEQ ID NOs: 15-20.

In these and other embodiments, the anti-CD33 binding domains of the bispecific antibody constructs according to the invention comprise a heavy chain variable region comprising a sequence of contiguous amino acids that differs from the sequence of a heavy chain variable region in Table 2B, i.e., a VH selected from HV-01 to HV-08, at only 1, 2, 3, 4, 5, 6, 7, 8, 9, 10, 11, 12, 13, 14 or 15 amino acid residues, wherein each such sequence difference is independently either a deletion, insertion or substitution of one amino acid, with the deletions, insertions and/or substitutions resulting in no more than 15 amino acid changes relative to the foregoing variable domain sequences. The heavy chain variable region in some anti-CD33 binding domains comprises a sequence of amino acids that has at least 70%, at least 75%, at least 80%, at least 85%, at least 90%, at least 95%, at least 97% or at least 99% sequence identity to the amino acid sequences of SEQ ID NOs: 21 to 28 (i.e. the heavy chain variable regions in Table 2B).

In one embodiment, the anti-CD33 binding domains of the bispecific antibody constructs according to the invention comprise a heavy chain variable region comprising a sequence that is at least 90% identical to a sequence selected from SEQ ID NOs: 21-28. In another embodiment, the anti-CD33 binding domains of the bispecific antibody constructs according to the invention comprise a heavy chain variable region comprising a sequence that is at least 95% identical to a sequence selected from SEQ ID NOs: 21-28. In yet another embodiment, the anti-CD33 binding domains of the bispecific antibody constructs according to the invention comprise a heavy chain variable region comprising a sequence selected from SEQ ID NOs: 21-28.

The term “identity,” as used herein, refers to a relationship between the sequences of two or more polypeptide molecules or two or more nucleic acid molecules, as determined by aligning and comparing the sequences. “Percent identity,” as used herein, means the percent of identical residues between the amino acids or nucleotides in the compared molecules and is calculated based on the size of the smallest of the molecules being compared. For these calculations, gaps in alignments (if any) must be addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073. For example, sequence identity can be determined by standard methods that are commonly used to compare the similarity in position of the amino acids of two polypeptides. Using a computer program such as BLAST or FASTA, two polypeptide or two polynucleotide sequences are aligned for optimal matching of their respective residues (either along the full length of one or both sequences, or along a pre-determined portion of one or both sequences). The programs provide a default opening penalty and a default gap penalty, and a scoring matrix such as PAM 250 (Dayhoff et al., in Atlas of Protein Sequence and Structure, vol. 5, supp. 3, 1978) or BLOSUM62 (Henikoff et al., 1992, Proc. Natl. Acad. Sci. U.S.A. 89:10915-10919) can be used in conjunction with the computer program. For example, the percent identity can then be calculated as: the total number of identical matches multiplied by 100 and then divided by the sum of the length of the longer sequence within the matched span and the number of gaps introduced into the longer sequences in order to align the two sequences. In calculating percent identity, the sequences being compared are aligned in a way that gives the largest match between the sequences.

The GCG program package is a computer program that can be used to determine percent identity, which package includes GAP (Devereux et al., 1984, Nucl. Acid Res. 12:387; Genetics Computer Group, University of Wisconsin, Madison, Wis.). The computer algorithm GAP is used to align the two polypeptides or two polynucleotides for which the percent sequence identity is to be determined. The sequences are aligned for optimal matching of their respective amino acid or nucleotide (the “matched span,” as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Recommended parameters for determining percent identity for polypeptide or nucleotide sequences using the GAP program include the following:

Algorithm: Needleman et al. 1970, J. Mol. Biol. 48:443-453;

Comparison matrix: BLOSUM 62 from Henikoff et al., 1992, supra;

Gap Penalty: 12 (but with no penalty for end gaps)

Gap Length Penalty: 4

Threshold of Similarity: 0

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least 50 contiguous amino acids of the target polypeptide.

The second binding domain of the bispecific antibody constructs used in the methods of the invention specifically binds to CD3, preferably human CD3. This binding domain is referred to herein as an anti-CD3 binding domain. “CD3” (cluster of differentiation 3) is a T cell co-receptor composed of four chains. In mammals, the CD3 protein complex contains a CD3γ (gamma) chain, a CD3δ (delta) chain, and two CD3ε (epsilon) chains. These four chains associate with the T cell receptor (TCR) and the so-called ζ (zeta) chain to form the “T cell receptor complex” and to generate an activation signal in T lymphocytes. The CD3γ (gamma), CD3δ (delta), and CD3ε (epsilon) chains are highly related cell-surface proteins of the immunoglobulin superfamily and each contain a single extracellular immunoglobulin domain. The intracellular tails of the CD3 molecules contain a single conserved motif known as an immunoreceptor tyrosine-based activation motif (ITAM), which is essential for the signaling capacity of the TCR. The CD3 epsilon molecule is a polypeptide, which in humans is encoded by the CD3E gene which resides on chromosome 11.

The redirected lysis of target cells via the recruitment of T cells by an antibody construct which binds to CD3 on the T cell and to a target protein (e.g. CD33) on the target cell (e.g. myeloid cell) generally involves cytolytic synapse formation and delivery of perforin and granzymes. The engaged T cells are capable of serial target cell lysis, and are not affected by immune escape mechanisms interfering with peptide antigen processing and presentation, or clonal T cell differentiation; see, for example, WO 2007/042261.

In certain embodiments, the second binding domain of the bispecific antibody constructs according to the invention specifically binds to CD3 on the surface of a T cell, more preferably to human CD3 on the surface of a T cell. In some embodiments, the second binding domain of the bispecific antibody constructs specifically binds to CD3 epsilon, preferably human CD3 epsilon, e.g. human CD3 epsilon on the surface of a T cell. An exemplary amino acid sequence for the extracellular domain of human CD3 epsilon is provided below as SEQ ID NO: 29:

| (SEQ ID NO: 29) | |
|---|---|
| 1 | QDGNEEMGGI TQTPYKVSIS GTTVILTCPQ YPGSEILWQH NDKNIGGDED DKNIGSDEDH |
| 61 | LSLKEFSELE QSGYYVCYPR GSKPEDANFY LYLRARVCEN CMEMD |

Examples of anti-CD3 binding domains from which the second binding domain of the bispecific antibody constructs used in the methods of the invention can be constructed or derived are described in WO 2007/042261 and WO 2008/119567, both of which are hereby incorporated by reference in their entireties. Light chain and heavy chain variable regions and associated CDRs of exemplary anti-human CD3 antibodies from which the anti-CD3 binding domain of the bispecific antibody constructs can be derived or constructed are set forth in Tables 3A and 3B, respectively.

TABLE 3A
Exemplary Anti-Human CD3 Antibody Light
Chain Variable Region Amino Acid Sequences

| Antibody | VL | | | | |
|---|---|---|---|---|---|
| ID. | Group | VL Amino Acid Sequence | CDRL1 | CDRL2 | CDRL3 |
| 101, 102, | LV- | QTVVTQEPSLTVSPGGTVTLTC | GSSTGAVTSGYYPN | GTKFLAP | ALWYSNRWV |
| 103, 104, | 101 | GSSTGAVTSGYYPNWVQQKPGQ | (SEQ ID NO: 30) | (SEQ ID NO: 33) | (SEQ ID NO: 35) |
| 106, 108 | | APRGLIGGTKFLAPGTPARFSG | | | |
| | | SLLGGKAALTLSGVQPEDEAEY | | | |
| | | YCALWYSNRWVFGGGTKLTVL | | | |
| | | (SEQ ID NO: 57) | | | |
| 105, 107 | LV- | QTVVTQEPSLTVSPGGTVTLTC | RSSTGAVTSGYYPN | ATDMRPS | ALWYSNRWV |
| | 102 | RSSTGAVTSGYYPNWVQQKPGQ | (SEQ ID NO: 31) | (SEQ ID NO: 34) | (SEQ ID NO: 35) |
| | | APRGLIGATDMRPSGTPARFSG | | | |
| | | SLLGGKAALTLSGVQPEDEAEY | | | |
| | | YCALWYSNRWVFGGGTKLTVL | | | |
| | | (SEQ ID NO: 58) | | | |
| 109, 110 | LV- | QTVVTQEPSLTVSPGGTVTLTC | GSSTGAVTSGNYPN | GTKFLAP | VLWYSNRWV |
| | 103 | GSSTGAVTSGNYPNWVQQKPGQ | (SEQ ID NO: 32) | (SEQ ID NO: 33) | (SEQ ID NO: 36) |
| | | APRGLIGGTKFLAPGTPARFSG | | | |
| | | SLLGGKAALTLSGVQPEDEAEY | | | |
| | | YCVLWYSNRWVFGGGTKLTVL | | | |
| | | (SEQ ID NO: 59) | | | |

TABLE 3B
Exemplary Anti-Human CD3 Antibody Heavy Chain
Variable Region Amino Acid Sequences

| Antibody | VH | | | | |
|---|---|---|---|---|---|
| ID. | Group | VH Amino Acid Sequence | CDRH1 | CDRH2 | CDRH3 |
| 101 | HV- | EVQLVESGGGLVQPGGSLKLSC | IYAMN | RIRSKYNNYATYYADSVKS | HGNFGNSYVSFFAY |
| | 101 | AASGFTFNIYAMNWVRQAPGKG | (SEQ ID NO: 37) | (SEQ ID NO: 43) | (SEQ ID NO: 48) |
| | | LEWVARIRSKYNNYATYYADSV | | | |
| | | KSRFTISRDDSKNTAYLQMNNL | | | |
| | | KTEDTAVYYCVRHGNFGNSYVS | | | |
| | | FFAYWGQGTLVTVSS | | | |
| | | (SEQ ID NO: 60) | | | |
| 102, 110 | HV- | EVQLVESGGGLVQPGGSLKLSC | KYAMN | RIRSKYNNYATYYADSVKD | HGNFGNSYISYWAY |
| | 102 | AASGFTFNKYAMNWVRQAPGKG | (SEQ ID NO: 38) | (SEQ ID NO: 44) | (SEQ ID NO: 49) |
| | | LEWVARIRSKYNNYATYYADSV | | | |
| | | KDRFTISRDDSKNTAYLQMNNL | | | |
| | | KTEDTAVYYCVRHGNFGNSYIS | | | |
| | | YWAYWGQGTLVTVSS | | | |
| | | (SEQ ID NO: 61) | | | |
| 103 | HV- | EVQLVESGGGLEQPGGSLKLSC | SYAMN | RIRSKYNNYATYYADSVKG | HGNFGNSYLSFWAY |
| | 103 | AASGFTFNSYAMNWVRQAPGKG | (SEQ ID NO: 39) | (SEQ ID NO: 45) | (SEQ ID NO: 50) |
| | | LEWVARIRSKYNNYATYYADSV | | | |
| | | KGRFTISRDDSKNTAYLQMNNL | | | |
| | | KTEDTAVYYCVRHGNFGNSYLS | | | |
| | | FWAYWGQGTLVTVSS | | | |
| | | (SEQ ID NO: 62) | | | |
| 104 | HV- | EVQLVESGGGLVQPGGSLKLSC | RYAMN | RIRSKYNNYATYYADSVKG | HGNFGNSYLSYFAY |
| | 104 | AASGFTFNRYAMNWVRQAPGKG | (SEQ ID NO: 40) | (SEQ ID NO: 45) | (SEQ ID NO: 51) |
| | | LEWVARIRSKYNNYATYYADSV | | | |
| | | KGRFTISRDDSKNTAYLQMNNL | | | |
| | | KTEDTAVYYCVRHGNFGNSYLS | | | |
| | | YFAYWGQGTLVTVSS | | | |
| | | (SEQ ID NO: 63) | | | |
| 105 | HV- | EVQLVESGGGLVQPGGSLKLSC | VYAMN | RIRSKYNNYATYYADSVKK | HGNFGNSYLSWWAY |
| | 105 | AASGFTFNVYAMNWVRQAPGKG | (SEQ ID NO: 41) | (SEQ ID NO: 46) | (SEQ ID NO: 52) |
| | | LEWVARIRSKYNNYATYYADSV | | | |
| | | KKRFTISRDDSKNTAYLQMNNL | | | |
| | | KTEDTAVYYCVRHGNFGNSYLS | | | |
| | | WWAYWGQGTLVTVSS | | | |
| | | (SEQ ID NO: 64) | | | |
| 106 | HV- | EVQLVESGGGLVQPGGSLKLSC | KYAMN | RIRSKYNNYATYYADSVKS | HGNFGNSYTSYYAY |
| | 106 | AASGFTFNKYAMNWVRQAPGKG | (SEQ ID NO: 38) | (SEQ ID NO: 43) | (SEQ ID NO: 53) |
| | | LEWVARIRSKYNNYATYYADSV | | | |
| | | KSRFTISRDDSKNTAYLQMNNL | | | |
| | | KTEDTAVYYCVRHGNFGNSYTS | | | |
| | | YYAYWGQGTLVTVSS | | | |
| | | (SEQ ID NO: 65) | | | |
| 107 | HV- | EVQLVESGGGLVQPGGSLKLSC | GYAMN | RIRSKYNNYATYYADSVKE | HRNFGNSYLSWFAY |
| | 107 | AASGFTFNGYAMNWVRQAPGKG | (SEQ ID NO: 42) | (SEQ ID NO: 47) | (SEQ ID NO: 54) |
| | | LEWVARIRSKYNNYATYYADSV | | | |
| | | KERFTISRDDSKNTAYLQMNNL | | | |
| | | KTEDTAVYYCVRHR | | | |
| | | NFGNSYLSWFAYWGQGTLVT | | | |
| | | VSS(SEQ ID NO: 66) | | | |
| 108 | HV- | EVQLVESGGGLVQPGGSLKLS | VYAMN | RIRSKYNNYATYYADSVKK | HGNFGNSYISWWAY |
| | 108 | CAASGFTFNVYAMNWVRQAP | (SEQ ID NO: 41) | (SEQ ID NO: 46) | (SEQ ID NO: 55) |
| | | GKGLEWVARIRSKYNNYATY | | | |
| | | YADSVKKRFTISRDDSKNTAY | | | |
| | | LQMNNLKTEDTAVYYCVRHG | | | |
| | | NFGNSYISWWAYWGQGTLVT | | | |
| | | VSS(SEQ ID NO: 67) | | | |
| 109 | HV- | EVQLVESGGGLVQPGGSLKLS | SYAMN | RIRSKYNNYATYYADSVKG | HGNFGNSYVSWWAY |
| | 109 | CAASGFTFNSYAMNWVRQAP | (SEQ ID NO: 39) | (SEQ ID NO: 45) | (SEQ ID NO: 56) |
| | | GKGLEWVARIRSKYNNYATY | | | |
| | | YADSVKGRFTISRDDSKNTAY | | | |
| | | LQMNNLKTEDTAVYYCVRHG | | | |
| | | NFGNSYVSWWAYWGQGTLV | | | |
| | | TVSS(SEQ ID NO: 68) | | | |

The domain that specifically binds to human CD3 (e.g. the anti-CD3 binding domain) of the bispecific antibody constructs suitable for use in the methods of the invention may comprise one or more of the light chain CDRs (i.e. CDRLs) and/or heavy chain CDRs (i.e. CDRHs) presented in Tables 3A and 3B, respectively. For instance, in some embodiments, the anti-CD3 binding domains of the bispecific antibody constructs according to the invention comprise a CDRL1 comprising a sequence selected from SEQ ID NOs: 30 to 32; a CDRL2 comprising the sequence of SEQ ID NO: 33 or SEQ ID NO: 34; a CDRL3 comprising the sequence of SEQ ID NO: 35 or SEQ ID NO: 36; a CDRH1 comprising a sequence selected from SEQ ID NOs: 37 to 42; a CDRH2 comprising a sequence selected from SEQ ID NOs: 43 to 47; and a CDRH3 comprising a sequence selected from SEQ ID NOs: 48 to 56.

In some embodiments, the anti-CD3 binding domains of the bispecific antibody constructs comprise a light chain variable region comprising a CDRL1, a CDRL2, and a CDRL3, wherein: (a) CDRL1, CDRL2, and CDRL3 have the sequence of SEQ ID NOs: 30, 33 and 35, respectively; (b) CDRL1, CDRL2, and CDRL3 have the sequence of SEQ ID NOs: 31, 34 and 35, respectively; or (c) CDRL1, CDRL2, and CDRL3 have the sequence of SEQ ID NOs: 32, 33 and 36, respectively. In these and other embodiments, the anti-CD3 binding domains of the bispecific antibody constructs comprise a heavy chain variable region comprising a CDRH1, a CDRH2, and a CDRH3, wherein: (a) CDRH1, CDRH2, and CDRH3 have the sequence of SEQ ID NOs: 37, 43 and 48, respectively; (b) CDRH1, CDRH2, and CDRH3 have the sequence of SEQ ID NOs: 38, 44 and 49, respectively; (c) CDRH1, CDRH2, and CDRH3 have the sequence of SEQ ID NOs: 39, 45 and 50, respectively; (d) CDRH1, CDRH2, and CDRH3 have the sequence of SEQ ID NOs: 40, 45 and 51, respectively; (e) CDRH1, CDRH2, and CDRH3 have the sequence of SEQ ID NOs: 41, 46 and 52, respectively; (f) CDRH1, CDRH2, and CDRH3 have the sequence of SEQ ID NOs: 38, 43 and 53, respectively; (g) CDRH1, CDRH2, and CDRH3 have the sequence of SEQ ID NOs: 42, 47 and 54, respectively; (h) CDRH1, CDRH2, and CDRH3 have the sequence of SEQ ID NOs: 41, 46 and 55, respectively; or (i) CDRH1, CDRH2, and CDRH3 have the sequence of SEQ ID NOs: 39, 45 and 56, respectively.

In certain embodiments, the anti-CD3 binding domains of the bispecific antibody constructs suitable for use in the methods of the invention comprise a light chain variable region comprising a CDRL1, a CDRL2, and a CDRL3 and a heavy chain variable region comprising a CDRH1, a CDRH2, and a CDRH3, wherein:

(a) CDRL1, CDRL2, and CDRL3 have the sequence of SEQ ID NOs: 30, 33 and 35, respectively, and CDRH1, CDRH2, and CDRH3 have the sequence of SEQ ID NOs: 37, 43 and 48, respectively;

(b) CDRL1, CDRL2, and CDRL3 have the sequence of SEQ ID NOs: 30, 33 and 35, respectively, and CDRH1, CDRH2, and CDRH3 have the sequence of SEQ ID NOs: 38, 44 and 49, respectively;

(c) CDRL1, CDRL2, and CDRL3 have the sequence of SEQ ID NOs: 30, 33 and 35, respectively, and CDRH1, CDRH2, and CDRH3 have the sequence of SEQ ID NOs: 39, 45 and 50, respectively;

(d) CDRL1, CDRL2, and CDRL3 have the sequence of SEQ ID NOs: 30, 33 and 35, respectively, and CDRH1, CDRH2, and CDRH3 have the sequence of SEQ ID NOs: 40, 45 and 51, respectively;

(e) CDRL1, CDRL2, and CDRL3 have the sequence of SEQ ID NOs: 31, 34 and 35, respectively, and CDRH1, CDRH2, and CDRH3 have the sequence of SEQ ID NOs: 41, 46 and 52, respectively;

(f) CDRL1, CDRL2, and CDRL3 have the sequence of SEQ ID NOs: 30, 33 and 35, respectively, and CDRH1, CDRH2, and CDRH3 have the sequence of SEQ ID NOs: 38, 43 and 53, respectively;

(g) CDRL1, CDRL2, and CDRL3 have the sequence of SEQ ID NOs: 31, 34 and 35, respectively, and CDRH1, CDRH2, and CDRH3 have the sequence of SEQ ID NOs: 42, 47 and 54, respectively;

(h) CDRL1, CDRL2, and CDRL3 have the sequence of SEQ ID NOs: 30, 33 and 35, respectively, and CDRH1, CDRH2, and CDRH3 have the sequence of SEQ ID NOs: 41, 46 and 55, respectively;

(i) CDRL1, CDRL2, and CDRL3 have the sequence of SEQ ID NOs: 32, 33 and 36, respectively, and CDRH1, CDRH2, and CDRH3 have the sequence of SEQ ID NOs: 39, 45 and 56, respectively; or

(j) CDRL1, CDRL2, and CDRL3 have the sequence of SEQ ID NOs: 32, 33 and 36, respectively, and CDRH1, CDRH2, and CDRH3 have the sequence of SEQ ID NOs: 38, 44 and 49, respectively. In a preferred embodiment, the anti-CD3 binding domain of the bispecific antibody constructs used in the methods of the invention comprises (i) a light chain variable region comprising a CDRL1 having the sequence of SEQ ID NO: 32, a CDRL2 having the sequence of SEQ ID NO: 33, and a CDRL3 having the sequence of SEQ ID NO: 36, and (ii) a heavy chain variable region comprising a CDRH1 having the sequence of SEQ ID NO: 38, a CDRH2 having the sequence of SEQ ID NO: 44, and a CDRH3 having the sequence of SEQ ID NO: 49.

The anti-CD3 binding domain of the bispecific antibody constructs according to the invention may comprise a light chain variable region selected from LV-101 to LV-103 (SEQ ID NOs: 57-59), as shown in Table 3A, and/or a heavy chain variable region selected from HV-101 to HV-109 (SEQ ID NOs: 60-68), as shown in Table 3B, and binding fragments, derivatives, and variants of these light chain and heavy chain variable regions. Each of the light chain variable regions listed in Table 3A may be combined with any of the heavy chain variable regions listed in Table 3B to form an anti-CD3 binding domain of the bispecific antibody constructs according to the invention. Examples of such combinations include, but are not limited to: (i) LV-101 and HV-101; (ii) LV-101 and HV-102; (iii) LV-101 and HV-103; (iv) LV-101 and HV-104; (v) LV-101 and HV-106; (vi) LV-101 and HV-108; (vii) LV-102 and HV-105; (viii) LV-102 and HV-107; (ix) LV-103 and HV-109; and (x) LV-103 and HV-102.

In certain embodiments, the anti-CD3 binding domains of the bispecific antibody constructs according to the invention comprise a light chain variable region comprising the sequence of SEQ ID NO: 57 and a heavy chain variable region comprising the sequence of SEQ ID NO: 60. In some embodiments, the anti-CD3 binding domains of the bispecific antibody constructs according to the invention comprise a light chain variable region comprising the sequence of SEQ ID NO: 57 and a heavy chain variable region comprising the sequence of SEQ ID NO: 61. In other embodiments, the anti-CD3 binding domains of the bispecific antibody constructs according to the invention comprise a light chain variable region comprising the sequence of SEQ ID NO: 57 and a heavy chain variable region comprising the sequence of SEQ ID NO: 62. In still other embodiments, the anti-CD3 binding domains of the bispecific antibody constructs according to the invention comprise a light chain variable region comprising the sequence of SEQ ID NO: 57 and a heavy chain variable region comprising the sequence of SEQ ID NO: 63. In some embodiments, the anti-CD3 binding domains of the bispecific antibody constructs according to the invention comprise a light chain variable region comprising the sequence of SEQ ID NO: 58 and a heavy chain variable region comprising the sequence of SEQ ID NO: 64. In certain embodiments, the anti-CD3 binding domains of the bispecific antibody constructs according to the invention comprise a light chain variable region comprising the sequence of SEQ ID NO: 57 and a heavy chain variable region comprising the sequence of SEQ ID NO: 65. In one embodiment, the anti-CD3 binding domains of the bispecific antibody constructs according to the invention comprise a light chain variable region comprising the sequence of SEQ ID NO: 58 and a heavy chain variable region comprising the sequence of SEQ ID NO: 66. In another embodiment, the anti-CD3 binding domains of the bispecific antibody constructs according to the invention comprise a light chain variable region comprising the sequence of SEQ ID NO: 57 and a heavy chain variable region comprising the sequence of SEQ ID NO: 67. In a preferred embodiment, the anti-CD3 binding domains of the bispecific antibody constructs according to the invention comprise a light chain variable region comprising the sequence of SEQ ID NO: 59 and a heavy chain variable region comprising the sequence of SEQ ID NO: 61. In another preferred embodiment, the anti-CD3 binding domains of the bispecific antibody constructs according to the invention comprise a light chain variable region comprising the sequence of SEQ ID NO: 59 and a heavy chain variable region comprising the sequence of SEQ ID NO: 68.

In some embodiments, the anti-CD3 binding domains of the bispecific antibody constructs according to the invention comprise a light chain variable region comprising a sequence of contiguous amino acids that differs from the sequence of a light chain variable region in Table 3A, i.e. a VL selected from LV-101 to LV-103, at only 1, 2, 3, 4, 5, 6, 7, 8, 9, 10, 11, 12, 13, 14 or 15 amino acid residues, wherein each such sequence difference is independently either a deletion, insertion or substitution of one amino acid, with the deletions, insertions and/or substitutions resulting in no more than 15 amino acid changes relative to the foregoing variable domain sequences. The light chain variable region in some anti-CD3 binding domains comprises a sequence of amino acids that has at least 70%, at least 75%, at least 80%, at least 85%, at least 90%, at least 95%, at least 97% or at least 99% sequence identity to the amino acid sequences of SEQ ID NOs: 57 to 59 (i.e. the light chain variable regions in Table 3A).

In one embodiment, the anti-CD3 binding domains of the bispecific antibody constructs according to the invention comprise a light chain variable region comprising a sequence that is at least 90% identical to a sequence selected from SEQ ID NOs: 57-59. In another embodiment, the anti-CD3 binding domains of the bispecific antibody constructs according to the invention comprise a light chain variable region comprising a sequence that is at least 95% identical to a sequence selected from SEQ ID NOs: 57-59. In yet another embodiment, the anti-CD3 binding domains of the bispecific antibody constructs according to the invention comprise a light chain variable region comprising a sequence selected from SEQ ID NOs: 57-59.

In these and other embodiments, the anti-CD3 binding domains of the bispecific antibody constructs according to the invention comprise a heavy chain variable region comprising a sequence of contiguous amino acids that differs from the sequence of a heavy chain variable region in Table 3B, i.e., a VH selected from HV-101 to HV-109, at only 1, 2, 3, 4, 5, 6, 7, 8, 9, 10, 11, 12, 13, 14 or 15 amino acid residues, wherein each such sequence difference is independently either a deletion, insertion or substitution of one amino acid, with the deletions, insertions and/or substitutions resulting in no more than 15 amino acid changes relative to the foregoing variable domain sequences. The heavy chain variable region in some anti-CD3 binding domains comprises a sequence of amino acids that has at least 70%, at least 75%, at least 80%, at least 85%, at least 90%, at least 95%, at least 97% or at least 99% sequence identity to the amino acid sequences of SEQ ID NOs: 60 to 68 (i.e. the heavy chain variable regions in Table 3B).

In one embodiment, the anti-CD3 binding domains of the bispecific antibody constructs according to the invention comprise a heavy chain variable region comprising a sequence that is at least 90% identical to a sequence selected from SEQ ID NOs: 60-68. In another embodiment, the anti-CD3 binding domains of the bispecific antibody constructs according to the invention comprise a heavy chain variable region comprising a sequence that is at least 95% identical to a sequence selected from SEQ ID NOs: 60-68. In yet another embodiment, the anti-CD3 binding domains of the bispecific antibody constructs according to the invention comprise a heavy chain variable region comprising a sequence selected from SEQ ID NOs: 60-68.

According to certain embodiments, one or more of the binding domains of the bispecific antibody construct used in the methods of the invention, are in the format of an scFv. In an scFv, the VH region and the and VL region are arranged in the order VH-VL or VL-VH (from N- to C-terminus). It is envisaged that the VH and the VL regions of the first and/or the second binding domain are connected via a linker, preferably a peptide linker. In one embodiment of the first and/or second binding domain, the VH-region is positioned N-terminally of the linker, and the VL-region is positioned C-terminally of the linker. The linkers are preferably peptide linkers, more preferably short peptide linkers. Examples of suitable linkers include, but are not limited to:

(SEQ ID NO: 69)
GGGG
(SEQ ID NO: 70)
GGGGS
(SEQ ID NO: 71)
GGGGSGGGGS
(SEQ ID NO: 72)
GGGGSGGGGSGGGGS
(SEQ ID NO: 73)
GGGGSGGGGSGGGGSGGGGS
(SEQ ID NO: 74)
GGGGSGGGGSGGGGSGGGGSGGGGS
(SEQ ID NO: 75)
GGGGSGGGGSGGGGSGGGGSGGGGSGGGGS
(SEQ ID NO: 76)
GGGGSGGGGSGGGGSGGGGSGGGGSGGGGSGGGGS
(SEQ ID NO: 77)
GGGGSGGGGSGGGGSGGGGSGGGGSGGGGSGGGGSGGGGS
(SEQ ID NO: 78)
PGGGGS
(SEQ ID NO: 79)
PGGDGS
(SEQ ID NO: 80)
SGGGGS
(SEQ ID NO: 81)
GGGGSGGGS
(SEQ ID NO: 82)
GGGGQ

In the present context, a “short” linker has between 2 and 50 amino acids, preferably between 3 and 35, between 4 and 30, between 5 and 25, between 6 and 20 or between 6 and 17 amino acids. The linker between two variable regions of one binding domain may have a different length (e.g. may be longer) than the linker between the two binding domains. For example, the linker between two variable regions of one or both binding domains may have a length between 8 and 16 amino acids, preferably between 10 and 15, and the linker between the two binding domains may have a length between 3 and 10 amino acids, preferably between 5 and 8. It is further envisaged that the peptide linkers are glycine/serine linkers, such as those depicted in SEQ ID NOs: 70-81. In one embodiment, the anti-CD33 binding domain and/or the anti-CD3 binding domain of the bispecific antibody construct according to the invention is an scFv comprising, from N-terminus to C-terminus, a VH region—peptide linker—VL region, where the peptide linker comprises a glycine-serine linker, such as the linker set forth in SEQ ID NO: 72. In related embodiments, the peptide linker between the anti-CD33 and anti-CD3 binding domains (e.g. scFv domains) is the linker set forth in SEQ ID NO: 70 or SEQ ID NO: 80. Exemplary scFv domains for the anti-CD33 and anti-CD3 binding domains of the bispecific antibody constructs suitable for use in the methods of the invention are set forth in Table 4 below.

TABLE 4
Exemplary Single-Chain Variable Fragment Binding Domains

| | | |
|---|---|---|
| Designation | SEQ ID NO. | Amino Acid Sequence |

Anti-CD33 scFv domains

| | | |
|---|---|---|
| CD33 scFv-01 | 83 | QVQLVQSGAEVKKPGESVKVSCKASGYTFTNYGMNWVRQAPGQGLEWMGWINTYT |
| | | GEPTYADDFKGRVTMSSDTSTSTAYLEINSLRSDDTAIYYCARWSWSDGYYVYFD |
| | | YWGQGTTVTVSSGGGGSGGGGSGGGGSDIVMTQSPDSLTVSLGERTTINCKSSQS |
| | | VLDSSKNKNSLAWYQQKPGQPPKLLLSWASTRESGIPDRFSGSGSGTDFTLTIDS |
| | | LQPEDSATYYCQQSAHFPITFGQGTRLEIK |
| CD33 scFv-02 | 84 | QVQLVQSGAEVKKPGASVKVSCKASGYTFTNYGMNWVKQAPGQGLKWMGWINTYT |
| | | GEPTYADDFKGRVTMTSDTSTSTAYLELHNLRSDDTAVYYCARWSWSDGYYVYFD |
| | | YWGQGTTVTVSSGGGGSGGGGSGGGGSDIVMTQSPDSLTVSLGERTTINCKSSQS |
| | | VLDSSKNKNSLAWYQQKPGQPPKLLLSWASTRESGIPDRFSGSGSGTDFTLTIDS |
| | | LQPEDSATYYCQQSAHFPITFGQGTRLEIK |
| CD33 scFv-03 | 85 | QVQLVQSGAEVKKPGESVKVSCKASGYTFTNYGMNWVKQAPGQGLKWMGWINTYT |
| | | GEPTYADDFKGRVTMTTDTSTSTAYMEIRNLRNDDTAVYYCARWSWSDGYYVYFD |
| | | YWGQGTTVTVSSGGGGSGGGGSGGGGSDIVMTQSPDSLTVSLGERTTINCKSSQS |
| | | VLDSSKNKNSLAWYQQKPGQPPKLLLSWASTRESGIPDRFSGSGSGTDFTLTIDS |
| | | LQPEDSATYYCQQSAHFPITFGQGTRLEIK |
| CD33 scFv-04 | 86 | QVQLVQSGAEVKKPGESVKVSCKASGYTFTNYGMNWVKQAPGQGLKWMGWINTYT |
| | | GEPTYADDFKGRVTMTSDTSTSTAYMEISSLRSDDTAVYYCARWSWSDGYYVYFD |
| | | YWGQGTTVTVSSGGGGSGGGGSGGGGSDIVMTQSPDSLTVSLGERTTINCKSSQS |
| | | VLDSSKNKNSLAWYQQKPGQPPKLLLSWASTRESGIPDRFSGSGSGTDFTLTIDS |
| | | LQPEDSATYYCQQSAHFPITFGQGTRLEIK |
| CD33 scFv-05 | 87 | QVQLVQSGAEVKKPGESVKVSCKASGYTFTNYGMNWVKQAPGQGLEWMGWINTYT |
| | | GETNYADKFQGRVTFTSDTSTSTAYMELRNLKSDDTAVYYCARWSWSDGYYVYFD |
| | | YWGQGTTVTVSSGGGGSGGGGSGGGGSDIVMTQSPDSMTVSLGERTTINCKSSQS |
| | | VLDSSTNKNSLAWYQQKPGQPPKLLLSWASTRESGIPDRFSGSGSGTDFTLTIDS |
| | | LQPEDSATYYCQQSAHFPITFGQGTRLDIK |
| CD33 scFv-06 | 88 | QVQLVQSGAEVKKPGESVKVSCKASGYTFTNYGMNWVKQAPGQGLEWMGWINTYT |
| | | GETNYADKFQGRVTFTSDTSTSTAYMELRNLKSDDTAVYYCARWSWSDGYYVYFD |
| | | YWGQGTTVTVSSGGGGSGGGGSGGGGSDIVMTQSPDSLSVSLGERTTINCKSSQS |
| | | VLDSSTNKNSLAWYQQKPGQPPKLLLSWASTRESGIPDRFSGSGSGTDFTLTIDS |
| | | LQPEDSATYYCQQSAHFPITFGQGTRLEIK |
| CD33 scFv-07 | 89 | QVQLVQSGAEVKKPGESVKVSCKASGYTFTNYGMNWVKQAPGQGLEWMGWINTYT |
| | | GEPTYADKFQGRVTMTTDTSTSTAYMEIRNLRSDDTAVYYCARWSWSDGYYVYFD |
| | | YWGQGTTVTVSSGGGGSGGGGSGGGGSDIVMTQSPDSLTVSLGERTTINCKSSQS |
| | | VLDSSNNKNSLAWYQQKPGQPPKLLLSWASTRESGIPDRFSGSGSGTDFTLTIDG |
| | | LQPEDSATYYCQQSAHFPITFGQGTRLEIK |
| CD33 scFv-08 | 90 | QVQLVQSGAEVKKPGESVKVSCKASGYTFTNYGMNWVKQAPGQGLEWMGWINTYT |
| | | GEPTYADKFQGRVTMTTDTSTSTAYMEIRNLGGDDTAVYYCARWSWSDGYYVYFD |
| | | YWGQGTSVTVSSGGGGSGGGGSGGGGSDIVMTQSPDSLTVSLGERTTINCKSSQS |
| | | VLDSSTNKNSLAWYQQKPGQPPKLLLSWASTRESGIPDRFSGSGSGTDFTLTIDS |
| | | PQPEDSATYYCQQSAHFPITFGQGTRLEIK |
| CD33 scFv-09 | 91 | QVQLVQSGAEVKKPGESVKVSCKASGYTFTNYGMNWVKQAPGQCLEWMGWINTYT |
| | | GEPTYADKFQGRVTMTTDTSTSTAYMEIRNLGGDDTAVYYCARWSWSDGYYVYFD |
| | | YWGQGTSVTVSSGGGGSGGGGSGGGGSDIVMTQSPDSLTVSLGERTTINCKSSQS |
| | | VLDSSTNKNSLAWYQQKPGQPPKLLLSWASTRESGIPDRFSGSGSGTDFTLTIDS |
| | | PQPEDSATYYCQQSAHFPITFGCGTRLEIK |

Anti-CD3 scFv domains

| | | |
|---|---|---|
| CD3 scFv-01 | 92 | EVQLVESGGGLVQPGGSLKLSCAASGFTFNIYAMNWVRQAPGKGLEWVARIRSKY |
| | | NNYATYYADSVKSRFTISRDDSKNTAYLQMNNLKTEDTAVYYCVRHGNFGNSYVS |
| | | FFAYWGQGTLVTVSSGGGGSGGGGSGGGGSQTVVTQEPSLTVSPGGTVTLTCGSS |
| | | TGAVTSGYYPNWVQQKPGQAPRGLIGGTKFLAPGTPARFSGSLLGGKAALTLSGV |
| | | QPEDEAEYYCALWYSNRWVFGGGTKLTVL |
| CD3 scFv-02 | 93 | EVQLVESGGGLVQPGGSLKLSCAASGFTFNKYAMNWVRQAPGKGLEWVARIRSKY |
| | | NNYATYYADSVKDRFTISRDDSKNTAYLQMNNLKTEDTAVYYCVRHGNFGNSYIS |
| | | YWAYWGQGTLVTVSSGGGGSGGGGSGGGGSQTVVTQEPSLTVSPGGTVTLTCGSS |
| | | TGAVTSGYYPNWVQQKPGQAPRGLIGGTKFLAPGTPARFSGSLLGGKAALTLSGV |
| | | QPEDEAEYYCALWYSNRWVFGGGTKLTVL |
| CD3 scFv-03 | 94 | EVQLVESGGGLEQPGGSLKLSCAASGFTFNSYAMNWVRQAPGKGLEWVARIRSKY |
| | | NNYATYYADSVKGRFTISRDDSKNTAYLQMNNLKTEDTAVYYCVRHGNFGNSYLS |
| | | FWAYWGQGTLVTVSSGGGGSGGGGSGGGGSQTVVTQEPSLTVSPGGTVTLTCGSS |
| | | TGAVTSGYYPNWVQQKPGQAPRGLIGGTKFLAPGTPARFSGSLLGGKAALTLSGV |
| | | QPEDEAEYYCALWYSNRWVFGGGTKLTVL |
| CD3 scFv-04 | 95 | EVQLVESGGGLVQPGGSLKLSCAASGFTFNRYAMNWVRQAPGKGLEWVARIRSKY |
| | | NNYATYYADSVKGRFTISRDDSKNTAYLQMNNLKTEDTAVYYCVRHGNFGNSYLS |
| | | YFAYWGQGTLVTVSSGGGGSGGGGSGGGGSQTVVTQEPSLTVSPGGTVTLTCGSS |
| | | TGAVTSGYYPNWVQQKPGQAPRGLIGGTKFLAPGTPARFSGSLLGGKAALTLSGV |
| | | QPEDEAEYYCALWYSNRWVFGGGTKLTVL |
| CD3 scFv-05 | 96 | EVQLVESGGGLVQPGGSLKLSCAASGFTFNVYAMNWVRQAPGKGLEWVARIRSKY |
| | | NNYATYYADSVKKRFTISRDDSKNTAYLQMNNLKTEDTAVYYCVRHGNFGNSYLS |
| | | WWAYWGQGTLVTVSSGGGGSGGGGSGGGGSQTVVTQEPSLTVSPGGTVTLTCRSS |
| | | TGAVTSGYYPNWVQQKPGQAPRGLIGATDMRPSGTPARFSGSLLGGKAALTLSGV |
| | | QPEDEAEYYCALWYSNRWVFGGGTKLTVL |
| CD3 scFv-06 | 97 | EVQLVESGGGLVQPGGSLKLSCAASGFTFNKYAMNWVRQAPGKGLEWVARIRSKY |
| | | NNYATYYADSVKSRFTISRDDSKNTAYLQMNNLKTEDTAVYYCVRHGNFGNSYTS |
| | | YYAYWGQGTLVTVSSGGGGSGGGGSGGGGSQTVVTQEPSLTVSPGGTVTLTCGSS |
| | | TGAVTSGYYPNWVQQKPGQAPRGLIGGTKFLAPGTPARFSGSLLGGKAALTLSGV |
| | | QPEDEAEYYCALWYSNRWVFGGGTKLTVL |
| CD3 scFv-07 | 98 | EVQLVESGGGLVQPGGSLKLSCAASGFTFNGYAMNWVRQAPGKGLEWVARIRSKY |
| | | NNYATYYADSVKERFTISRDDSKNTAYLQMNNLKTEDTAVYYCVRHRNFGNSYLS |
| | | WFAYWGQGTLVTVSSGGGGSGGGGSGGGGSQTVVTQEPSLTVSPGGTVTLTCRSS |
| | | TGAVTSGYYPNWVQQKPGQAPRGLIGATDMRPSGTPARFSGSLLGGKAALTLSGV |
| | | QPEDEAEYYCALWYSNRWVFGGGTKLTVL |
| CD3 scFv-08 | 99 | EVQLVESGGGLVQPGGSLKLSCAASGFTFNVYAMNWVRQAPGKGLEWVARIRSKY |
| | | NNYATYYADSVKKRFTISRDDSKNTAYLQMNNLKTEDTAVYYCVRHGNFGNSYIS |
| | | WWAYWGQGTLVTVSSGGGGSGGGGSGGGGSQTVVTQEPSLTVSPGGTVTLTCGSS |
| | | TGAVTSGYYPNWVQQKPGQAPRGLIGGTKFLAPGTPARFSGSLLGGKAALTLSGV |
| | | QPEDEAEYYCALWYSNRWVFGGGTKLTVL |
| CD3 scFv-09 | 100 | EVQLVESGGGLVQPGGSLKLSCAASGFTFNSYAMNWVRQAPGKGLEWVARIRSKY |
| | | NNYATYYADSVKGRFTISRDDSKNTAYLQMNNLKTEDTAVYYCVRHGNFGNSYVS |
| | | WWAYWGQGTLVTVSSGGGGSGGGGSGGGGSQTVVTQEPSLTVSPGGTVTLTCGSS |
| | | TGAVTSGNYPNWVQQKPGQAPRGLIGGTKFLAPGTPARFSGSLLGGKAALTLSGV |
| | | QPEDEAEYYCVLWYSNRWVFGGGTKLTVL |
| CD3 scFv-10 | 101 | EVQLVESGGGLVQPGGSLKLSCAASGFTFNKYAMNWVRQAPGKGLEWVARIRSKY |
| | | NNYATYYADSVKDRFTISRDDSKNTAYLQMNNLKTEDTAVYYCVRHGNFGNSYIS |
| | | YWAYWGQGTLVTVSSGGGGSGGGGSGGGGSQTVVTQEPSLTVSPGGTVTLTCGSS |
| | | TGAVTSGNYPNWVQQKPGQAPRGLIGGTKFLAPGTPARFSGSLLGGKAALTLSGV |
| | | QPEDEAEYYCVLWYSNRWVFGGGTKLTVL |

In certain embodiments, the bispecific antibody constructs suitable for use in the methods of the invention comprise a first binding domain that specifically binds to human CD33 and has an amino acid sequence selected from any one of SEQ ID NOs: 83-91, and a second binding domain that specifically binds to human CD3 and has an amino acid sequence selected from any one of SEQ ID NOs: 92-101. In a preferred embodiment, the first binding domain of the bispecific antibody constructs comprises the amino acid sequence of SEQ ID NO: 91. In another preferred embodiment, the second binding domain of the bispecific antibody constructs comprises the amino acid sequence of SEQ ID NO: 101.

The bispecific antibody constructs according to the invention can comprise any of the anti-CD33 scFv binding domains set forth in Table 4 in combination with any of the anti-CD3 scFv binding domains set forth in Table 4. For instance, in some embodiments, the bispecific antibody constructs comprise an anti-CD33 scFv binding domain from Table 4 and an anti-CD3 scFv binding domain from Table 4, wherein the anti-CD33 scFv binding domain is connected to the anti-CD3 scFv binding domain through a peptide linker, such as the peptide linkers described herein. In certain embodiments, the bispecific antibody construct comprises, in amino to carboxyl order, an anti-CD33 scFv binding domain, a peptide linker, and an anti-CD3 scFv binding domain. In some such embodiments, the peptide linker comprises the sequence of SEQ ID NO: 70 or SEQ ID NO: 80.

The bispecific antibody constructs according to the invention may also comprise additional domains, which, e.g., can modulate the pharmacokinetic profile of the molecule. For instance, the bispecific antibody constructs may further comprise an immunoglobulin Fc region, a domain derived from serum albumin (e.g. human serum albumin), or an albumin-binding domain (e.g. comprising human albumin binding peptides), and/or be conjugated to polyethylene glycol chains to increase the serum half-life of the bispecific antibody construct. In certain embodiments, the bispecific antibody constructs used in the methods of the invention further comprise one or more immunoglobulin Fc regions. Each immunoglobulin Fc region or “Fc monomer” typically comprises at least a CH2 domain and a CH3 domain from an immunoglobulin molecule. The Fc monomer may comprise the CH2 and CH3 domains from an IgG1, IgG2, IgG3, or IgG4 immunoglobulin. As an example, the CH2 domain comprises amino acids 231 to 340 of an IgG1 immunoglobulin and the CH3 domain comprises amino acids 341 to 446 of an IgG1 immunoglobulin, where the amino acid numbering is according to the EU numbering system described in Edelman et al., Proc. Natl. Acad. USA, Vol. 63: 78-85 (1969) and Kabat et al., Sequences of Proteins of Immunological Interest, 5th Ed. Public Health Service, National Institutes of Health Publication No. 91-3242, Bethesda, Md. (1991). The boundaries of the CH2 and CH3 domains may vary slightly from one IgG isoform to another, but the CH2 and CH3 domains in IgG2, IgG3, and IgG4 can be ascertained by alignment with the CH2 and CH3 domains in IgG1.

In some embodiments, the Fc monomer may comprise an immunoglobulin hinge region or portion thereof. The immunoglobulin hinge region is typically the region defined by amino acids 216 to 231 (according to the EU numbering system) of IgG immunoglobulins. In certain embodiments, the Fc monomer comprises a hinge region from an IgG1 immunoglobulin or a portion thereof. In some such embodiments, the IgG1 hinge region comprises the amino acid sequence DKTHTCPPCP (SEQ ID NO: 102) or EPKSCDKTHTCPPCP (SEQ ID NO: 103). In other embodiments, the Fc monomer comprises an IgG2 hinge region having the sequence ERKCCVECPPCP (SEQ ID NO: 104), an IgG3 hinge region having the sequence ELKTPLDTTHTCPRCP (SEQ ID NO: 105), EPKSCDTPPPCPRCP (SEQ ID NO: 106), or ELKTPLGDTTHTCPRCP (SEQ ID NO: 107), or an IgG4 hinge region having the sequence ESKYGPPCPSCP (SEQ ID NO: 108). In certain embodiments, the Fc monomer comprises, in amino to carboxyl order, an immunoglobulin hinge region, an immunoglobulin CH2 domain, and an immunoglobulin CH3 domain.

In certain embodiments, the bispecific antibody constructs comprise a domain having one Fc monomer. In alternative embodiments, the bispecific antibody constructs comprise a domain having two or more Fc monomers. For instance, in one embodiment, the bispecific antibody constructs used in the methods of the invention comprise a domain having two Fc monomers. The two Fc monomers can be present on separate polypeptide chains and associate to form a dimer, e.g. via non-covalent interactions and/or disulfide bonds (e.g. between cysteine residues in the hinge regions of Fc monomers). In a preferred embodiment, the two Fc monomers are fused to each other via a peptide linker, preferably a linker sufficient in length to allow the Fc monomers to associate and form an intra-chain dimer. The fusion of two Fc monomers to form a single polypeptide chain is referred to herein as a single-chain Fc domain (scFc domain) and is described in more detail below.

The peptide linker, by which the Fc monomers are fused to each other to form a single-chain Fc domain, preferably comprises at least 25 amino acid residues (e.g. 25, 26, 27, 28, 29, 30 or more). More preferably, this peptide linker comprises at least 30 amino acid residues (e.g. 30, 31, 32, 33, 34, 35 or more). In some embodiments, the linker comprises up to 40 amino acid residues, more preferably up to 35 amino acid residues, and even more preferably exactly 30 amino acid residues. In certain embodiments, the peptide linker comprises glycine-serine residues, for example repeats of the amino acid sequence Gly-Gly-Gly-Gly-Ser (SEQ ID NO: 70). In such embodiments, the peptide linker comprises (Gly₄Ser)ₓ, where x is an integer of 5 or greater (e.g. 6, 7 or 8). Preferably the integer is 6 or 7, more preferably the integer is 6. In one particular embodiment, the peptide linker used to connect the two Fc monomers to form a single-chain Fc domain comprises the sequence of SEQ ID NO: 75.

The Fc monomer may contain one or more amino acid substitutions relative to the native CH2 or CH3 immunoglobulin amino acid sequences, e.g. to modulate effector function, alter glycosylation, or enhance stability. For instance, in one embodiment, the glycosylation site in the CH2 domain at amino acid position 297 according to EU numbering is removed by substituting a different amino acid for the asparagine residue at this position. A N297G substitution is preferred in some embodiments. Stability enhancing mutations include the substitution of one or more amino acids in the CH2 and/or CH3 domains with cysteine residues to promote disulfide bond formation. Preferably, specific pairs of residues are substituted with cysteine such that they preferentially form a disulfide bond with each other, thus limiting or preventing disulfide bond scrambling. Preferred pairs include, but are not limited to, A287C and L306C, V259C and L306C, R292C and V302C, and V323C and I332C, with the amino acid positions numbered according to the EU numbering system. In one particular embodiment, the Fc monomer(s) incorporated into the third domain of the bispecific antibody constructs comprises N297G, R292C, and V302C substitutions, with the amino acid positions numbered according to the EU numbering system.

In certain preferred embodiments, the bispecific antibody constructs used in the methods of the invention comprise a third domain, which is a single-chain Fc domain. Accordingly, in certain such embodiments, the third domain comprises two Fc monomers, each monomer comprising an immunoglobulin hinge region, an immunoglobulin CH2 domain, and an immunoglobulin CH3 domain, wherein the two Fc monomers are fused to each other via a peptide linker as described herein. Exemplary amino acid sequences for the Fc monomers and the single-chain Fc (scFc) domains are provided in Table 5 below. In some embodiments, each of the Fc monomers of the third domain has an amino acid sequence that is at least 90% identical to a sequence selected from SEQ ID NOs: 109-116. In other embodiments, each of the Fc monomers of the third domain has an amino acid sequence selected from SEQ ID NOs: 109-116. In a preferred embodiment, each of the Fc monomers of the third domain comprises the amino acid sequence of SEQ ID NO: 109. In another preferred embodiment, each of the Fc monomers of the third domain comprises the amino acid sequence of SEQ ID NO: 110.

TABLE 5
Exemplary Fc Monomer and Single-Chain Fc Domains

| | | |
|---|---|---|
| Designation | SEQ ID NO. | Amino Acid Sequence |

Fc Monomers

| | | |
|---|---|---|
| Fc monomer −1 | 109 | DKTHTCPPCPAPELLGGPSVFLFPPKPKDTLMISRTPEVTCVVVDVSHEDPE |
| | | VKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFSCSVM |
| | | HEALHNHYTQKSLSLSPGK |
| Fc monomer −2 | 110 | DKTHTCPPCPAPELLGGPSVFLFPPKPKDTLMISRTPEVTCVVVDVSHEDPE |
| | | VKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFSCSVM |
| | | HEALHNHYTQKSLSLSP |
| Fc monomer −3 | 111 | DKTHTCPPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFSCSVM |
| | | HEALHNHYTQKSLSLSPGK |
| Fc monomer −4 | 112 | DKTHTCPPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFSCSVM |
| | | HEALHNHYTQKSLSLSP |
| Fc monomer −5 | 113 | DKTHTCPPCPAPELLGGPSVFLFPPKPKDTLMISRTPEVTCVVVDVSHEDPE |
| | | VKFNWYVDGVEVHNAKTKPREEQYGSTYRVVSVLTVLHQDWLNGKEYKCKVS |
| | | NKALPAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFSCSVM |
| | | HEALHNHYTQKSLSLSPGK |
| Fc monomer −6 | 114 | DKTHTCPPCPAPELLGGPSVFLFPPKPKDTLMISRTPEVTCVVVDVSHEDPE |
| | | VKFNWYVDGVEVHNAKTKPREEQYGSTYRVVSVLTVLHQDWLNGKEYKCKVS |
| | | NKALPAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFSCSVM |
| | | HEALHNHYTQKSLSLSP |
| Fc monomer −7 | 115 | DKTHTCPPCPAPELLGGPSVFLFPPKPKDTLMISRTPEVTCVVVDVSHEDPE |
| | | VKFNWYVDGVEVHNAKTKPCEEQYNSTYRCVSVLTVLHQDWLNGKEYKCKVS |
| | | NKALPAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFSCSVM |
| | | HEALHNHYTQKSLSLSPGK |
| Fc monomer −8 | 116 | DKTHTCPPCPAPELLGGPSVFLFPPKPKDTLMISRTPEVTCVVVDVSHEDPE |
| | | VKFNWYVDGVEVHNAKTKPCEEQYNSTYRCVSVLTVLHQDWLNGKEYKCKVS |
| | | NKALPAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFSCSVM |
| | | HEALHNHYTQKSLSLSP |

Single-Chain Fc Domains

| | | |
|---|---|---|
| scFc-1 | 117 | DKTHTCPPCPAPELLGGPSVFLFPPKPKDTLMISRTPEVTCVVVDVSHEDPE |
| | | VKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFSCSVM |
| | | HEALHNHYTQKSLSLSPGKGGGGSGGGGSGGGGSGGGGSGGGGSGGGGSDKT |
| | | HTCPPCPAPELLGGPSVFLFPPKPKDTLMISRTPEVTCVVVDVSHEDPEVKF |
| | | NWYVDGVEVHNAKTKPCEEQYGSTYRCVSVLTVLHQDWLNGKEYKCKVSNKA |
| | | LPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHEA |
| | | LHNHYTQKSLSLSPGK |
| scFc-2 | 118 | DKTHTCPPCPAPELLGGPSVFLFPPKPKDTLMISRTPEVTCVVVDVSHEDPE |
| | | VKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFSCSVM |
| | | HEALHNHYTQKSLSLSPGGGGSGGGGSGGGGSGGGGSGGGGSGGGGSDKTHT |
| | | CPPCPAPELLGGPSVFLFPPKPKDTLMISRTPEVTCVVVDVSHEDPEVKFNW |
| | | YVDGVEVHNAKTKPCEEQYGSTYRCVSVLTVLHQDWLNGKEYKCKVSNKALP |
| | | APIEKTISKAKGQPREPQVYTLPPSREEMTKNQVSLTCLVKGFYPSDIAVEW |
| | | ESNGQPENNYKTTPPVLDSDGSFFLYSKLTVDKSRWQQGNVFSCSVMHEALH |
| | | NHYTQKSLSLSP |
| scFc-3 | 119 | DKTHTCPPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFSCSVM |
| | | HEALHNHYTQKSLSLSPGKGGGGSGGGGSGGGGSGGGGSGGGGSGGGGSDKT |
| | | HTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNKA |
| | | LPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHEA |
| | | LHNHYTQKSLSLSPGK |
| scFc-4 | 120 | DKTHTCPPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFSCSVM |
| | | HEALHNHYTQKSLSLSPGGGGSGGGGSGGGGSGGGGSGGGGSGGGGSDKTHT |
| | | CPPCPAPELLGGPSVFLFPPKPKDTLMISRTPEVTCVVVDVSHEDPEVKFNW |
| | | YVDGVEVHNAKTKPREEQYNSTYRVVSVLTVLHQDWLNGKEYKCKVSNKALP |
| | | APIEKTISKAKGQPREPQVYTLPPSREEMTKNQVSLTCLVKGFYPSDIAVEW |
| | | ESNGQPENNYKTTPPVLDSDGSFFLYSKLTVDKSRWQQGNVFSCSVMHEALH |
| | | NHYTQKSLSLSP |
| scFc-5 | 121 | DKTHTCPPCPAPELLGGPSVFLFPPKPKDTLMISRTPEVTCVVVDVSHEDPE |
| | | VKFNWYVDGVEVHNAKTKPREEQYGSTYRVVSVLTVLHQDWLNGKEYKCKVS |
| | | NKALPAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFSCSVM |
| | | HEALHNHYTQKSLSLSPGKGGGGSGGGGSGGGGSGGGGSGGGGSGGGGSDKT |
| | | HTCPPCPAPELLGGPSVFLFPPKPKDTLMISRTPEVTCVVVDVSHEDPEVKF |
| | | NWYVDGVEVHNAKTKPREEQYGSTYRVVSVLTVLHQDWLNGKEYKCKVSNKA |
| | | LPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHEA |
| | | LHNHYTQKSLSLSPGK |
| scFc-6 | 122 | DKTHTCPPCPAPELLGGPSVFLFPPKPKDTLMISRTPEVTCVVVDVSHEDPE |
| | | VKFNWYVDGVEVHNAKTKPREEQYGSTYRVVSVLTVLHQDWLNGKEYKCKVS |
| | | NKALPAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFSCSVM |
| | | HEALHNHYTQKSLSLSPGGGGSGGGGSGGGGSGGGGSGGGGSGGGGSDKTHT |
| | | CPPCPAPELLGGPSVFLFPPKPKDTLMISRTPEVTCVVVDVSHEDPEVKFNW |
| | | YVDGVEVHNAKTKPREEQYGSTYRVVSVLTVLHQDWLNGKEYKCKVSNKALP |
| | | APIEKTISKAKGQPREPQVYTLPPSREEMTKNQVSLTCLVKGFYPSDIAVEW |
| | | ESNGQPENNYKTTPPVLDSDGSFFLYSKLTVDKSRWQQGNVFSCSVMHEALH |
| | | NHYTQKSLSLSP |
| scFc-7 | 123 | DKTHTCPPCPAPELLGGPSVFLFPPKPKDTLMISRTPEVTCVVVDVSHEDPE |
| | | VKFNWYVDGVEVHNAKTKPCEEQYNSTYRCVSVLTVLHQDWLNGKEYKCKVS |
| | | NKALPAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFSCSVM |
| | | HEALHNHYTQKSLSLSPGKGGGGSGGGGSGGGGSGGGGSGGGGSGGGGSDKT |
| | | HTCPPCPAPELLGGPSVFLFPPKPKDTLMISRTPEVTCVVVDVSHEDPEVKF |
| | | NWYVDGVEVHNAKTKPCEEQYNSTYRCVSVLTVLHQDWLNGKEYKCKVSNKA |
| | | LPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHEA |
| | | LHNHYTQKSLSLSPGK |
| scFc-8 | 124 | DKTHTCPPCPAPELLGGPSVFLFPPKPKDTLMISRTPEVTCVVVDVSHEDPE |
| | | VKFNWYVDGVEVHNAKTKPCEEQYNSTYRCVSVLTVLHQDWLNGKEYK |
| | | CKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGN |
| | | VFSCSVMHEALHNHYTQKSLSLSPGGGGSGGGGSGGGGSGGGGSGGGGS |
| | | GGGGSDKTHTCPPCPAPELLGGPSVFLFPPKPKDTLMISRTPEVTCVVVDV |
| | | SHEDPEVKFNWYVDGVEVHNAKTKPCEEQYNSTYRCVSVLTVLHQDWL |
| | | NGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSR |
| | | WQQGNVFSCSVMHEALHNHYTQKSLSLSP |

The third domain of the bispecific antibody constructs used in the methods of the invention can be any of the scFc domains set forth in Table 5 or a variant of these scFc domains. In one embodiment, the bispecific antibody constructs according to the invention comprise a third domain comprising an amino acid sequence that is at least 90% identical to a sequence selected from SEQ ID NOs: 117-124. In another embodiment, the bispecific antibody constructs according to the invention comprise a third domain comprising an amino acid sequence selected from SEQ ID NOs: 117-124. In a preferred embodiment, the bispecific antibody constructs according to the invention comprise a third domain comprising the amino acid sequence of SEQ ID NO: 117. In another preferred embodiment, the bispecific antibody constructs according to the invention comprise a third domain comprising the amino acid sequence of SEQ ID NO: 118.

In certain embodiments, the bispecific antibody constructs used in the methods of the invention comprise, in an amino to carboxyl order:
(i) a first domain that specifically binds to human CD33 comprising a first immunoglobulin heavy chain variable region (VH1) and a first immunoglobulin light chain variable region (VL1);
(ii) a second domain that specifically binds to human CD3 comprising a second immunoglobulin heavy chain variable region (VH2) and a second immunoglobulin light chain variable region (VL2); and
(iii) a third domain comprising two Fc monomers.

In some embodiments, the bispecific antibody construct comprises, in amino to carboxyl order:

(i) a first domain that specifically binds to human CD33 comprising a VH1 comprising a CDRH1 having the sequence of SEQ ID NO: 10, a CDRH2 having a sequence selected from SEQ ID NOs: 11-13, and a CDRH3 having the sequence of SEQ ID NO: 14, and a VL1 comprising a CDRL1 having a sequence selected from SEQ ID NOs: 5-7, a CDRL2 having the sequence of SEQ ID NO: 8, and a CDRL3 having the sequence of SEQ ID NO: 9;

(ii) a second domain that specifically binds to human CD3 comprising a VH2 comprising a CDRH1 having a sequence selected from SEQ ID NOs: 37-42, a CDRH2 having a sequence selected from SEQ ID NOs: 43-47, and a CDRH3 having a sequence selected from SEQ ID NOs: 48-56, and a VL2 comprising a CDRL1 having a sequence selected from SEQ ID NOs: 30-32, a CDRL2 having the sequence of SEQ ID NO: 33 or SEQ ID NO: 34, and a CDRL3 having the sequence of SEQ ID NO: 35 or SEQ ID NO: 36; and

(iii) a third domain comprising two Fc monomers, each monomer comprising an immunoglobulin hinge region, a CH2 domain, and a CH3 domain, wherein said two monomers are fused to each other via a peptide linker. In such embodiments, VH1 comprises a sequence selected from SEQ ID NOs: 21-28 and VL1 comprises a sequence selected from SEQ ID NOs: 15-20. In these and other embodiments, VH2 comprises a sequence selected from SEQ ID NOs: 60-68 and VL2 comprises a sequence selected from SEQ ID NOs: 57-59. In one embodiment, VH1 comprises the sequence of SEQ ID NO: 28 and VL1 comprises the sequence of SEQ ID NO: 20. In a related embodiment, VH2 comprises the sequence of SEQ ID NO: 61 and VL2 comprises the sequence of SEQ ID NO: 59.

In a preferred embodiment, the bispecific antibody construct comprises, in amino to carboxyl order:

(i) a first domain that specifically binds to human CD33 comprising a VH1 comprising a CDRH1 having the sequence of SEQ ID NO: 10, a CDRH2 having the sequence of SEQ ID NO: 13, and a CDRH3 having the sequence of SEQ ID NO: 14, and a VL1 comprising a CDRL1 having the sequence of SEQ ID NO: 6, a CDRL2 having the sequence of SEQ ID NO: 8, and a CDRL3 having the sequence of SEQ ID NO: 9;

(ii) a second domain that specifically binds to human CD3 comprising a VH2 comprising a CDRH1 having the sequence of SEQ ID NO: 38, a CDRH2 having the sequence of SEQ ID NO: 44, and a CDRH3 having the sequence of SEQ ID NO: 49, and a VL2 comprising a CDRL1 having the sequence of SEQ ID NO: 32, a CDRL2 having the sequence of SEQ ID NO: 33, and a CDRL3 having the sequence of SEQ ID NO: 36; and

(iii) a third domain comprising two Fc monomers, each monomer comprising an immunoglobulin hinge region, a CH2 domain, and a CH3 domain, wherein said two monomers are fused to each other via a peptide linker.

In certain embodiments, peptide linkers, such as those described herein, connect the first domain to the second domain and/or the second domain to the third domain. Accordingly, in some embodiments, the bispecific antibody construct according to the invention comprises, in amino to carboxyl order:

(i) a first domain that specifically binds to human CD33;

(ii) a first peptide linker having an amino acid sequence selected from SEQ ID NOs: 70-72 and 80;

(iii) a second domain that specifically binds to human CD3;

(iv) a second peptide linker having an amino acid sequence selected from SEQ ID NOs: 69-72, and 78-80;

(v) a first Fc monomer;

(vi) a third peptide linker having an amino acid sequence selected SEQ ID NOs: 74-77; and

(vii) a second Fc monomer.

In certain embodiments, the bispecific antibody construct according to the invention comprises, in amino to carboxyl order:

(i) a first domain having an amino acid sequence selected from SEQ ID NOs: 83-91;

(ii) a first peptide linker having an amino acid sequence selected from SEQ ID NOs: 70-72 and 80;

(iii) a second domain having an amino acid sequence selected from SEQ ID NOs: 92-101;

(iv) a second peptide linker having an amino acid sequence selected from SEQ ID NOs: 69-72, and 78-80;

(v) a first Fc monomer having an amino acid sequence selected from SEQ ID NOs: 109-116;

(vi) a third peptide linker having an amino acid sequence selected SEQ ID NOs: 74-77; and

(vii) a second Fc monomer having an amino acid sequence selected from SEQ ID NOs: 109-116.

In a preferred embodiment, the bispecific antibody construct according to the invention comprises, in amino to carboxyl order:

(i) a first domain having the amino acid sequence of SEQ ID NO: 91;

(ii) a first peptide linker having the amino acid sequence of SEQ ID NO: 70 or SEQ ID NO: 80;

(iii) a second domain having the amino acid sequence of SEQ ID NO: 101;

(iv) a second peptide linker having the amino acid sequence of SEQ ID NO: 69 or SEQ ID NO: 70;

(v) a first Fc monomer having the amino acid sequence of SEQ ID NO: 109;

(vi) a third peptide linker having the amino acid sequence of SEQ ID NO: 75 or SEQ ID NO: 76; and

(vii) a second Fc monomer having the amino acid sequence of SEQ ID NO: 109.

In certain embodiments, the bispecific antibody constructs used in the methods of the invention are single chain antibody constructs. As used herein, a “single chain antibody construct” refers to an antibody construct consisting of only one polypeptide chain, i.e. all of the domains in the antibody construct are linked together, optionally via peptide linkers, to form a single polypeptide chain. One example of such a single chain antibody construct in the context of the present invention is a single chain polypeptide comprising, in an amino to carboxyl order, an anti-CD33 scFv domain, a first peptide linker, an anti-CD3 scFv domain, a second peptide linker, and an scFc domain. Exemplary anti-CD33×anti-CD3 bispecific single chain antibody constructs that can be used in the methods of the invention are described in WO 2017/134140, which is hereby incorporated by reference in its entirety, and are also set forth in Table 6 below.

TABLE 6
Exemplary Anti-CD33 x Anti-CD3 Bispecific Single Chain Antibody Constructs

| Designation | SEQ ID NO. | Amino Acid Sequence |
|---|---|---|
| scAb −1 | 125 | QVQLVQSGAEVKKPGESVKVSCKASGYTFTNYGMNWVKQAPGQCLEWMGWINTYTGEP |
| | | TYADKFQGRVTMTTDTSTSTAYMEIRNLGGDDTAVYYCARWSWSDGYYVYFDYWGQGT |
| | | SVTVSSGGGGSGGGGSGGGGSDIVMTQSPDSLTVSLGERTTINCKSSQSVLDSSTNKN |
| | | SLAWYQQKPGQPPKLLLSWASTRESGIPDRFSGSGSGTDFTLTIDSPQPEDSATYYCQ |
| | | QSAHFPITFGCGTRLEIKSGGGGSEVQLVESGGGLVQPGGSLKLSCAASGFTFNKYAM |
| | | NWVRQAPGKGLEWVARIRSKYNNYATYYADSVKDRFTISRDDSKNTAYLQMNNLKTED |
| | | TAVYYCVRHGNFGNSYISYWAYWGQGTLVTVSSGGGGSGGGGSGGGGSQTVVTQEPSL |
| | | TVSPGGTVTLTCGSSTGAVTSGNYPNWVQQKPGQAPRGLIGGTKFLAPGTPARFSGSL |
| | | LGGKAALTLSGVQPEDEAEYYCVLWYSNRWVFGGGTKLTVLGGGGDKTHTCPPCPAPE |
| | | LLGGPSVFLFPPKPKDTLMISRTPEVTCVVVDVSHEDPEVKFNWYVDGVEVHNAKTKP |
| | | CEEQYGSTYRCVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGKGGGGSGGGGSGGGGSGGG |
| | | GSGGGGSGGGGSDKTHTCPPCPAPELLGGPSVFLFPPKPKDTLMISRTPEVTCVVVDV |
| | | SHEDPEVKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSDIAVEWE |
| | | SNGQPENNYKTTPPVLDSDGSFFLYSKLTVDKSRWQQGNVFSCSVMHEALHNHYTQKS |
| | | LSLSPGK |
| scAb −2 | 126 | QVQLVQSGAEVKKPGESVKVSCKASGYTFTNYGMNWVKQAPGQCLEWMGWINTYTGEP |
| | | TYADKFQGRVTMTTDTSTSTAYMEIRNLGGDDTAVYYCARWSWSDGYYVYFDYWGQGT |
| | | SVTVSSGGGGSGGGGSGGGGSDIVMTQSPDSLTVSLGERTTINCKSSQSVLDSSTNKN |
| | | SLAWYQQKPGQPPKLLLSWASTRESGIPDRFSGSGSGTDFTLTIDSPQPEDSATYYCQ |
| | | QSAHFPITFGCGTRLEIKSGGGGSEVQLVESGGGLVQPGGSLKLSCAASGFTFNKYAM |
| | | NWVRQAPGKGLEWVARIRSKYNNYATYYADSVKDRFTISRDDSKNTAYLQMNNLKTED |
| | | TAVYYCVRHGNFGNSYISYWAYWGQGTLVTVSSGGGGSGGGGSGGGGSQTVVTQEPSL |
| | | TVSPGGTVTLTCGSSTGAVTSGNYPNWVQQKPGQAPRGLIGGTKFLAPGTPARFSGSL |
| | | LGGKAALTLSGVQPEDEAEYYCVLWYSNRWVFGGGTKLTVLGGGGDKTHTCPPCPAPE |
| | | LLGGPSVFLFPPKPKDTLMISRTPEVTCVVVDVSHEDPEVKFNWYVDGVEVHNAKTKP |
| | | CEEQYGSTYRCVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGGGGSGGGGSGGGGSGGGGS |
| | | GGGGSGGGGSDKTHTCPPCPAPELLGGPSVFLFPPKPKDTLMISRTPEVTCVVVDVSH |
| | | EDPEVKFNWYVDGVEVHNAKTKPCEEQYGSTYRCVSVLTVLHQDWLNGKEYKCKVSNK |
| | | ALPAPIEKTISKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEALHNHYTQKSLS |
| | | LSP |
| scAb −3 | 127 | QVQLVQSGAEVKKPGESVKVSCKASGYTFTNYGMNWVRQAPGQGLEWMGWINTYTGEP |
| | | TYADDFKGRVTMSSDTSTSTAYLEINSLRSDDTAIYYCARWSWSDGYYVYFDYWGQGT |
| | | TVTVSSGGGGSGGGGSGGGGSDIVMTQSPDSLTVSLGERTTINCKSSQSVLDSSKNKN |
| | | SLAWYQQKPGQPPKLLLSWASTRESGIPDRFSGSGSGTDFTLTIDSLQPEDSATYYCQ |
| | | QSAHFPITFGQGTRLEIKSGGGGSEVQLVESGGGLVQPGGSLKLSCAASGFTFNKYAM |
| | | NWVRQAPGKGLEWVARIRSKYNNYATYYADSVKDRFTISRDDSKNTAYLQMNNLKTED |
| | | TAVYYCVRHGNFGNSYISYWAYWGQGTLVTVSSGGGGSGGGGSGGGGSQTVVTQEPSL |
| | | TVSPGGTVTLTCGSSTGAVTSGNYPNWVQQKPGQAPRGLIGGTKFLAPGTPARFSGSL |
| | | LGGKAALTLSGVQPEDEAEYYCVLWYSNRWVFGGGTKLTVLGGGGDKTHTCPPCPAPE |
| | | LLGGPSVFLFPPKPKDTLMISRTPEVTCVVVDVSHEDPEVKFNWYVDGVEVHNAKTKP |
| | | CEEQYGSTYRCVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGKGGGGSGGGGSGGGGSGGG |
| | | GSGGGGSGGGGSDKTHTCPPCPAPELLGGPSVFLFPPKPKDTLMISRTPEVTCVVVDV |
| | | SHEDPEVKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSDIAVEWE |
| | | SNGQPENNYKTTPPVLDSDGSFFLYSKLTVDKSRWQQGNVFSCSVMHEALHNHYTQKS |
| | | LSLSPGK |
| scAb −4 | 128 | QVQLVQSGAEVKKPGASVKVSCKASGYTFTNYGMNWVKQAPGQGLKWMGWINTYTGEP |
| | | TYADDFKGRVTMTSDTSTSTAYLELHNLRSDDTAVYYCARWSWSDGYYVYFDYWGQGT |
| | | TVTVSSGGGGSGGGGSGGGGSDIVMTQSPDSLTVSLGERTTINCKSSQSVLDSSKNKN |
| | | SLAWYQQKPGQPPKLLLSWASTRESGIPDRFSGSGSGTDFTLTIDSLQPEDSATYYCQ |
| | | QSAHFPITFGQGTRLEIKSGGGGSEVQLVESGGGLVQPGGSLKLSCAASGFTFNKYAM |
| | | NWVRQAPGKGLEWVARIRSKYNNYATYYADSVKDRFTISRDDSKNTAYLQMNNLKTED |
| | | TAVYYCVRHGNFGNSYISYWAYWGQGTLVTVSSGGGGSGGGGSGGGGSQTVVTQEPSL |
| | | TVSPGGTVTLTCGSSTGAVTSGNYPNWVQQKPGQAPRGLIGGTKFLAPGTPARFSGSL |
| | | LGGKAALTLSGVQPEDEAEYYCVLWYSNRWVFGGGTKLTVLGGGGDKTHTCPPCPAPE |
| | | LLGGPSVFLFPPKPKDTLMISRTPEVTCVVVDVSHEDPEVKFNWYVDGVEVHNAKTKP |
| | | CEEQYGSTYRCVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGKGGGGSGGGGSGGGGSGGG |
| | | GSGGGGSGGGGSDKTHTCPPCPAPELLGGPSVFLFPPKPKDTLMISRTPEVTCVVVDV |
| | | SHEDPEVKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSDIAVEWE |
| | | SNGQPENNYKTTPPVLDSDGSFFLYSKLTVDKSRWQQGNVFSCSVMHEALHNHYTQKS |
| | | LSLSPGK |
| scAb −5 | 129 | QVQLVQSGAEVKKPGESVKVSCKASGYTFTNYGMNWVKQAPGQGLKWMGWINTYTGEP |
| | | TYADDFKGRVTMTTDTSTSTAYMEIRNLRNDDTAVYYCARWSWSDGYYVYFDYWGQGT |
| | | TVTVSSGGGGSGGGGSGGGGSDIVMTQSPDSLTVSLGERTTINCKSSQSVLDSSKNKN |
| | | SLAWYQQKPGQPPKLLLSWASTRESGIPDRFSGSGSGTDFTLTIDSLQPEDSATYYCQ |
| | | QSAHFPITFGQGTRLEIKSGGGGSEVQLVESGGGLVQPGGSLKLSCAASGFTFNKYAM |
| | | NWVRQAPGKGLEWVARIRSKYNNYATYYADSVKDRFTISRDDSKNTAYLQMNNLKTED |
| | | TAVYYCVRHGNFGNSYISYWAYWGQGTLVTVSSGGGGSGGGGSGGGGSQTVVTQEPSL |
| | | TVSPGGTVTLTCGSSTGAVTSGNYPNWVQQKPGQAPRGLIGGTKFLAPGTPARFSGSL |
| | | LGGKAALTLSGVQPEDEAEYYCVLWYSNRWVFGGGTKLTVLGGGGDKTHTCPPCPAPE |
| | | LLGGPSVFLFPPKPKDTLMISRTPEVTCVVVDVSHEDPEVKFNWYVDGVEVHNAKTKP |
| | | CEEQYGSTYRCVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGKGGGGSGGGGSGGGGSGGG |
| | | GSGGGGSGGGGSDKTHTCPPCPAPELLGGPSVFLFPPKPKDTLMISRTPEVTCVVVDV |
| | | SHEDPEVKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSDIAVEWE |
| | | SNGQPENNYKTTPPVLDSDGSFFLYSKLTVDKSRWQQGNVFSCSVMHEALHNHYTQKS |
| | | LSLSPGK |
| scAb −6 | 130 | QVQLVQSGAEVKKPGESVKVSCKASGYTFTNYGMNWVKQAPGQGLKWMGWINTYTGEP |
| | | TYADDFKGRVTMTSDTSTSTAYMEISSLRSDDTAVYYCARWSWSDGYYVYFDYWGQGT |
| | | TVTVSSGGGGSGGGGSGGGGSDIVMTQSPDSLTVSLGERTTINCKSSQSVLDSSKNKN |
| | | SLAWYQQKPGQPPKLLLSWASTRESGIPDRFSGSGSGTDFTLTIDSLQPEDSATYYCQ |
| | | QSAHFPITFGQGTRLEIKSGGGGSEVQLVESGGGLVQPGGSLKLSCAASGFTFNKYAM |
| | | NWVRQAPGKGLEWVARIRSKYNNYATYYADSVKDRFTISRDDSKNTAYLQMNNLKTED |
| | | TAVYYCVRHGNFGNSYISYWAYWGQGTLVTVSSGGGGSGGGGSGGGGSQTVVTQEPSL |
| | | TVSPGGTVTLTCGSSTGAVTSGNYPNWVQQKPGQAPRGLIGGTKFLAPGTPARFSGSL |
| | | LGGKAALTLSGVQPEDEAEYYCVLWYSNRWVFGGGTKLTVLGGGGDKTHTCPPCPAPE |
| | | LLGGPSVFLFPPKPKDTLMISRTPEVTCVVVDVSHEDPEVKFNWYVDGVEVHNAKTKP |
| | | CEEQYGSTYRCVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGKGGGGSGGGGSGGGGSGGG |
| | | GSGGGGSGGGGSDKTHTCPPCPAPELLGGPSVFLFPPKPKDTLMISRTPEVTCVVVDV |
| | | SHEDPEVKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSDIAVEWE |
| | | SNGQPENNYKTTPPVLDSDGSFFLYSKLTVDKSRWQQGNVFSCSVMHEALHNHYTQKS |
| | | LSLSPGK |
| scAb −7 | 131 | QVQLVQSGAEVKKPGESVKVSCKASGYTFTNYGMNWVKQAPGQGLEWMGWINTYTGET |
| | | NYADKFQGRVTFTSDTSTSTAYMELRNLKSDDTAVYYCARWSWSDGYYVYFDYWGQGT |
| | | TVTVSSGGGGSGGGGSGGGGSDIVMTQSPDSMTVSLGERTTINCKSSQSVLDSSTNKN |
| | | SLAWYQQKPGQPPKLLLSWASTRESGIPDRFSGSGSGTDFTLTIDSLQPEDSATYYCQ |
| | | QSAHFPITFGQGTRLDIKSGGGGSEVQLVESGGGLVQPGGSLKLSCAASGFTFNKYAM |
| | | NWVRQAPGKGLEWVARIRSKYNNYATYYADSVKDRFTISRDDSKNTAYLQMNNLKTED |
| | | TAVYYCVRHGNFGNSYISYWAYWGQGTLVTVSSGGGGSGGGGSGGGGSQTVVTQEPSL |
| | | TVSPGGTVTLTCGSSTGAVTSGNYPNWVQQKPGQAPRGLIGGTKFLAPGTPARFSGSL |
| | | LGGKAALTLSGVQPEDEAEYYCVLWYSNRWVFGGGTKLTVLGGGGDKTHTCPPCPAPE |
| | | LLGGPSVFLFPPKPKDTLMISRTPEVTCVVVDVSHEDPEVKFNWYVDGVEVHNAKTKP |
| | | CEEQYGSTYRCVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGKGGGGSGGGGSGGGGSGGG |
| | | GSGGGGSGGGGSDKTHTCPPCPAPELLGGPSVFLFPPKPKDTLMISRTPEVTCVVVDV |
| | | SHEDPEVKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSDIAVEWE |
| | | SNGQPENNYKTTPPVLDSDGSFFLYSKLTVDKSRWQQGNVFSCSVMHEALHNHYTQKS |
| | | LSLSPGK |
| scAb −8 | 132 | QVQLVQSGAEVKKPGESVKVSCKASGYTFTNYGMNWVKQAPGQGLEWMGWINTYTGET |
| | | NYADKFQGRVTFTSDTSTSTAYMELRNLKSDDTAVYYCARWSWSDGYYVYFDYWGQGT |
| | | TVTVSSGGGGSGGGGSGGGGSDIVMTQSPDSLSVSLGERTTINCKSSQSVLDSSTNKN |
| | | SLAWYQQKPGQPPKLLLSWASTRESGIPDRFSGSGSGTDFTLTIDSLQPEDSATYYCQ |
| | | QSAHFPITFGQGTRLEIKSGGGGSEVQLVESGGGLVQPGGSLKLSCAASGFTFNKYAM |
| | | NWVRQAPGKGLEWVARIRSKYNNYATYYADSVKDRFTISRDDSKNTAYLQMNNLKTED |
| | | TAVYYCVRHGNFGNSYISYWAYWGQGTLVTVSSGGGGSGGGGSGGGGSQTVVTQEPSL |
| | | TVSPGGTVTLTCGSSTGAVTSGNYPNWVQQKPGQAPRGLIGGTKFLAPGTPARFSGSL |
| | | LGGKAALTLSGVQPEDEAEYYCVLWYSNRWVFGGGTKLTVLGGGGDKTHTCPPCPAPE |
| | | LLGGPSVFLFPPKPKDTLMISRTPEVTCVVVDVSHEDPEVKFNWYVDGVEVHNAKTKP |
| | | CEEQYGSTYRCVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGKGGGGSGGGGSGGGGSGGG |
| | | GSGGGGSGGGGSDKTHTCPPCPAPELLGGPSVFLFPPKPKDTLMISRTPEVTCVVVDV |
| | | SHEDPEVKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSDIAVEWE |
| | | SNGQPENNYKTTPPVLDSDGSFFLYSKLTVDKSRWQQGNVFSCSVMHEALHNHYTQKS |
| | | LSLSPGK |
| scAb −9 | 133 | QVQLVQSGAEVKKPGESVKVSCKASGYTFTNYGMNWVKQAPGQGLEWMGWINTYTGEP |
| | | TYADKFQGRVTMTTDTSTSTAYMEIRNLRSDDTAVYYCARWSWSDGYYVYFDYWGQGT |
| | | TVTVSSGGGGSGGGGSGGGGSDIVMTQSPDSLTVSLGERTTINCKSSQSVLDSSNNKN |
| | | SLAWYQQKPGQPPKLLLSWASTRESGIPDRFSGSGSGTDFTLTIDGLQPEDSATYYCQ |
| | | QSAHFPITFGQGTRLEIKSGGGGSEVQLVESGGGLVQPGGSLKLSCAASGFTFNKYAM |
| | | NWVRQAPGKGLEWVARIRSKYNNYATYYADSVKDRFTISRDDSKNTAYLQMNNLKTED |
| | | TAVYYCVRHGNFGNSYISYWAYWGQGTLVTVSSGGGGSGGGGSGGGGSQTVVTQEPSL |
| | | TVSPGGTVTLTCGSSTGAVTSGNYPNWVQQKPGQAPRGLIGGTKFLAPGTPARFSGSL |
| | | LGGKAALTLSGVQPEDEAEYYCVLWYSNRWVFGGGTKLTVLGGGGDKTHTCPPCPAPE |
| | | LLGGPSVFLFPPKPKDTLMISRTPEVTCVVVDVSHEDPEVKFNWYVDGVEVHNAKTKP |
| | | CEEQYGSTYRCVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGKGGGGSGGGGSGGGGSGGG |
| | | GSGGGGSGGGGSDKTHTCPPCPAPELLGGPSVFLFPPKPKDTLMISRTPEVTCVVVDV |
| | | SHEDPEVKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSDIAVEWE |
| | | SNGQPENNYKTTPPVLDSDGSFFLYSKLTVDKSRWQQGNVFSCSVMHEALHNHYTQKS |
| | | LSLSPGK |
| scAb −10 | 134 | QVQLVQSGAEVKKPGESVKVSCKASGYTFTNYGMNWVKQAPGQGLEWMGWINTYTGEP |
| | | TYADKFQGRVTMTTDTSTSTAYMEIRNLGGDDTAVYYCARWSWSDGYYVYFDYWGQGT |
| | | SVTVSSGGGGSGGGGSGGGGSDIVMTQSPDSLTVSLGERTTINCKSSQSVLDSSTNKN |
| | | SLAWYQQKPGQPPKLLLSWASTRESGIPDRFSGSGSGTDFTLTIDSPQPEDSATYYCQ |
| | | QSAHFPITFGQGTRLEIKSGGGGSEVQLVESGGGLVQPGGSLKLSCAASGFTFNKYAM |
| | | NWVRQAPGKGLEWVARIRSKYNNYATYYADSVKDRFTISRDDSKNTAYLQMNNLKTED |
| | | TAVYYCVRHGNFGNSYISYWAYWGQGTLVTVSSGGGGSGGGGSGGGGSQTVVTQEPSL |
| | | TVSPGGTVTLTCGSSTGAVTSGNYPNWVQQKPGQAPRGLIGGTKFLAPGTPARFSGSL |
| | | LGGKAALTLSGVQPEDEAEYYCVLWYSNRWVFGGGTKLTVLGGGGDKTHTCPPCPAPE |
| | | LLGGPSVFLFPPKPKDTLMISRTPEVTCVVVDVSHEDPEVKFNWYVDGVEVHNAKTKP |
| | | CEEQYGSTYRCVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGKGGGGSGGGGSGGGGSGGG |
| | | GSGGGGSGGGGSDKTHTCPPCPAPELLGGPSVFLFPPKPKDTLMISRTPEVTCVVVDV |
| | | SHEDPEVKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSDIAVEWE |
| | | SNGQPENNYKTTPPVLDSDGSFFLYSKLTVDKSRWQQGNVFSCSVMHEALHNHYTQKS |
| | | LSLSPGK |
| scAb −11 | 135 | QVQLVQSGAEVKKPGESVKVSCKASGYTFTNYGMNWVKQAPGQGLEWMGWINTYTGEP |
| | | TYADKFQGRVTMTTDTSTSTAYMEIRNLGGDDTAVYYCARWSWSDGYYVYFDYWGQGT |
| | | SVTVSSGGGGSGGGGSGGGGSDIVMTQSPDSLTVSLGERTTINCKSSQSVLDSSTNKN |
| | | SLAWYQQKPGQPPKLLLSWASTRESGIPDRFSGSGSGTDFTLTIDSPQPEDSATYYCQ |
| | | QSAHFPITFGQGTRLEIKSGGGGSEVQLVESGGGLVQPGGSLKLSCAASGFTFNKYAM |
| | | NWVRQAPGKGLEWVARIRSKYNNYATYYADSVKDRFTISRDDSKNTAYLQMNNLKTED |
| | | TAVYYCVRHGNFGNSYISYWAYWGQGTLVTVSSGGGGSGGGGSGGGGSQTVVTQEPSL |
| | | TVSPGGTVTLTCGSSTGAVTSGNYPNWVQQKPGQAPRGLIGGTKFLAPGTPARFSGSL |
| | | LGGKAALTLSGVQPEDEAEYYCVLWYSNRWVFGGGTKLTVLGGGGDKTHTCPPCPAPE |
| | | LLGGPSVFLFPPKPKDTLMISRTPEVTCVVVDVSHEDPEVKFNWYVDGVEVHNAKTKP |
| | | CEEQYGSTYRCVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGGGGSGGGGSGGGGSGGGGS |
| | | GGGGSGGGGSDKTHTCPPCPAPELLGGPSVFLFPPKPKDTLMISRTPEVTCVVVDVSH |
| | | EDPEVKFNWYVDGVEVHNAKTKPCEEQYGSTYRCVSVLTVLHQDWLNGKEYKCKVSNK |
| | | ALPAPIEKTISKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEALHNHYTQKSLS |
| | | LSP |
| scAb −12 | 136 | QVQLVQSGAEVKKPGESVKVSCKASGYTFTNYGMNWVKQAPGQCLEWMGWINTYTGEP |
| | | TYADKFQGRVTMTTDTSTSTAYMEIRNLGGDDTAVYYCARWSWSDGYYVYFDYWGQGT |
| | | SVTVSSGGGGSGGGGSGGGGSDIVMTQSPDSLTVSLGERTTINCKSSQSVLDSSTNKN |
| | | SLAWYQQKPGQPPKLLLSWASTRESGIPDRFSGSGSGTDFTLTIDSPQPEDSATYYCQ |
| | | QSAHFPITFGCGTRLEIKSGGGGSEVQLVESGGGLVQPGGSLKLSCAASGFTFNKYAM |
| | | NWVRQAPGKGLEWVARIRSKYNNYATYYADSVKDRFTISRDDSKNTAYLQMNNLKTED |
| | | TAVYYCVRHGNFGNSYISYWAYWGQGTLVTVSSGGGGSGGGGSGGGGSQTVVTQEPSL |
| | | TVSPGGTVTLTCGSSTGAVTSGYYPNWVQQKPGQAPRGLIGGTKFLAPGTPARFSGSL |
| | | LGGKAALTLSGVQPEDEAEYYCALWYSNRWVFGGGTKLTVLGGGGDKTHTCPPCPAPE |
| | | LLGGPSVFLFPPKPKDTLMISRTPEVTCVVVDVSHEDPEVKFNWYVDGVEVHNAKTKP |
| | | CEEQYGSTYRCVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGKGGGGSGGGGSGGGGSGGG |
| | | GSGGGGSGGGGSDKTHTCPPCPAPELLGGPSVFLFPPKPKDTLMISRTPEVTCVVVDV |
| | | SHEDPEVKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSDIAVEWE |
| | | SNGQPENNYKTTPPVLDSDGSFFLYSKLTVDKSRWQQGNVFSCSVMHEALHNHYTQKS |
| | | LSLSPGK |
| scAb −13 | 137 | QVQLVQSGAEVKKPGESVKVSCKASGYTFTNYGMNWVKQAPGQCLEWMGWINTYTGEP |
| | | TYADKFQGRVTMTTDTSTSTAYMEIRNLGGDDTAVYYCARWSWSDGYYVYFDYWGQGT |
| | | SVTVSSGGGGSGGGGSGGGGSDIVMTQSPDSLTVSLGERTTINCKSSQSVLDSSTNKN |
| | | SLAWYQQKPGQPPKLLLSWASTRESGIPDRFSGSGSGTDFTLTIDSPQPEDSATYYCQ |
| | | QSAHFPITFGCGTRLEIKSGGGGSEVQLVESGGGLVQPGGSLKLSCAASGFTFNSYAM |
| | | NWVRQAPGKGLEWVARIRSKYNNYATYYADSVKGRFTISRDDSKNTAYLQMNNLKTED |
| | | TAVYYCVRHGNFGNSYVSWWAYWGQGTLVTVSSGGGGSGGGGSGGGGSQTVVTQEPSL |
| | | TVSPGGTVTLTCGSSTGAVTSGNYPNWVQQKPGQAPRGLIGGTKFLAPGTPARFSGSL |
| | | LGGKAALTLSGVQPEDEAEYYCVLWYSNRWVFGGGTKLTVLGGGGDKTHTCPPCPAPE |
| | | LLGGPSVFLFPPKPKDTLMISRTPEVTCVVVDVSHEDPEVKFNWYVDGVEVHNAKTKP |
| | | CEEQYGSTYRCVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGKGGGGSGGGGSGGGGSGGG |
| | | GSGGGGSGGGGSDKTHTCPPCPAPELLGGPSVFLFPPKPKDTLMISRTPEVTCVVVDV |
| | | SHEDPEVKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSDIAVEWE |
| | | SNGQPENNYKTTPPVLDSDGSFFLYSKLTVDKSRWQQGNVFSCSVMHEALHNHYTQKS |
| | | LSLSPGK |
| scAb −14 | 138 | QVQLVQSGAEVKKPGESVKVSCKASGYTFTNYGMNWVKQAPGQGLEWMGWINTYTGEP |
| | | TYADKFQGRVTMTTDTSTSTAYMEIRNLGGDDTAVYYCARWSWSDGYYVYFDYWGQGT |
| | | SVTVSSGGGGSGGGGSGGGGSDIVMTQSPDSLTVSLGERTTINCKSSQSVLDSSTNKN |
| | | SLAWYQQKPGQPPKLLLSWASTRESGIPDRFSGSGSGTDFTLTIDSPQPEDSATYYCQ |
| | | QSAHFPITFGQGTRLEIKSGGGGSEVQLVESGGGLVQPGGSLKLSCAASGFTFNKYAM |
| | | NWVRQAPGKGLEWVARIRSKYNNYATYYADSVKDRFTISRDDSKNTAYLQMNNLKTED |
| | | TAVYYCVRHGNFGNSYISYWAYWGQGTLVTVSSGGGGSGGGGSGGGGSQTVVTQEPSL |
| | | TVSPGGTVTLTCGSSTGAVTSGYYPNWVQQKPGQAPRGLIGGTKFLAPGTPARFSGSL |
| | | LGGKAALTLSGVQPEDEAEYYCALWYSNRWVFGGGTKLTVLGGGGDKTHTCPPCPAPE |
| | | LLGGPSVFLFPPKPKDTLMISRTPEVTCVVVDVSHEDPEVKFNWYVDGVEVHNAKTKP |
| | | CEEQYGSTYRCVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGKGGGGSGGGGSGGGGSGGG |
| | | GSGGGGSGGGGSDKTHTCPPCPAPELLGGPSVFLFPPKPKDTLMISRTPEVTCVVVDV |
| | | SHEDPEVKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSDIAVEWE |
| | | SNGQPENNYKTTPPVLDSDGSFFLYSKLTVDKSRWQQGNVFSCSVMHEALHNHYTQKS |
| | | LSLSPGK |
| scAb −15 | 139 | QVQLVQSGAEVKKPGESVKVSCKASGYTFTNYGMNWVKQAPGQGLEWMGWINTYTGEP |
| | | TYADKFQGRVTMTTDTSTSTAYMEIRNLGGDDTAVYYCARWSWSDGYYVYFDYWGQGT |
| | | SVTVSSGGGGSGGGGSGGGGSDIVMTQSPDSLTVSLGERTTINCKSSQSVLDSSTNKN |
| | | SLAWYQQKPGQPPKLLLSWASTRESGIPDRFSGSGSGTDFTLTIDSPQPEDSATYYCQ |
| | | QSAHFPITFGQGTRLEIKSGGGGSEVQLVESGGGLVQPGGSLKLSCAASGFTFNSYAM |
| | | NWVRQAPGKGLEWVARIRSKYNNYATYYADSVKGRFTISRDDSKNTAYLQMNNLKTED |
| | | TAVYYCVRHGNFGNSYVSWWAYWGQGTLVTVSSGGGGSGGGGSGGGGSQTVVTQEPSL |
| | | TVSPGGTVTLTCGSSTGAVTSGNYPNWVQQKPGQAPRGLIGGTKFLAPGTPARFSGSL |
| | | LGGKAALTLSGVQPEDEAEYYCVLWYSNRWVFGGGTKLTVLGGGGDKTHTCPPCPAPE |
| | | LLGGPSVFLFPPKPKDTLMISRTPEVTCVVVDVSHEDPEVKFNWYVDGVEVHNAKTKP |
| | | CEEQYGSTYRCVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGKGGGGSGGGGSGGGGSGGG |
| | | GSGGGGSGGGGSDKTHTCPPCPAPELLGGPSVFLFPPKPKDTLMISRTPEVTCVVVDV |
| | | SHEDPEVKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSDIAVEWE |
| | | SNGQPENNYKTTPPVLDSDGSFFLYSKLTVDKSRWQQGNVFSCSVMHEALHNHYTQKS |
| | | LSLSPGK |

In certain embodiments, the bispecific antibody construct administered to a patient according to the methods of the invention comprises an amino acid sequence selected from SEQ ID NOs: 125-139. In one embodiment, the bispecific antibody construct comprises the amino acid sequence of SEQ ID NO: 126. In another embodiment, the bispecific antibody construct comprises the amino acid sequence of SEQ ID NO: 134. In yet another embodiment, the bispecific antibody construct comprises the amino acid sequence of SEQ ID NO: 135. In a preferred embodiment, the bispecific antibody construct used in the methods of the invention comprises the amino acid sequence of SEQ ID NO: 125.

The anti-CD33×anti-CD3 bispecific antibody constructs employed in the methods of the invention may be variants of the single chain antibody constructs shown in Table 6 and comprise an amino acid sequence that is at least 90%, 91%, 92%, 93%, 94%, 95%, 96%, 97%, 98% or 99% identical to an amino acid sequence of SEQ ID NOs: 125-139. In one embodiment, the bispecific antibody construct comprises an amino acid sequence that is at least 95% identical to an amino acid sequence selected from SEQ ID NOs: 125-139. In another embodiment, the bispecific antibody construct comprises an amino acid sequence that is at least 98% identical to an amino acid sequence selected from SEQ ID NOs: 125-139. In certain embodiments, the sequence variability occurs in the peptide linker regions and/or the single-chain Fc domain.

The anti-CD33×anti-CD3 bispecific antibody constructs for use in the methods of the invention may be prepared by any of a number of conventional techniques. For example, the anti-CD33×anti-CD3 bispecific antibody constructs described herein may be produced by recombinant expression systems, using any technique known in the art. See, e.g., Monoclonal Antibodies, Hybridomas: A New Dimension in Biological Analyses, Kennet et al. (eds.) Plenum Press, New York (1980); and Antibodies: A Laboratory Manual, Harlow and Lane (eds.), Cold Spring Harbor Laboratory Press, Cold Spring Harbor, N.Y. (1988).

Anti-CD33×anti-CD3 bispecific antibody constructs or components thereof (e.g. Fv fragments, Fc monomers) can be expressed in hybridoma cell lines or in cell lines other than hybridomas. Expression constructs encoding the bispecific antibody constructs can be used to transform a mammalian, insect or microbial host cell. Transformation can be performed using any known method for introducing polynucleotides into a host cell, including, for example packaging the polynucleotide in a virus or bacteriophage and transducing a host cell with the construct by transfection procedures known in the art, as exemplified by U.S. Pat. Nos. 4,399,216; 4,912,040; 4,740,461; 4,959,455. The optimal transformation procedure used will depend upon which type of host cell is being transformed. Methods for introduction of heterologous polynucleotides into mammalian cells are well known in the art and include, but are not limited to, dextran-mediated transfection, calcium phosphate precipitation, polybrene mediated transfection, protoplast fusion, electroporation, encapsulation of the polynucleotide(s) in liposomes, mixing nucleic acid with positively-charged lipids, and direct microinjection of the DNA into nuclei.

Recombinant expression constructs typically comprise a nucleic acid molecule encoding a polypeptide comprising one or more of the following: one or more CDRs provided herein; a light chain constant region; a light chain variable region; a heavy chain constant region (e.g., CH1, CH2 and/or CH3); a heavy chain variable region; hinge region, Fc region, and/or another scaffold portion of an anti-CD33 antibody or anti-CD3 antibody. These nucleic acid sequences are inserted into an appropriate expression vector using standard ligation techniques. In embodiments in which the bispecific antibody construct is a single chain antibody construct, the nucleic acid comprised in the recombinant expression vector will typically encode the full-length antibody construct (e.g. full-length fusion protein). The vector is typically selected to be functional in the particular host cell employed (i.e., the vector is compatible with the host cell machinery, permitting amplification and/or expression of the gene can occur). In some embodiments, vectors are used that employ protein-fragment complementation assays using protein reporters, such as dihydrofolate reductase (see, for example, U.S. Pat. No. 6,270,964, which is hereby incorporated by reference). Suitable expression vectors can be purchased, for example, from Invitrogen Life Technologies or BD Biosciences (formerly “Clontech”). Other useful vectors for cloning and expressing the antibody constructs and fragments include those described in Bianchi and McGrew, 2003, Biotech. Biotechnol. Bioeng. 84:439-44, which is hereby incorporated by reference. Additional suitable expression vectors are discussed, for example, in Methods Enzymol., vol. 185 (D. V. Goeddel, ed.), 1990, New York: Academic Press.

Typically, expression vectors used in any of the host cells will contain sequences for plasmid maintenance and for cloning and expression of exogenous nucleotide sequences. Such sequences, collectively referred to as “flanking sequences” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Each of these sequences is discussed below.

Optionally, the vector may contain a “tag”-encoding sequence, i.e., an oligonucleotide molecule located at the 5′ or 3′ end of the anti-CD33×anti-CD3 bispecific antibody construct coding sequence; the oligonucleotide sequence encodes polyHis (such as hexaHis), or another “tag” such as FLAG®, HA (hemaglutinin influenza virus), or myc, for which commercially available antibodies exist. This tag is typically fused to the polypeptide upon expression of the polypeptide, and can serve as a means for affinity purification or detection of the anti-CD33×anti-CD3 bispecific antibody construct from the host cell. Affinity purification can be accomplished, for example, by column chromatography using antibodies against the tag as an affinity matrix. Optionally, the tag can subsequently be removed from the purified antibody construct by various means such as using certain peptidases for cleavage.

Flanking sequences may be homologous (i.e., from the same species and/or strain as the host cell), heterologous (i.e., from a species other than the host cell species or strain), hybrid (i.e., a combination of flanking sequences from more than one source), synthetic or native. As such, the source of a flanking sequence may be any prokaryotic or eukaryotic organism, any vertebrate or invertebrate organism, or any plant, provided that the flanking sequence is functional in, and can be activated by, the host cell machinery.

Flanking sequences useful in the vectors may be obtained by any of several methods well known in the art. Typically, flanking sequences useful herein will have been previously identified by mapping and/or by restriction endonuclease digestion and can thus be isolated from the proper tissue source using the appropriate restriction endonucleases. In some cases, the full nucleotide sequence of a flanking sequence may be known. Here, the flanking sequence may be synthesized using the methods described herein for nucleic acid synthesis or cloning.

Whether all or only a portion of the flanking sequence is known, it may be obtained using polymerase chain reaction (PCR) and/or by screening a genomic library with a suitable probe such as an oligonucleotide and/or flanking sequence fragment from the same or another species. Where the flanking sequence is not known, a fragment of DNA containing a flanking sequence may be isolated from a larger piece of DNA that may contain, for example, a coding sequence or even another gene or genes. Isolation may be accomplished by restriction endonuclease digestion to produce the proper DNA fragment followed by isolation using agarose gel purification, Qiagen® column chromatography (Chatsworth, Calif.), or other methods known to the skilled artisan. The selection of suitable enzymes to accomplish this purpose will be readily apparent to one of ordinary skill in the art.

An origin of replication is typically a part of those prokaryotic expression vectors purchased commercially, and the origin aids in the amplification of the vector in a host cell. If the vector of choice does not contain an origin of replication site, one may be chemically synthesized based on a known sequence, and ligated into the vector. For example, the origin of replication from the plasmid pBR322 (New England Biolabs, Beverly, Mass.) is suitable for most gram-negative bacteria, and various viral origins (e.g., SV40, polyoma, adenovirus, vesicular stomatitus virus (VSV), or papillomaviruses such as HPV or BPV) are useful for cloning vectors in mammalian cells. Generally, the origin of replication component is not needed for mammalian expression vectors (for example, the SV40 origin is often used only because it also contains the virus early promoter).

A transcription termination sequence is typically located 3′ to the end of a polypeptide coding region and serves to terminate transcription. Usually, a transcription termination sequence in prokaryotic cells is a G-C rich fragment followed by a poly-T sequence. While the sequence is easily cloned from a library or even purchased commercially as part of a vector, it can also be readily synthesized using known methods for nucleic acid synthesis.

A selectable marker gene encodes a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells; (b) complement auxotrophic deficiencies of the cell; or (c) supply critical nutrients not available from complex or defined media. Specific selectable markers are the kanamycin resistance gene, the ampicillin resistance gene, and the tetracycline resistance gene. Advantageously, a neomycin resistance gene may also be used for selection in both prokaryotic and eukaryotic host cells.

Other selectable genes may be used to amplify the gene that will be expressed. Amplification is the process wherein genes that are required for production of a protein critical for growth or cell survival are reiterated in tandem within the chromosomes of successive generations of recombinant cells. Examples of suitable selectable markers for mammalian cells include dihydrofolate reductase (DHFR) and promoterless thymidine kinase genes. Mammalian cell transformants are placed under selection pressure wherein only the transformants are uniquely adapted to survive by virtue of the selectable gene present in the vector. Selection pressure is imposed by culturing the transformed cells under conditions in which the concentration of selection agent in the medium is successively increased, thereby leading to the amplification of both the selectable gene and the DNA that encodes another gene, such as an anti-CD33×anti-CD3 bispecific antibody construct. As a result, increased quantities of a polypeptide, such as an anti-CD33×anti-CD3 bispecific antibody construct, are synthesized from the amplified DNA.

A ribosome-binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of the polypeptide to be expressed.

In some cases, such as where glycosylation is desired in a eukaryotic host cell expression system, one may manipulate the various pre- or pro-sequences to improve glycosylation or yield. For example, one may alter the peptidase cleavage site of a particular signal peptide, or add prosequences, which also may affect glycosylation. The final protein product may have, in the −1 position (relative to the first amino acid of the mature protein), one or more additional amino acids incident to expression, which may not have been totally removed. For example, the final protein product may have one or two amino acid residues found in the peptidase cleavage site, attached to the amino terminus. Alternatively, use of some enzyme cleavage sites may result in a slightly truncated form of the desired polypeptide, if the enzyme cuts at such area within the mature polypeptide.

Expression and cloning vectors will typically contain a promoter that is recognized by the host organism and operably linked to the molecule encoding an anti-CD33×anti-CD3 bispecific antibody construct. Promoters are untranscribed sequences located upstream (i.e., 5′) to the start codon of a structural gene (generally within about 100 to 1000 bp) that control transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, uniformly transcribe a gene to which they are operably linked, that is, with little or no control over gene expression. A large number of promoters, recognized by a variety of potential host cells, are well known. A suitable promoter is operably linked to the nucleic acid encoding the anti-CD33×anti-CD3 bispecific antibody construct or component thereof by removing the promoter from the source DNA by restriction enzyme digestion and inserting the desired promoter sequence into the vector.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus, and Simian Virus 40 (SV40). Other suitable mammalian promoters include heterologous mammalian promoters, for example, heat-shock promoters and the actin promoter.

An enhancer sequence may be inserted into the vector to increase transcription of a nucleic acid encoding an anti-CD33×anti-CD3 bispecific antibody construct or component thereof by higher eukaryotes. Enhancers are cis-acting elements of DNA, usually about 10-300 bp in length, that act on the promoter to increase transcription. Enhancers are relatively orientation and position independent, having been found at positions both 5′ and 3′ to the transcription unit. Several enhancer sequences available from mammalian genes are known (e.g., globin, elastase, albumin, alpha-feto-protein and insulin). Typically, however, an enhancer from a virus is used. The SV40 enhancer, the cytomegalovirus early promoter enhancer, the polyoma enhancer, and adenovirus enhancers known in the art are exemplary enhancing elements for the activation of eukaryotic promoters. While an enhancer may be positioned in the vector either 5′ or 3′ to a coding sequence, it is typically located at a site 5′ from the promoter. A sequence encoding an appropriate native or heterologous signal sequence (leader sequence or signal peptide) can be incorporated into an expression vector, to promote extracellular secretion of the antibody construct. The choice of signal peptide or leader depends on the type of host cells in which the antibody construct is to be produced, and a heterologous signal sequence can replace the native signal sequence. Examples of signal peptides that are functional in mammalian host cells include the following: the signal sequence for interleukin 7 (IL-7) described in U.S. Pat. No. 4,965,195; the signal sequence for interleukin-2 receptor described in Cosman et al., 1984, Nature 312:768; the interleukin-4 receptor signal peptide described in EP Patent No. 0367 566; the type I interleukin-1 receptor signal peptide described in U.S. Pat. No. 4,968,607; the type II interleukin-1 receptor signal peptide described in EP Patent No. 0 460 846.

The expression vectors for recombinant production of the anti-CD33×anti-CD3 bispecific antibody constructs described herein may be constructed from a starting vector such as a commercially available vector. Such vectors may or may not contain all of the desired flanking sequences. Where one or more of the flanking sequences described herein are not already present in the vector, they may be individually obtained and ligated into the vector. Methods used for obtaining each of the flanking sequences are well known to one skilled in the art.

After the vector has been constructed and a nucleic acid molecule encoding the anti-CD33×anti-CD3 bispecific antibody construct or component thereof has been inserted into the proper site of the vector, the completed vector may be inserted into a suitable host cell for amplification and/or polypeptide expression. The transformation of an expression vector for a polypeptide into a selected host cell may be accomplished by well-known methods including transfection, infection, calcium phosphate co-precipitation, electroporation, microinjection, lipofection, DEAE-dextran mediated transfection, or other known techniques. The method selected will in part be a function of the type of host cell to be used. These methods and other suitable methods are well known to the skilled artisan, and are set forth, for example, in Sambrook et al., 2001, supra.

A host cell, when cultured under appropriate conditions, synthesizes an antibody construct that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

Exemplary host cells include prokaryote, yeast, or higher eukaryote cells. Prokaryotic host cells include eubacteria, such as Gram-negative or Gram-positive organisms, for example, Enterobacteriaceae such as Escherichia, e.g., E. coli, Enterobacter, Envinia, Klebsiella, Proteus, Salmonella, e.g., Salmonella typhimurium, Serratia, e.g., Serratia marcescans, and Shigella, as well as Bacillus, such as B. subtilis and B. licheniformis, Pseudomonas, and Streptomyces. Eukaryotic microbes such as filamentous fungi or yeast are suitable cloning or expression hosts for recombinant polypeptides. Saccharomyces cerevisiae, or common baker's yeast, is the most commonly used among lower eukaryotic host microorganisms. However, a number of other genera, species, and strains are commonly available and useful herein, such as Pichia, e.g. P. pastoris, Schizosaccharomyces pombe; Kluyveromyces, Yarrowia; Candida; Trichoderma reesia; Neurospora crassa; Schwanniomyces, such as Schwanniomyces occidentalis; and filamentous fungi, such as, e.g., Neurospora, Penicillium, Tolypocladium, and Aspergillus hosts such as A. nidulans and A. niger.

Host cells for the expression of glycosylated proteins can be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection of such cells are publicly available, e.g., the L-1 variant of Autographa californica NPV and the Bm-5 strain of Bombyx mori NPV.

Vertebrate host cells are also suitable hosts, and recombinant production of antibody constructs from such cells has become routine procedure. Mammalian cell lines available as hosts for expression are well known in the art and include, but are not limited to, immortalized cell lines available from the American Type Culture Collection (ATCC), including but not limited to Chinese hamster ovary (CHO) cells, including CHOK1 cells (ATCC CCL61), DXB-11, DG-44, and Chinese hamster ovary cells/-DHFR (CHO, Urlaub et al., Proc. Natl. Acad. Sci. USA 77: 4216, 1980); monkey kidney CV1 line transformed by SV40 (COS-7, ATCC CRL 1651); human embryonic kidney line (293 or 293 cells subcloned for growth in suspension culture, (Graham et al., J. Gen Virol. 36: 59, 1977); baby hamster kidney cells (BHK, ATCC CCL 10); mouse sertoli cells (TM4, Mather, Biol. Reprod. 23: 243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TM cells (Mather et al., Annals N.Y Acad. Sci. 383: 44-68, 1982); MRC 5 cells or FS4 cells; mammalian myeloma cells, and a number of other cell lines. CHO cells are preferred host cells in some embodiments for expressing the anti-CD33×anti-CD3 bispecific antibody constructs.

Host cells are transformed or transfected with the above-described expression vectors for production of the antibody constructs and are cultured in conventional nutrient media modified as appropriate for inducing promoters, selecting transformants, or amplifying the genes encoding the desired sequences. The host cells used to produce the antibody constructs may be cultured in a variety of media. Commercially available media such as Ham's F10 (Sigma), Minimal Essential Medium (MEM, Sigma), RPMI-1640 (Sigma), and Dulbecco's Modified Eagle's Medium (DMEM, Sigma) are suitable for culturing the host cells. In addition, any of the media described in Ham et al., Meth. Enz. 58: 44, 1979; Barnes et al., Anal. Biochem. 102: 255, 1980; U.S. Pat. Nos. 4,767,704; 4,657,866; 4,927,762; 4,560,655; or 5,122,469; WO 90/03430; or WO 87/00195 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression and will be apparent to the ordinary skilled artisan.

Upon culturing the host cells, the antibody construct can be produced intracellularly, in the periplasmic space, or directly secreted into the medium. If the antibody construct is produced intracellularly, as a first step, the host cells are lysed (e.g., by mechanical shear, osmotic shock, or enzymatic methods) and the particulate debris (e.g., host cells and lysed fragments), is removed, for example, by centrifugation, microfiltration, or ultrafiltration. If the antibody construct is secreted into the culture medium, the antibody construct can be separated from host cells through centrifugation or microfiltration, and optionally, subsequently concentrated through ultrafiltration. The anti-CD33×anti-CD3 bispecific antibody constructs can be further purified or partially purified using, for example, one or more chromatography steps, such as affinity chromatography (e.g. protein A or protein G affinity chromatography), cation exchange chromatography, anion exchange chromatography, hydroxyapatite chromatography, hydrophobic interaction chromatography, or mixed mode chromatography.

The anti-CD33×anti-CD3 bispecific antibody construct is generally administered to the patient in a pharmaceutical composition, which can include pharmaceutically-acceptable carriers, excipients, or diluents. “Pharmaceutically-acceptable” refers to molecules, compounds, and compositions that are non-toxic to human recipients at the dosages and concentrations employed and/or do not produce allergic or adverse reactions when administered to humans. In certain embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. Methods and suitable materials for formulating molecules for therapeutic use are known in the pharmaceutical arts, and are described, for example, in REMINGTON'S PHARMACEUTICAL SCIENCES, 18th Edition, (A. R. Genrmo, ed.), 1990, Mack Publishing Company. Pharmaceutical compositions comprising the bispecific antibody constructs to be administered according to the methods of the invention include, but are not limited to, liquid, frozen, and lyophilized compositions.

If the pharmaceutical composition has been lyophilized, the lyophilized material is reconstituted in an appropriate liquid prior to administration. The lyophilized material may be reconstituted in, e.g., bacteriostatic water for injection (BWFI), physiological saline, phosphate buffered saline (PBS), or the same formulation the protein had been in prior to lyophilization.

In some embodiments, the selection of carriers and excipients for incorporation into the pharmaceutical compositions influences the physical state, stability, rate of in vivo release and rate of in vivo clearance of the bispecific antibody constructs. In certain embodiments, the primary vehicle or carrier in a pharmaceutical composition may be either aqueous or non-aqueous in nature. For example, a suitable vehicle or carrier may be water for injection, physiological saline solution, possibly supplemented with other materials or excipients common in compositions for parenteral administration.

In certain embodiments of the methods described herein, the anti-CD33×anti-CD3 bispecific antibody construct (e.g. a pharmaceutical composition comprising the anti-CD33×anti-CD3 bispecific antibody construct) is administered to the patient parenterally. Parenteral administration refers to administration of the molecule by routes other than through the gastrointestinal tract and can include intraperitoneal, intramuscular, intravenous, intraarterial, intradermal, subcutaneous, intracerebral, intracerebroventricular, and intrathecal administration. In some embodiments, administration of the anti-CD33×anti-CD3 bispecific antibody construct according to the methods of the invention is intravenous.

Parenteral or intravenous administration can be performed by injection (e.g. using a needle and a syringe) or by infusion (e.g. via a catheter and a pump system). It is envisaged that the administration according to the present invention is via intravenous injection or via intravenous infusion. Usually, an intravenous (IV) infusion is administered via a line, a port or a catheter (small, flexible tube), such as a central venous access or a central venous catheter (CVC), which is a catheter placed into a large vein, or a peripheral venous catheter (PVC), which is a catheter placed into a peripheral vein. In general, catheters or lines can be placed in veins in the neck (internal jugular vein), chest (subclavian vein or axillary vein), groin (femoral vein), or through veins in the arms (also known as a PICC line, or peripherally inserted central catheters). Central IV lines have catheters that are advanced through a vein and empty into a large central vein, usually the superior vena cava, inferior vena cava or even the right atrium of the heart. A peripheral intravenous (PIV) line is used on peripheral veins (the veins in the arms, hands, legs and feet). A port is a central venous line that does not have an external connector; instead, it has a small reservoir that is covered with silicone rubber and is implanted under the skin. Medication is administered intermittently by placing a small needle through the skin, piercing the silicone, into the reservoir. When the needle is withdrawn, the reservoir cover reseals itself. The cover can accept hundreds of needle sticks during its lifetime.

In certain embodiments, the anti-CD33×anti-CD3 bispecific antibody construct is administered to the patient as a short intravenous infusion, which is typically an infusion of a small volume (e.g. 20 mL to 100 mL) administered over a period of, at most three hours. Preferably, each of the doses of the bispecific antibody construct administered to the patient during the initiation cycle and/or the maintenance cycle according to the methods of the invention is administered as an intravenous infusion of about 30 min to about 3 hours, about 30 min to about 90 min, or about 30 min to about 60 min.

In embodiments in which the bispecific antibody construct is infused, an infusion pump may be used to infuse the bispecific antibody construct into a patient's circulatory system. The pump is generally used intravenously, although arterial and epidural infusions with pumps are also possible. The solution for infusion may be prepared in bags for IV infusion and delivered through infusion lines. Pump systems for delivering intravenous infusions are known in the art. It is also possible that infusions are administered using only the pressure supplied by gravity.

In certain embodiments, the pharmaceutical compositions comprise a therapeutically effective amount of the anti-CD33×anti-CD3 bispecific antibody construct and one or more excipients. Excipients can be used for a wide variety of purposes, such as adjusting physical, chemical, or biological properties of formulations, such as adjustment of viscosity, and/or to stabilize such formulations against degradation and spoilage e.g. due to stresses that occur during manufacturing, shipping, storage, pre-use preparation, and administration.

In some embodiments, the pharmaceutical composition comprising a therapeutically effective amount of an anti-CD33×anti-CD3 bispecific antibody construct to be administered to a patient according to the methods of the invention comprises a buffer. Buffers are used to maintain the composition at physiological pH or at a slightly lower pH, typically within a pH range from about 4.0 to about 6.5. Suitable buffers include, but are not limited to, glutamate, aspartate, acetate, Tris, citrate, histidine, succinate, and phosphate buffers. In certain embodiments, the pharmaceutical composition administered according to the methods described herein comprises a glutamate buffer, particularly L-glutamate buffer. Pharmaceutical compositions comprising a glutamate buffer can have a pH of about 4.0 to about 5.5, a pH of about 4.0 to about 4.4, or a pH of about 4.2 to about 4.8.

The pharmaceutical composition comprising a therapeutically effective amount of an anti-CD33×anti-CD3 bispecific antibody construct may further comprise a surfactant. The term “surfactant” as used herein refers to a substance that functions to reduce the surface tension of a liquid in which it is dissolved. Surfactants can be included in pharmaceutical compositions for a variety of purposes including, for example, to prevent or control aggregation, particle formation and/or surface adsorption in liquid formulations or to prevent or control these phenomena during the lyophilization and/or reconstitution process in lyophilized formulations. Surfactants include, for example, amphipathic organic compounds that exhibit partial solubility in both organic solvents and aqueous solutions. General characteristics of surfactants include their ability to reduce the surface tension of water, reduce the interfacial tension between oil and water and also form micelles. Surfactants that may be incorporated into the pharmaceutical compositions used in the methods of the invention include both non-ionic and ionic surfactants. Suitable non-ionic surfactants include, but are not limited to, alkyl poly (ethylene oxide), alkyl polyglucosides, such as octyl glucoside and decyl maltoside, fatty alcohols, such as cetyl alcohol and oleyl alcohol, cocamide MEA, cocamide DEA, and cocamide TEA. Specific examples of non-ionic surfactants include the polysorbates including, for example, polysorbate 20, polysorbate 28, polysorbate 40, polysorbate 60, polysorbate 65, polysorbate 80, polysorbate 81, polysorbate 85 and the like; the poloxamers including, for example, poloxamer 188, also known as poloxalkol or poly(ethylene oxide)-poly(propylene oxide), poloxamer 407 or polyethylene-polypropylene glycol and the like, and polyethylene glycol (PEG). Suitable ionic surfactants include, for example, anionic, cationic and zwitterionic surfactants. Anionic surfactants include, but are not limited to, sulfonate-based or carboxylate-based surfactants such as soaps, fatty acid salts, sodium dodecyl sulfate (SDS), ammonium lauryl sulfate and other alkyl sulfate salts. Cationic surfactants include, but are not limited to, quaternary ammonium-based surfactants such as cetyl trimethylammonium bromide (CTAB), other alkyltrimethylammonium salts, cetyl pyridinium chloride, polyethoxylated tallow amine (POEA) and benzalkonium chloride. Zwitterionic or amphoteric surfactants include, for example, dodecyl betaine, dodecyl dimethylamine oxide, cocamidopropyl betaine and coco ampho glycinate. In certain embodiments, the pharmaceutical compositions administered according to the methods described herein comprise a non-ionic surfactant. In one embodiment, the non-ionic surfactant is polysorbate 20. In another embodiment, the non-ionic surfactant is polysorbate 80.

In certain embodiments, the pharmaceutical composition comprising a therapeutically effective amount of an anti-CD33×anti-CD3 bispecific antibody construct further comprises a stabilizing agent. As used herein, the term “stabilizing agent” refers to an excipient that stabilizes the native conformation of the polypeptide or antibody construct and/or prevents or reduces the physical or chemical degradation of the polypeptide or antibody construct. Suitable stabilizing agents include, but are not limited to, polyols (e.g. sorbitol, glycerol, mannitol, xylitol, maltitol, lactitol, erythritol and threitol), sugars (e.g., fructose, glucose, glyceraldehyde, lactose, arabinose, mannose, xylose, ribose, rhamnose, galactose maltose, sucrose, trehalose, sorbose, sucralose, melezitose and raffinose), and amino acids (e.g., glycine, methionine, proline, lysine, arginine, histidine, or glutamic acid). In some embodiments, the pharmaceutical composition comprises a sugar as a stabilizing agent. In these and other embodiments, the sugar is sucrose.

Exemplary pharmaceutical compositions comprising bispecific antibody constructs, including anti-CD33×anti-CD3 bispecific antibody constructs, are described in WO 2018/141910, which is hereby incorporated by reference in its entirety. In certain embodiments, a pharmaceutical composition useful for the treatment of myeloid leukemia according to the methods described herein comprises about 0.5 mg/ml to about 2 mg/ml of an anti-CD33×anti-CD3 bispecific antibody construct, about 5 mM to about 20 mM L-glutamic acid, about 0.005% to about 0.015% weight/volume (w/v) polysorbate (e.g. polysorbate 20 or polysorbate 80), and about 7% to about 12% (w/v) sucrose. In other embodiments, the pharmaceutical composition comprises about 0.5 mg/ml to about 1 mg/ml of an anti-CD33×anti-CD3 bispecific antibody construct, about 8 mM to about 12 mM L-glutamic acid, about 0.008% to about 0.012% (w/v) polysorbate (e.g. polysorbate 20 or polysorbate 80), and about 8% to about 10% (w/v) sucrose. The pH of these compositions is in the range of about 4.0 to about 4.4 (e.g., pH of about 4.0, about 4.1, about 4.2, about 4.3, or about 4.4).

Any of the pharmaceutical compositions comprising the anti-CD33×anti-CD3 bispecific antibody constructs described herein can be lyophilized and reconstituted with, e.g. sterile water for injection, prior to administration to the patient. Reconstitution volumes will depend on the protein content following lyophilization and the desired concentration of the bispecific antibody construct in the reconstituted solution, but may be from about 0.5 ml to about 5 ml. The solution following reconstitution can be further diluted with a diluent (e.g. saline and/or intravenous solution stabilizer (IVSS)) prior to administration to the patient as appropriate in order to administer the doses described herein according to the methods of the invention.

Any of the anti-CD33×anti-CD3 bispecific antibody constructs described herein, including the single chain antibody constructs described in Table 6, can be incorporated into any of the pharmaceutical compositions described above and administered to a patient according to the methods described herein. In a preferred embodiment, the anti-CD33×anti-CD3 bispecific antibody construct comprises the amino acid sequence of SEQ ID NO: 125. In another preferred embodiment, the anti-CD33×anti-CD3 bispecific antibody construct comprises the amino acid sequence of SEQ ID NO: 126.

The present invention also includes kits for treating myeloid leukemia in a patient in need thereof. In one embodiment, the kit comprises a pharmaceutical composition of an anti-CD33×anti-CD3 bispecific antibody construct described herein and packaging material that provides instructions regarding the use of the pharmaceutical compositions. The pharmaceutical composition of the kit may be present in a container, such as a vial. The pharmaceutical composition may be provided as a solution, suspension, gel, emulsion, solid, crystal, or as a dehydrated or lyophilized powder. In embodiments in which the pharmaceutical composition is provided as a lyophilized powder, the kit may also comprise diluents (e.g. sterile water for injection, saline, phosphate-buffer saline, formulation buffer) necessary to reconstitute the pharmaceutical composition as well as instructions for preparing the composition for administration. In certain embodiments, the kits may further comprise one or more vials of intravenous solution stabilizer (IVSS) and instructions for using the IVSS for pre-treatment of IV bags prior to dilution of the pharmaceutical composition for delivery to the patient. IVSS does not contain an active pharmaceutical ingredient and is typically a buffered, preservative-free solution. In one embodiment, IVSS comprises citric acid (e.g. 20-30 mM), lysine hydrochloride (e.g. 1-3M), and polysorbate 80 (0.05%-0.15% (w/v)) at pH 7.0. In a particular embodiment, IVSS comprises 25 mM citric acid, 1.25M lysine hydrochloride, and 0.1% (w/v) polysorbate 80 at pH 7.0.

The following examples, including the experiments conducted and the results achieved, are provided for illustrative purposes only and are not to be construed as limiting the scope of the appended claims.

### EXAMPLES

### Example 1. Phase 1 Study Evaluating the Safety, Tolerability, Pharmacokinetics, Pharmacodynamics and Efficacy of AMG 673 in Patients with Relapsed/Refractory Acute Myeloid Leukemia

AMG 673 is a half-life extended (HLE) BiTE® (bispecific T-cell engager) construct that binds both CD33 and CD3 and comprises a single chain IgG Fc region. The amino acid sequence of AMG 673 is set forth in SEQ ID NO: 125. AMG 673 redirects T-cells toward CD33+ cells, with the induced proximity leading to T-cell mediated cytotoxicity against acute myeloid leukemia (AML) blasts. The objectives of this Phase 1 study were to evaluate the safety, tolerability, pharmacokinetic (PK), pharmacodynamic (PD), and preliminary efficacy of AMG 673 in adult patients≥18 years of age with relapsed/refractory (R/R) AML. Anti-leukemia activity of AMG 673 was evaluated by the number and proportion of patients who respond to treatment with AMG 673, duration of response, time to progression, and time to response. Response was defined as any of the following: complete remission (CR), CR with incomplete hematologic recovery (CRi), or morphologic leukemia-free state, all in accordance with the Revised International Working Group response criteria, or CR with partial hematologic recovery (CRh).

After signing informed consent, patients entered the screening period (approx. 14 days), during which eligibility of the patients was assessed. Eligible patients included adults≥18 years of age who have AML as defined by World Health Organization Classification (see, e.g., Arber et al., Blood, Vol. 127: 2391-2405, 2016) persisting or recurring following 1 or more treatment courses and have more than 5% myeloblasts in bone marrow.

AMG 673 was administered as a short intravenous (IV) infusion (approximately 30 minutes to 3 hours) at doses ranging from 0.05 μg to 360 μg on day 1 (D1) and day 5 (D5) during each 14-day treatment cycle in adult patients with R/R AML. Dose levels (dose per each infusion) for the dose-escalation cohorts were: 0.05 μg, 0.15 μg, 0.45 μg, 1.5 μg, 4.5 μg, 7 μg, 9 μg, 18 μg, 36 μg, 72 μg, 110 μg, 160 μg, 240 μg, and 360 μg. All patients were pre-treated with an 8 mg dose of IV dexamethasone 1 hour prior to day 1 and day 5 infusions of AMG 673. Patients received treatment cycles of AMG 673 until disease progression or unacceptable toxicities. T-cell activation, cytokine, and AMG 673 levels in patients' blood were evaluated by validated assays. Results were summarized descriptively by the dosing cohorts and the potential associations between PK, PD, safety, and preliminary efficacy were evaluated.

Ten cohorts have been completed to date. Thirty patients were enrolled in the 10 cohorts and were treated with AMG 673 (dose range, 0.05 μg-72 μg IV per dose). The median age was 67.5 (range: 25.0-84.0) years; 20/30 (67%) patients had received ≥4 prior anti-AML treatments, baseline myelosuppression at study entry was common (grade≥3 neutropenia 21/30 [70%], thrombocytopenia 25/30 [83%], leukopenia 14/30 [47%]), and 7/30 (23%) patients had undergone hematopoietic stem cell transplant (HSCT) before enrolling in the study.

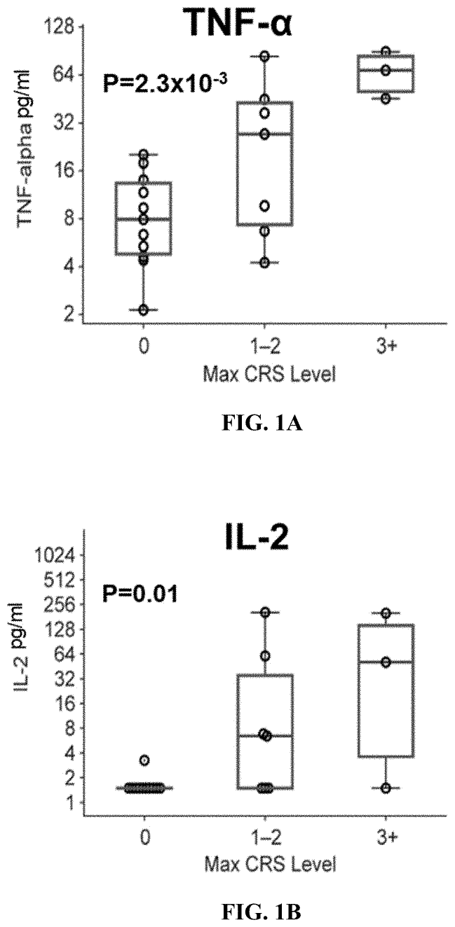

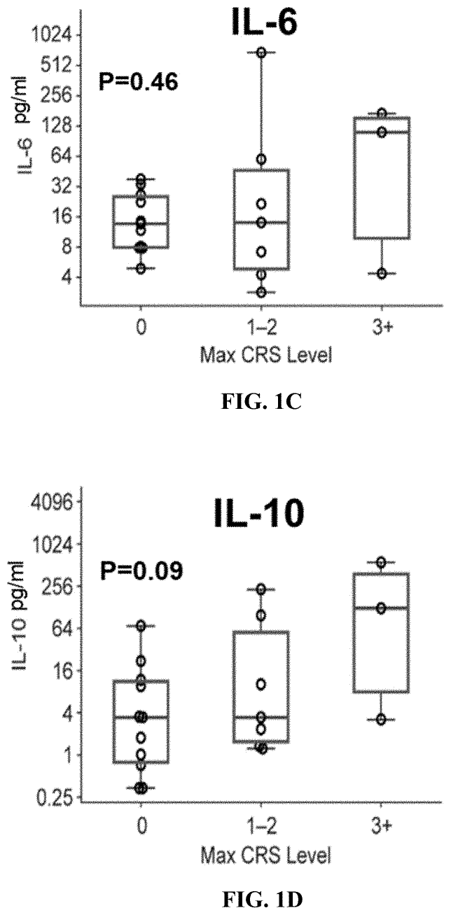

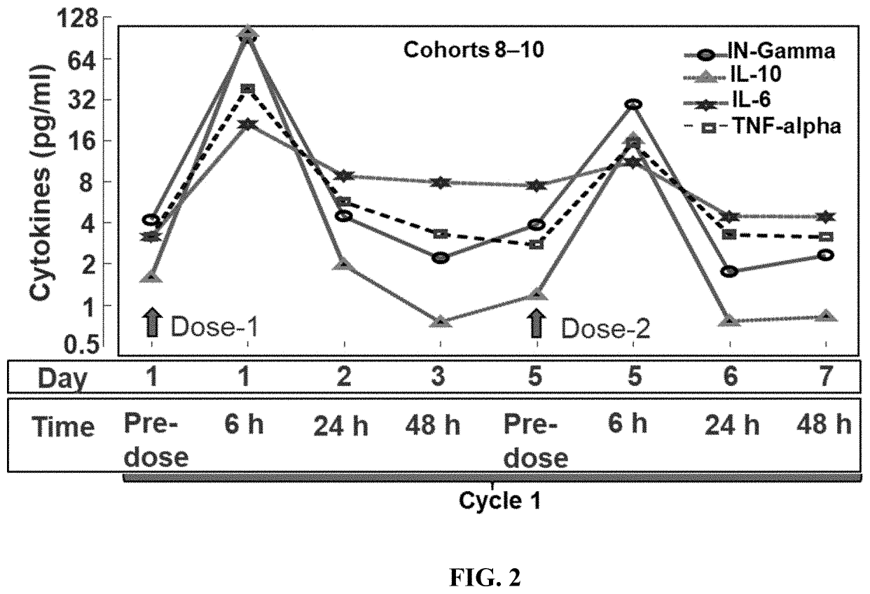

Patients received a median of 1.5 (range: 1.0-6.0) cycles of AMG 673; 27/30 (90%) patients discontinued treatment due to disease progression (n=21), patient request (n=2), protocol specified criteria (n=2), or adverse events (AEs; n=2). A total of 3 patients were receiving AMG 673 at the time of data analysis. The most common treatment-related AE was cytokine release syndrome (CRS) reported in 15/30 (50%) patients (grade 1, n=6; grade 2, n=5; grade 3, n=4; no grade 4 CRS). Treatment-related serious AEs were reported in 11/30 (37%) patients, and 15/30 (50%) patients experienced treatment-related AEs of grade≥3, with most common being abnormal hepatic enzymes (n=5, 17%), CRS (n=4, 13%), leukopenia (n=4, 13%), thrombocytopenia (n=2, 7%), and febrile neutropenia (n=2, 7%). Two deaths, unrelated to AMG 673, were reported on days 19 and 28 after the last dose. Based on exploratory assessments, severity of CRS correlated with serum cytokine levels, higher leukemic burden at baseline, and exposure to AMG 673. Specifically, severity of CRS correlated with maximum levels of cytokines (tumor necrosis factor-alpha (TNF-α), interleukin-2 (IL-2), interleukin-6 (IL-6), and interleukin-10 (IL-10)) in the serum in the first cycle (FIGS. 1A-1D). Grade 3 CRS was associated with the highest levels of TNF-α, IL-2, IL-6, and IL-10 in the serum. CRS is an acute toxicity and the onset of clinical symptoms correlated with timing of cytokine release in circulation. Maximal levels of cytokines were observed in serum 6 hours post-administration of 18 μg, 36 μg, or 72 μg of AMG 673 in cohorts 8-10 (FIG. 2).

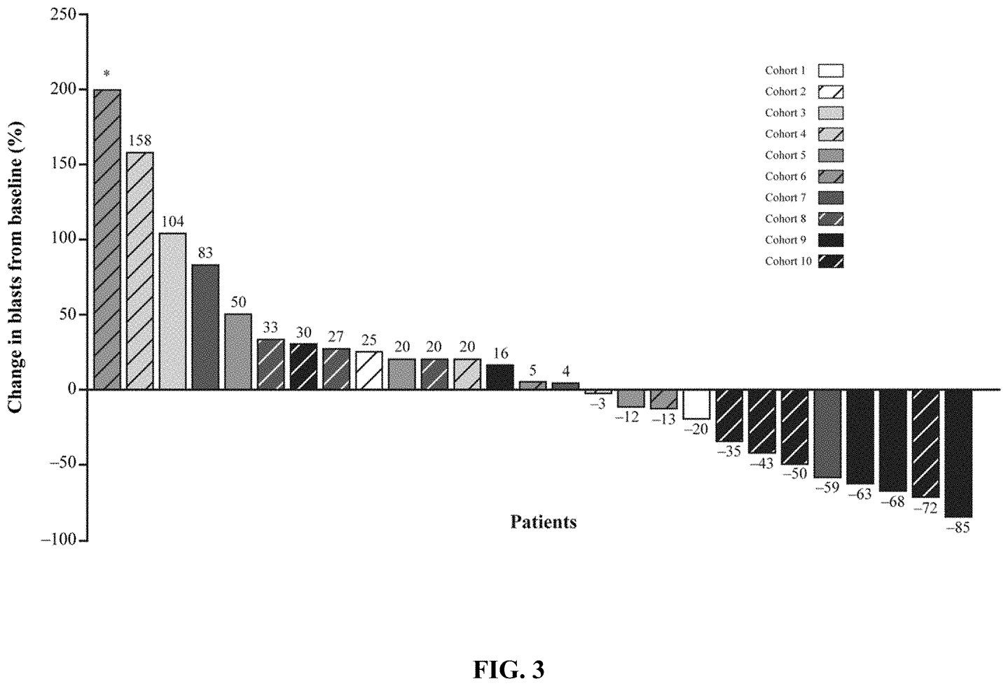

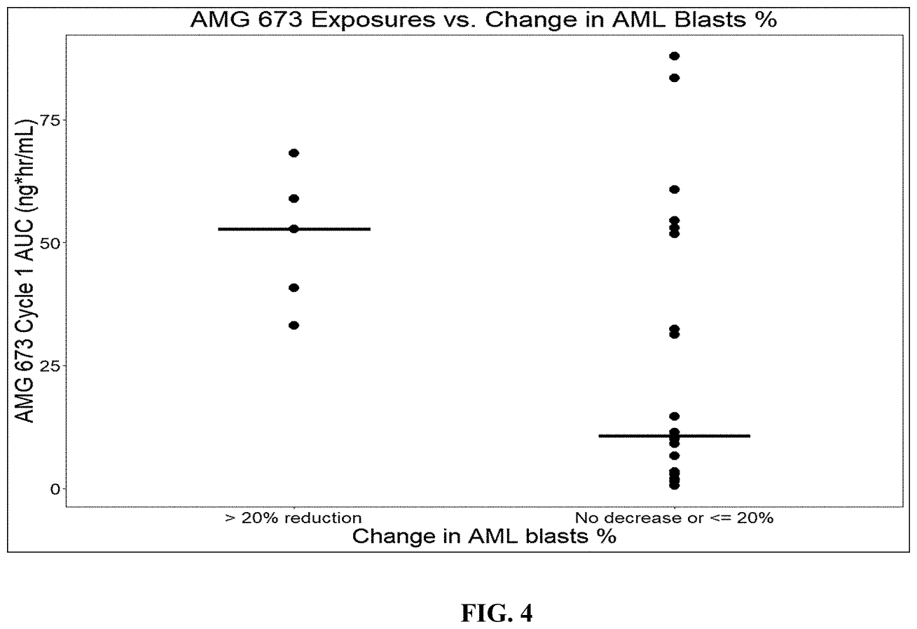

Assessment of bone marrow in treated patients showed a decrease in blasts in 12/27 (44%) evaluable patients, of which 6 patients experienced ≥50% reduction in blasts compared with baseline (FIG. 3). One patient achieved complete remission with incomplete hematologic recovery (CRi) with 85% reduction in bone marrow blasts at a dose of 36 μg and bridged to allogeneic hematopoietic stem cell transplant. Reduction in blast numbers were generally observed in patients with higher serum exposure to AMG 673 during cycle 1 (FIG. 4).

Exposures of AMG 673 observed for dosing cohorts up to 72 μg when administered on D1 and D5 of a 14-day treatment cycle in the dose escalation phase are shown in Table 7. Dose related increases in Cmax and AUC were observed following AMG 673 infusions (Table 7 below). Preliminary half-life estimates for AMG 673 were longer than those observed for a canonical anti-CD33×anti-CD3 BITE® molecule (i.e. molecule lacking the single chain Fc region). Alternative dosing regimens to increase exposure of AMG 673 in cycle 1 are evaluated in separate cohorts of patients (see Example 2).

TABLE 7
Observed AMG 673 Pharmacokinetic Parameters in AML Patients

| AMG 673 Dose | Mean AUC | Mean Cₘₐₓ |
|---|---|---|
| (μg/dose) | (ng*hr/ml) | (ng/ml) |
| 0.05 | 0.62 | 0.01 |
| 0.15 | 1.67 | 0.03 |
| 0.45 | 2.08 | 0.04 |
| 1.50 | 2.31 | 0.02 |
| 4.50 | 8.17 | 0.26 |
| 7.0 | 28.2 | 0.62 |
| 9.0 | 9.10 | 0.87 |
| 18.0 | 29.2 | 1.11 |
| 36.0 | 47.0 | 1.85 |
| 72.0 | 65.2 | 2.68 |

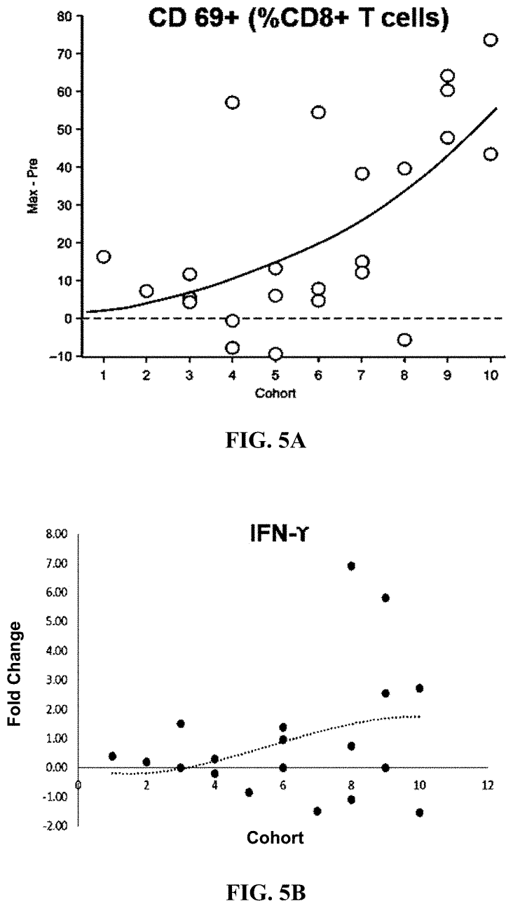

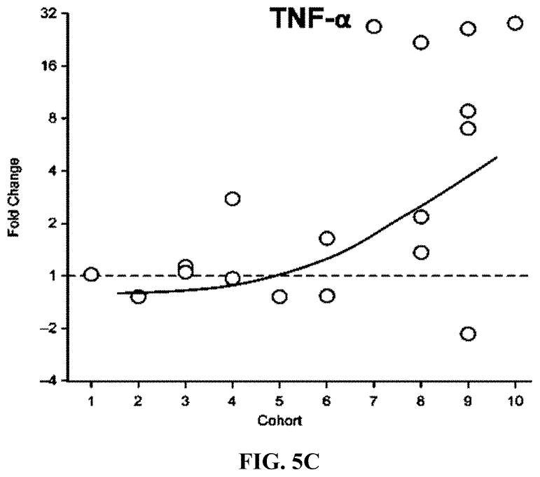

Upregulation of T-cell activation markers CD25 and CD69 on T-cell subsets and cytokine release post-infusion were observed at higher doses (FIGS. 5A-5C). Preliminary associations between AMG 673 exposures, T-cell activation, safety, and clinical response have been evaluated. T-cell activation as measured by CD69 expression in CD8+ T cells was observed at higher exposures (FIG. 5A). Increased levels of cytokines (IFN-γ and TNF-α) in serum were observed at higher exposures (FIGS. 5B and 5C).

Preliminary data of AMG 673 dosed up to 72 μg provides early evidence of the molecule's acceptable safety profile, drug tolerability, and anti-leukemic activity. An association was observed between PK/PD relationships that were consistent with the biological activity of AMG 673 with a decrease in AML blasts in bone marrow observed at higher AMG 673 exposures. These preliminary results support further dose escalation of AMG 673 in patients with R/R AML.

Following the data snapshot described above, which included 30 patients enrolled in the first ten cohorts, an additional 8 patients in cohorts 11 and 12 (4 patients in each cohort) were treated with AMG 673. Dose-limiting toxicities in 2 out of 4 treated patients in cohort 11 (110 μg dose) led to the identification of 72 μg (cohort 10) as the maximum tolerated starting dose (MTD-1). A dose step was initiated in cohort 12 to mitigate severity of CRS and allow for further dose escalation. The four patients enrolled in cohort 12 received a 36 μg dose of AMG 673 on day 1 (D1) and a 72 μg dose of AMG 673 on day 4 (D4) during each 14-day treatment cycle. As of the second data snapshot, 38 patients have been treated with AMG 673 in 12 different dosing cohorts. Demographics and baseline characteristics for these patients are described in Table 8 below.

TABLE 8
Patient demographics and baseline characteristics

| Characteristics | N = 38 | |
|---|---|---|
| Median age, years | 67.5 | (25-84) |
| Male, n (%) | 20 | (53) |
| AML type, n (%) | | |
| AML with recurrent genetic abnormalities | 15 | (39) |
| AML, not otherwise specified | 12 | (32) |
| AML with myelodysplasia-related changes | 8 | (21) |
| Therapy-related myeloid neoplasms | 3 | (8) |
| Baseline myelosuppression (≥ grade 3), n (%) | | |
| Thrombocytopenia | 32 | (84) |
| Neutropenia | 26 | (68) |
| Leukopenia | 17 | (45) |
| Prior therapies, n (%) | | |
| 1 | 4 | (10) |
| 2 | 3 | (8) |
| 3 | 6 | (16) |
| ≥4 | 25 | (66) |
| Prior hematopoietic stem cell transplantation, n (%) | 7 | (18) |

Treatment-related AEs were reported in 34/38 (90%) patients. CRS was the most common treatment-related AE observed in 24/38 (63%) patients. Treatment-related serious AEs were reported in 26/38 (68%) patients, and 20/38 (53%) patients experienced treatment-related AEs of grade≥3. Reduction in blasts was observed in 16/38 (42%) patients, of which 6 patients experienced ≥50% reduction in blasts compared with baseline. One patient from cohort 9 (36 μg dose) achieved CRi with 85% reduction in bone marrow blasts and bridged to allogeneic hematopoietic stem cell transplantation. Dose-related increases in AMG 673 exposures were observed across the tested dose range of 0.05 μg to 110 including the dose-step (36 μg to 72 μg) in cohort 12. Decreases in AML blasts in bone marrow were observed at higher AMG 673 exposures.

These additional data for AMG 673 identified the starting dose limiting level of 110 μg leading to the introduction of a step dose schedule to enable further dose escalation. Preliminary assessment shows an acceptable safety profile with evidence of anti-leukemic activity. These updated results support further evaluation of altered dosing schedules and continued dose escalation of AMG 673 in patients with R/R AML.

### Example 2. Phase 1 Study Evaluating the Safety, Tolerability, and Efficacy of AMG 673 in Relapsed/Refractory Acute Myeloid Leukemia Patients

The main objectives of this study are to evaluate the safety, tolerability, anti-leukemic activity, and pharmacokinetics (PK) of AMG 673, an HLE BITE® construct (SEQ ID NO: 125), in adult patients with relapsed and/or refractory AML when administered on a daily frequency during the initial cycle of treatment. As described in Example 1, a reduction in bone marrow blasts was generally observed at higher exposures of AMG 673 during cycle 1. Because the observed AMG 673 exposures were lower than expected for the first set of dosing cohorts based on allometric scaling of PK parameters in cynomolgus monkeys, the dosing schedule was adjusted to minimize exposure interruptions and to increase exposure to an efficacious dose as rapidly as possible during cycle 1 without triggering adverse effects, such as CRS.

This study is an open-label, phase 1, sequential dose escalation study. AMG 673 is evaluated as a short-term intravenous (IV) infusion in adult patients with relapsed and/or refractory AML. The dose-escalation cohorts estimate the maximum tolerated dose (MTD), safety, tolerability, PK, and pharmacodynamics (PD) of AMG 673. The dose levels (dose per infusion) for the dose-escalation cohorts are as follows: 72 μg, 110 μg, 150 μg, 180 μg, 240 μg, 360 μg, 480 μg and higher if MTD is not reached. At the completion of the dose escalation cohorts, additional patients are enrolled in a dose expansion cohort to gain further clinical experience, safety, and efficacy data in patients with AMG 673.

After written informed consent is obtained, patients enter the screening period (approx. 14 days), during which eligibility of the patients is assessed. Eligible patients include adults≥18 years of age who have AML as defined by World Health Organization Classification (see, e.g., Arber et al., Blood, Vol. 127: 2391-2405, 2016) persisting or recurring following 1 or more treatment courses and have more than 5% myeloblasts in bone marrow. Patients deemed eligible are enrolled in the trial. Treatment begins on day 1 (cycle 1 day 1; D1) when the first IV infusion of AMG 673 is administered to a patient.

The starting dose for the first cohort is 72 μg administered daily as short-term IV infusions (approximately 30 minutes to 3 hours) on a 14-day cycle (e.g. each dose administered on D1, D2, D3, D4, D5, D6, D7, D8, D9, D10, D11, D12, D13, and D14) followed by administration of a second cycle with the dose administered twice per week starting the following day for a 14-day treatment cycle (e.g. each dose administered on D1, D4, D8, and D11). A lower starting dose (e.g. 18 μg or 36 μg) may be used for up to the first two doses. Cycles 3 and beyond are identical to cycle 2. Dosing for cohorts following cohort 1, as described in more detail below, is recommended by the Dose Level Review Team (DLRT) and can be adjusted based on emerging data. All patients are pre-treated with an 8-mg dose of IV dexamethasone 1 hour prior to the first dose of AMG 673 on day 1, prior to each step-up dose of AMG 673 in cycle 1, and prior to all doses of AMG 673 in cycle 2 and beyond. There is no treatment free interval between cycle 1 and cycle 2 (i.e. cycle 2, day 1 is the day following cycle 1, day 14). In treatment cycles following cycle 2, a treatment-free interval of 1 to 14 days may be employed between treatment cycles depending on treatment response and recovery of blood counts.

Patients will be dosed in each of the cohorts as follows:
Cohort 1: 72 μg once each day for 14 days (e.g. dosed on each of D1 to D14) followed immediately by 72 μg twice per week for 14 days (e.g. cycle 2 dosed on each of D1, D4, D8, and D11)
Cohort 2: 72 μg once each day for 2 days (e.g. dosed on each of D1 and D2), followed by 110 μg once each day for 12 days (e.g. dosed on each of D3 to D14), followed immediately by 110 μg twice per week for 14 days (e.g. cycle 2 dosed on each of D1, D4, D8, and D11)
Cohort 3: 72 μg once each day for 2 days (e.g. dosed on each of D1 and D2), followed by 110 μg once each day for 2 days (e.g. dosed on each of D3 and D4), followed by 150 μg once each day for 10 days (e.g. dosed on each of D5 to D14), followed immediately by 150 μg twice per week for 14 days (e.g. cycle 2 dosed on each of D1, D4, D8, and D11)
Cohort 4: 72 μg once each day for 2 days (e.g. dosed on each of D1 and D2), followed by 110 μg once each day for 2 days (e.g. dosed on each of D3 and D4), followed by 150 μg once each day for 2 days (e.g. dosed on each of D5 and D6), followed by 180 μg once each day for 8 days (e.g. dosed on each of D7 to D14), followed immediately by 180 μg twice per week for 14 days (e.g. cycle 2 dosed on each of D1, D4, D8, and D11)
Cohort 5: 72 μg once each day for 2 days (e.g. dosed on each of D1 and D2), followed by 110 μg once each day for 2 days (e.g. dosed on each of D3 and D4), followed by 150 μg once each day for 2 days (e.g. dosed on each of D5 and D6), followed by 180 μg once each day for 2 days (e.g. dosed on each of D7 and D8), followed by 240 μg once each day for 6 days (e.g. dosed on each of D9 to D14), followed immediately by 240 μg twice per week for 14 days (e.g. cycle 2 dosed on each of D1, D4, D8, and D11)
Cohort 6: 72 μg once each day for 2 days (e.g. dosed on each of D1 and D2), followed by 110 μg once each day for 2 days (e.g. dosed on each of D3 and D4), followed by 150 μg once each day for 2 days (e.g. dosed on each of D5 and D6), followed by 180 μg once each day for 2 days (e.g. dosed on each of D7 and D8), followed by 240 μg once each day for 2 days (e.g. dosed on each of D9 and D10), followed by 360 μg once each day for 4 days (e.g. dosed on each of D11 to D14), followed immediately by 360 μg twice per week for 14 days (e.g. cycle 2 dosed on each of D1, D4, D8, and D11)
Cohort 7: 72 μg once each day for 2 days (e.g. dosed on each of D1 and D2), followed by 110 μg once each day for 2 days (e.g. dosed on each of D3 and D4), followed by 150 μg once each day for 2 days (e.g. dosed on each of D5 and D6), followed by 180 μg once each day for 2 days (e.g. dosed on each of D7 and D8), followed by 240 μg once each day for 2 days (e.g. dosed on each of D9 and D10), followed by 360 μg once each day for 2 days (e.g. dosed on each of D11 and D12), followed by 480 μg once each day for 2 days (e.g. dosed on each of D13 and D14), followed immediately by 480 μg twice per week for 14 days (e.g. cycle 2 dosed on each of D1, D4, D8, and D11)

Each cohort enrolls up to 4 evaluable patients. There is at least a 7-day (168-hour) interval between the start of treatment of the first and second patient in each cohort (i.e. at the same dose and schedule). On day 7 of this interval, all available safety and laboratory data for the treated patient is evaluated and a written confirmation on occurrence/non-occurrence of a dose-limiting toxicity (DLT) is made. Enrollment for the next patient in the cohort is opened only after receipt of this confirmation. Dose escalations continue until either (i) no DLTs are observed on study or (ii) DLTs are observed on study and either a minimum of 6 patients are treated at a dose level or a maximum of 40 patients are enrolled.

Anti-leukemia activity of AMG 673 is evaluated by the number and proportion of patients who respond to treatment with AMG 673, duration of response, time to progression, and time to response. Disease response assessments are based upon review of cytogenetics, bone marrow aspirates/biopsies, and peripheral blood count. Response is defined as any of the following: complete remission (CR), CR with incomplete hematologic recovery (CRi), or morphologic leukemia-free state, all in accordance with the Revised International Working Group response criteria, or CR with partial hematologic recovery (CRh) as described below.

### CR

less than 5% blasts in the bone marrow
absence of blasts with Auer rods
absence of extramedullary disease
absolute neutrophil count (ANC)≥1,000/μl
platelet count≥100,000/μl
independence of red cell transfusions

### CRi

all CR criteria except for incomplete recovery of peripheral blood counts (residual neutropenia [<1,000/μl] or thrombocytopenia [<100,000/μl])

### Morphologic Leukemia-Free State

less than 5% myeloblasts in the bone marrow
absence of blasts with Auer rods
absence of extramedullary disease
no hematologic recovery required

### CRh

less than 5% blasts in the bone marrow
no evidence of disease
partial recovery of peripheral blood counts: platelet count>50,000/μl, and
ANC>500/μl
no extramedullary disease

Adverse event and serious adverse event as well as disease-related event assessments are made throughout the study and are evaluated and recorded in the source documents. The severity of all events is graded according to CTCAE, version 4.0 unless specified otherwise. Exception: CRS is graded according to the adopted grading system referenced in Lee et al., Blood, Vol. 124: 188-195, 2014.

Five patients were enrolled in cohort 1. Prior to enrollment, the starting dose was adjusted to 36 μg for the first two days and then increased to 72 μg for the remainder of cycle 1 (Table 10). Thus, patients in cohort 1 received AMG 673 as a short-term IV infusion (approximately 30 minutes to 3 hours) according to the following dosing regimen: 36 μg once each day for 2 days (e.g. dosed on each of D1 and D2 in cycle 1), followed by 72 μg once each day for 12 days (e.g. dosed on each of D3 to D14 in cycle 1), followed immediately by 72 μg twice per week for 14 days (e.g. cycle 2 dosed on each of D1, D4, D8, and D11). Cycle 3 and subsequent cycles were identical to cycle 2. Two patients withdrew from the study following the first dose of 36 μg on cycle 1, day 1. One such patient had a grade 2 CRS and the other such patient had a grade 3 CRS, including grade 3 transaminitis, which was classified as a DLT. Only grade 1 CRS was observed during cycle 1 in the remaining three patients enrolled in cohort 1. Reduction in blasts was observed in 3/5 patients (60%) with 2 patients exhibiting a greater than 50% reduction. See Table 9 below.

TABLE 9
Change in Bone Marrow Blasts in AML Patients in Cohort 1

| | % Change in Bone Marrow |
|---|---|
| | Blasts from Baseline to |
| Patient | Best Response |
| Patient 1* | — |
| Patient 2* | 130.5 |
| Patient 3 | −17.6 |
| Patient 4 | −75.0 |
| Patient 5 | −99.1 |

*Patients 1 and 2 withdrew from study after receiving first dose and didn't receive the 72 μg target dose

Patient 4, a 67-year old female who had received two prior lines of therapy for AML, achieved a CRi with 75% reduction in bone marrow blasts and bridged to allogeneic hematopoietic stem cell transplantation following completion of cycle 1 of AMG 673 treatment. Patient 5, a 76-year old female who had received three prior lines of therapy for AML, achieved a CRh with 99.1% reduction in bone marrow blasts following completion of cycle 3 of AMG 673 treatment. This patient exhibited a 91% reduction from baseline in bone marrow blasts following completion of cycle 1 and achieved a CRi following completion of cycle 2 of AMG 673 treatment. Administration of a third cycle of AMG 673 treatment in this patient led to improved blood counts and achievement of a CRh. In addition, patient 5 did not experience CRS of any grade during cycles 2 and 3 of AMG 673 treatment and only experienced grade 1 CRS events during cycle 1.

Reduction in blast numbers was observed in patients with higher exposure to AMG 673. Based on available PK data from one patient, preliminary modeling results show that the terminal half-life of AMG 673 was approximately 2.5 days with daily (QD) dosing on day 14 as compared to a terminal half-life of approximately 1 day on day 5 with the D1/D5 dosing regimen described in Example 1.

The results from the first cohort of patients in this study suggest that exposures of AMG 673 are increased sufficiently within the first 14 days of treatment such that a greater proportion of patients exhibit a reduction in bone marrow blasts relative to patients receiving AMG 673 on the less frequent dosing schedule described in Example 1. The daily dosing of AMG 673 during cycle 1 appeared to result in an increased response rate as well as a reduction in severity of CRS events.

Based on the results in patients enrolled in cohort 1, the doses were adjusted in subsequent cohorts to allow for a lower starting dose on day 1 of cycle 1 and more rapid steps up to the target dose. Specifically, patients enrolled in cohort 2 receive AMG 673 as a short-term IV infusion (approximately 30 minutes to 3 hours) according to the following dosing regimen: 18 μg once a day for one day on day 1 (e.g. dosed on D1 in cycle 1), followed by 36 μg once a day for one day on day 2 (e.g. dosed on D2 in cycle 1), followed by 72 μg once a day for one day on day 3 (e.g. dosed on D3 in cycle 1), followed by 110 μg once each day for 11 days for the remainder of cycle 1 (e.g. dosed on each of D4 to D14 in cycle 1), followed immediately by 110 μg twice per week for 14 days (e.g. cycle 2 dosed on each of D1, D4, D8, and D11). A summary of the modified cycle 1 dosing in each dosing cohort is set forth in Table 10 below. Each dose is administered once per day (QD) on the indicated days in cycle 1 (14-day treatment cycle). Doses are given in micrograms (μg). All patients will be pre-treated with an 8-mg dose of IV dexamethasone 1 hour prior to AMG 673 dose on day 1 and prior to each step-up dose of AMG 673 during cycle 1. The duration of the dose steps may be extended up to 5 days, in which case the duration of the cycle can be extended from 14 days to 28 days. For example, the initial dose (18 μg) may be given just one day and the other dose steps may be given for 2 days before increasing to the next step dose or target dose.

TABLE 10
Cycle 1 Dosing by Cohort for Daily Dosing Schedule

| | D1 | D2 | D3 | D4 | D5 | D6 | D7 | D8 | D9 | D10 | D11 | D12 | D13 | D14 |
|---|---|---|---|---|---|---|---|---|---|---|---|---|---|---|
| Cohort 1 | 36 | 36 | 72 | 72 | 72 | 72 | 72 | 72 | 72 | 72 | 72 | 72 | 72 | 72 |
| Cohort 2 | 18 | 36 | 72 | 110 | 110 | 110 | 110 | 110 | 110 | 110 | 110 | 110 | 110 | 110 |
| Cohort 3 | 18 | 36 | 72 | 110 | 160 | 160 | 160 | 160 | 160 | 160 | 160 | 160 | 160 | 160 |
| Cohort 4 | 18 | 36 | 72 | 110 | 160 | 240 | 240 | 240 | 240 | 240 | 240 | 240 | 240 | 240 |
| Cohort 5 | 18 | 36 | 72 | 110 | 160 | 240 | 360 | 360 | 360 | 360 | 360 | 360 | 360 | 360 |
| Cohort 6 | 18 | 36 | 72 | 110 | 160 | 240 | 360 | 480 | 480 | 480 | 480 | 480 | 480 | 480 |

Cycle 2 and all subsequent cycles consist of four AMG 673 infusions on day 1, day 4, day 8, and day 11 of a 14-day cycle at the target dose (maximum dose received in cycle 1) (see Table 11 below where doses are presented in micrograms (μg)). The cycle 1 schedule may be repeated once before cycle 2 schedule is initiated if the patient will likely gain additional benefit. If cycle 1 is not repeated, cycle 2 should begin immediately after cycle 1 with no dose free interval (e.g. day 15 after first dose of AMG 673 in cycle 1=day 1 of cycle 2). Based on emerging PK, safety, and PD data, the number of infusions in cycle 2 and subsequent cycles may be decreased such that AMG 673 is administered at a weekly frequency (e.g. on day 1 and day 8 of each 14-day treatment cycle). Dexamethasone 8 mg IV is administered 1 hour prior to the first dose of AMG 673 on day 1 of cycle 2 and subsequent cycles.

TABLE 11
Cycle 2 Dosing by Cohort¹

| | D1 | D2 | D3 | D4 | D5 | D6 | D7 | D8 | D9 | D10 | D11 | D12 | D13 | D14 |
|---|---|---|---|---|---|---|---|---|---|---|---|---|---|---|
| Cohort 1 | 72 | Z | Z | 72 | Z | Z | Z | 72 | Z | Z | 72 | Z | Z | Z |
| Cohort 2 | 110 | | | 110 | | | | 110 | | | 110 | | | |
| Cohort 3 | 160 | | | 160 | | | | 160 | | | 160 | | | |
| Cohort 4 | 240 | | | 240 | | | | 240 | | | 240 | | | |
| Cohort 5 | 360 | | | 360 | | | | 360 | | | 360 | | | |
| Cohort 6 | 480 | | | 480 | | | | 480 | | | 480 | | | |

¹Cycle 3 and subsequent cycles have identical dosing to cycle 2

All publications, patents, and patent applications discussed and cited herein are hereby incorporated by reference in their entireties. It is understood that the disclosed invention is not limited to the particular methodology, protocols and materials described as these can vary. It is also understood that the terminology used herein is for the purposes of describing particular embodiments only and is not intended to limit the scope of the appended claims.

Those skilled in the art will recognize, or be able to ascertain using no more than routine experimentation, many equivalents to the specific embodiments of the invention described herein. Such equivalents are intended to be encompassed by the following claims.

What is claimed:

1. A method for treating myeloid leukemia in a patient in need thereof, comprising administering to the patient at least one initiation cycle and at least one maintenance cycle of a bispecific antibody construct that specifically binds to CD33 and CD3,
wherein the initiation cycle comprises administering the bispecific antibody construct at one or more doses of about 18 μg to about 480 μg at an interval of 1 day to 4 days for a first period of time,
wherein the maintenance cycle comprises administering the bispecific antibody construct at a dose of about 36 μg to about 480 μg once or twice every 7 days for a second period of time, and
wherein the maintenance cycle is administered after the initiation cycle.

2. The method of claim 1, wherein the first period of time is about 7 days to about 14 days.

3. The method of claim 1 or 2, wherein the initiation cycle comprises administering the dose of the bispecific antibody construct once per day for 7 days.

4. The method of claim 1 or 2, wherein the initiation cycle comprises administering the dose of the bispecific antibody construct once per day for 14 days.

5. The method of claim 1 or 2, wherein the initiation cycle comprises administering the dose of the bispecific antibody construct once every other day for 14 days.

6. The method of claim 1 or 2, wherein the initiation cycle comprises administering the dose of the bispecific antibody construct once every three days for 14 days.

7. The method of claim 1 or 2, wherein the initiation cycle comprises administering the dose of the bispecific antibody construct once every four days for 7 days or 14 days.

8. The method of any one of claims 1 to 7, wherein the initiation cycle comprises administering the bispecific antibody construct at one or more doses of about 100 μg to about 180 μg.

9. The method of any one of claims 1 to 7, wherein the initiation cycle comprises administering the bispecific antibody construct at one or more doses of about 18 μg to about 240 μg.

10. The method of any one of claims 1 to 9, wherein the dose of the bispecific antibody construct administered during the initiation cycle is the same at each interval.

11. The method of any one of claims 1 to 9, wherein the dose of the bispecific antibody construct administered during the initiation cycle increases at least once at one or more intervals during the cycle.

12. The method of claim 1 or 2, wherein the initiation cycle comprises administering the bispecific antibody construct at a first dose for one or more intervals and subsequently administering the bispecific antibody construct at a second dose for one or more intervals, wherein the second dose is greater than the first dose.

13. The method of claim 12, wherein the initiation cycle further comprises administering the bispecific antibody construct at a third dose for one or more intervals following administration of the second dose, wherein the third dose is greater than the second dose.

14. The method of claim 13, wherein the initiation cycle further comprises administering the bispecific antibody construct at a fourth dose for one or more intervals following administration of the third dose, wherein the fourth dose is greater than the third dose.

15. The method of claim 14, wherein the initiation cycle further comprises administering the bispecific antibody construct at a fifth dose for one or more intervals following administration of the fourth dose, wherein the fifth dose is greater than the fourth dose.

16. The method of claim 15, wherein the initiation cycle further comprises administering the bispecific antibody construct at a sixth dose for one or more intervals following administration of the fifth dose, wherein the sixth dose is greater than the fifth dose.

17. The method of any one of claims 12 to 16, wherein the interval is daily.

18. The method of any one of claims 12 to 17, wherein the first dose is about 18 μg to about 150 μg and the second dose is about 110 μg to about 240 μg.

19. The method of any one of claims 13 to 18, wherein the third dose is about 150 μg to about 360 μg.

20. The method of any one of claims 14 to 19, wherein the fourth dose is about 180 μg to about 480 μg.

21. The method of claim 12, wherein the first dose is about 36 μg and the second dose is about 72 μg, and wherein the interval is daily.

22. The method of claim 14, wherein the first dose is about 18 μg, the second dose is about 36 μg, the third dose is about 72 μg, and the fourth dose is about 110 μg, and wherein the interval is daily.

23. The method of claim 15, wherein the first dose is about 18 μg, the second dose is about 36 μg, the third dose is about 72 μg, the fourth dose is about 110 μg, and the fifth dose is about 160 μg, and wherein the interval is daily.

24. The method of claim 16, wherein the first dose is about 18 μg, the second dose is about 36 μg, the third dose is about 72 μg, the fourth dose is about 110 μg, the fifth dose is about 160 fig, and the sixth dose is about 240 μg, and wherein the interval is daily.

25. The method of any one of claims 1 to 24, wherein the dose of the bispecific antibody construct administered during the maintenance cycle is the same as the highest dose of the bispecific antibody construct administered during the initiation cycle.

26. The method of any one of claims 1 to 25, wherein the dose of the bispecific antibody construct administered during the maintenance cycle is about 110 μg to about 240 μg.

27. The method of any one of claims 1 to 25, wherein the dose of the bispecific antibody construct administered during the maintenance cycle is about 72 μg to about 360 μg.

28. The method of any one of claims 1 to 27, wherein the second period of time is about 14 days to about 28 days.

29. The method of any one of claims 1 to 28, wherein the maintenance cycle comprises administering the dose of the bispecific antibody construct once every 7 days for 14 days.

30. The method of any one of claims 1 to 28, wherein the maintenance cycle comprises administering the dose of the bispecific antibody construct once every 7 days for 28 days.

31. The method of any one of claims 1 to 28, wherein the maintenance cycle comprises administering the dose of the bispecific antibody construct twice every 7 days for 14 days.

32. The method of any one of claims 1 to 28, wherein the maintenance cycle comprises administering the dose of the bispecific antibody construct twice every 7 days for 28 days.

33. The method of any one of claims 1 to 32, wherein each of the doses of the bispecific antibody construct administered during the initiation cycle and/or the maintenance cycle is administered as an intravenous infusion of about 30 min to about 90 min.

34. The method of any one of claims 1 to 33, wherein the maintenance cycle is initiated the following day after completing the initiation cycle.

35. The method of any one of claims 1 to 34, wherein two or more maintenance cycles are administered to the patient.

36. The method of claim 35, wherein six to twelve maintenance cycles are administered to the patient.

37. The method of any one of claims 1 to 36, further comprising administering to the patient a glucocorticoid prior to administration of each dose of the bispecific antibody construct during the initiation cycle and/or maintenance cycle.

38. The method of claim 37, wherein the glucocorticoid is dexamethasone.

39. The method of any one of claims 1 to 38, wherein the myeloid leukemia is acute myeloid leukemia.

40. The method of claim 39, wherein the acute myeloid leukemia is relapsed and/or refractory acute myeloid leukemia.

41. The method of any one of claims 1 to 38, wherein the myeloid leukemia is chronic myeloid leukemia.

42. The method of any one of claims 1 to 41, wherein the patient has previously received one or more chemotherapy regimens.

43. The method of any one of claims 1 to 42, wherein the patient has received a hematopoietic stem cell transplant.

44. The method of any one of claims 1 to 43, wherein the bispecific antibody construct comprises, in an amino to carboxyl order:
(i) a first domain that specifically binds to human CD33 comprising a first immunoglobulin heavy chain variable region (VH1) comprising a CDRH1 having the sequence of SEQ ID NO: 10, a CDRH2 having the sequence of SEQ ID NO: 13, and a CDRH3 having the sequence of SEQ ID NO: 14, and a first immunoglobulin light chain variable region (VL1) comprising a CDRL1 having the sequence of SEQ ID NO: 6, a CDRL2 having the sequence of SEQ ID NO: 8, and a CDRL3 having the sequence of SEQ ID NO: 9;
(ii) a second domain that specifically binds to human CD3 comprising a second immunoglobulin heavy chain variable region (VH2) comprising a CDRH1 having the sequence of SEQ ID NO: 38, a CDRH2 having the sequence of SEQ ID NO: 44, and a CDRH3 having the sequence of SEQ ID NO: 49, and a second immunoglobulin light chain variable region (VL2) comprising a CDRL1 having the sequence of SEQ ID NO: 32, a CDRL2 having the sequence of SEQ ID NO: 33, and a CDRL3 having the sequence of SEQ ID NO: 36; and
(iii) a third domain comprising two Fc monomers, each monomer comprising an immunoglobulin hinge region, a CH2 domain, and a CH3 domain, wherein said two monomers are fused to each other via a peptide linker.

45. The method of claim 44, wherein VH1 comprises the sequence of SEQ ID NO: 28 and VL1 comprises the sequence of SEQ ID NO: 20.

46. The method of claim 44 or 45, wherein VH2 comprises the sequence of SEQ ID NO: 61 and VL2 comprises the sequence of SEQ ID NO: 59.

47. The method of any one of claims 44 to 46, wherein the first and second binding domains are single-chain variable fragment (scFv) domains.

48. The method of any one of claims 44 to 47, wherein the first binding domain comprises the sequence of SEQ ID NO: 91.

49. The method of any one of claims 44 to 48, wherein the second binding domain comprises the sequence of SEQ ID NO: 101.

50. The method of any one of claims 44 to 49, wherein each of said Fc monomers of the third domain comprises the sequence of SEQ ID NO: 109.

51. The method of any one of claims 44 to 50, wherein the third domain comprises the sequence of SEQ ID NO: 117.

52. The method of any one of claims 44 to 51, wherein the bispecific antibody construct is a single chain antibody construct.

53. The method of claim 52, wherein the bispecific antibody construct comprises the sequence of SEQ ID NO: 125.

54. A bispecific antibody construct that specifically binds to CD33 and CD3 for use in a method for treating myeloid leukemia in a patient in need thereof, wherein the method comprises administering to the patient at least one initiation cycle and at least one maintenance cycle of the bispecific antibody construct,
wherein the initiation cycle comprises administering the bispecific antibody construct at one or more doses of about 18 μg to about 480 μg at an interval of 1 day to 4 days for a first period of time,
wherein the maintenance cycle comprises administering the bispecific antibody construct at a dose of about 36 μg to about 480 μg once or twice every 7 days for a second period of time, and
wherein the maintenance cycle is administered after the initiation cycle.

55. The bispecific antibody construct of claim 54, wherein the first period of time is about 7 days to about 14 days.

56. The bispecific antibody construct of claim 54 or 55, wherein the initiation cycle comprises administering the dose of the bispecific antibody construct once per day for 7 days.

57. The bispecific antibody construct of claim 54 or 55, wherein the initiation cycle comprises administering the dose of the bispecific antibody construct once per day for 14 days.

58. The bispecific antibody construct of claim 54 or 55, wherein the initiation cycle comprises administering the dose of the bispecific antibody construct once every other day for 14 days.

59. The bispecific antibody construct of claim 54 or 55, wherein the initiation cycle comprises administering the dose of the bispecific antibody construct once every three days for 14 days.

60. The bispecific antibody construct of claim 54 or 55, wherein the initiation cycle comprises administering the dose of the bispecific antibody construct once every four days for 7 days or 14 days.

61. The bispecific antibody construct of any one of claims 54 to 60, wherein the initiation cycle comprises administering the bispecific antibody construct at one or more doses of about 100 μg to about 180 μg.

62. The bispecific antibody construct of any one of claims 54 to 60, wherein the initiation cycle comprises administering the bispecific antibody construct at one or more doses of about 18 μg to about 240 μg.

63. The bispecific antibody construct of any one of claims 54 to 62, wherein the dose of the bispecific antibody construct administered during the initiation cycle is the same at each interval.

64. The bispecific antibody construct of any one of claims 54 to 62, wherein the dose of the bispecific antibody construct administered during the initiation cycle increases at least once at one or more intervals during the cycle.

65. The bispecific antibody construct of claim 54 or 55, wherein the initiation cycle comprises administering the bispecific antibody construct at a first dose for one or more intervals and subsequently administering the bispecific antibody construct at a second dose for one or more intervals, wherein the second dose is greater than the first dose.

66. The bispecific antibody construct of claim 65, wherein the initiation cycle further comprises administering the bispecific antibody construct at a third dose for one or more intervals following administration of the second dose, wherein the third dose is greater than the second dose.

67. The bispecific antibody construct of claim 66, wherein the initiation cycle further comprises administering the bispecific antibody construct at a fourth dose for one or more intervals following administration of the third dose, wherein the fourth dose is greater than the third dose.

68. The bispecific antibody construct of claim 67, wherein the initiation cycle further comprises administering the bispecific antibody construct at a fifth dose for one or more intervals following administration of the fourth dose, wherein the fifth dose is greater than the fourth dose.

69. The bispecific antibody construct of claim 68, wherein the initiation cycle further comprises administering the bispecific antibody construct at a sixth dose for one or more intervals following administration of the fifth dose, wherein the sixth dose is greater than the fifth dose.

70. The bispecific antibody construct of any one of claims 65 to 69, wherein the interval is daily.

71. The bispecific antibody construct of any one of claims 65 to 70, wherein the first dose is about 18 μg to about 150 μg and the second dose is about 110 μg to about 240 μg.

72. The bispecific antibody construct of any one of claims 66 to 71, wherein the third dose is about 150 μg to about 360 μg.

73. The bispecific antibody construct of any one of claims 67 to 72, wherein the fourth dose is about 180 μg to about 480 μg.

74. The bispecific antibody construct of claim 65, wherein the first dose is about 36 μg and the second dose is about 72 μg, and wherein the interval is daily.

75. The bispecific antibody construct of claim 67, wherein the first dose is about 18 μg, the second dose is about 36 μg, the third dose is about 72 μg, and the fourth dose is about 110 μg, and wherein the interval is daily.

76. The bispecific antibody construct of claim 68, wherein the first dose is about 18 μg, the second dose is about 36 μg, the third dose is about 72 μg, the fourth dose is about 110 μg, and the fifth dose is about 160 μg, and wherein the interval is daily.

77. The bispecific antibody construct of claim 69, wherein the first dose is about 18 μg, the second dose is about 36 μg, the third dose is about 72 μg, the fourth dose is about 110 μg, the fifth dose is about 160 μg, and the sixth dose is about 240 μg, and wherein the interval is daily.

78. The bispecific antibody construct of any one of claims 54 to 77, wherein the dose of the bispecific antibody construct administered during the maintenance cycle is the same as the highest dose of the bispecific antibody construct administered during the initiation cycle.

79. The bispecific antibody construct of any one of claims 54 to 78, wherein the dose of the bispecific antibody construct administered during the maintenance cycle is about 110 μg to about 240 μg.

80. The bispecific antibody construct of any one of claims 54 to 78, wherein the dose of the bispecific antibody construct administered during the maintenance cycle is about 72 μg to about 360 μg.

81. The bispecific antibody construct of any one of claims 54 to 80, wherein the second period of time is about 14 days to about 28 days.

82. The bispecific antibody construct of any one of claims 54 to 81, wherein the maintenance cycle comprises administering the dose of the bispecific antibody construct once every 7 days for 14 days.

83. The bispecific antibody construct of any one of claims 54 to 81, wherein the maintenance cycle comprises administering the dose of the bispecific antibody construct once every 7 days for 28 days.

84. The bispecific antibody construct of any one of claims 54 to 81, wherein the maintenance cycle comprises administering the dose of the bispecific antibody construct twice every 7 days for 14 days.

85. The bispecific antibody construct of any one of claims 54 to 81, wherein the maintenance cycle comprises administering the dose of the bispecific antibody construct twice every 7 days for 28 days.

86. The bispecific antibody construct of any one of claims 54 to 85, wherein each of the doses of the bispecific antibody construct administered during the initiation cycle and/or the maintenance cycle is administered as an intravenous infusion of about 30 min to about 90 min.

87. The bispecific antibody construct of any one of claims 54 to 86, wherein the maintenance cycle is initiated the following day after completing the initiation cycle.

88. The bispecific antibody construct of any one of claims 54 to 87, wherein two or more maintenance cycles are administered to the patient.

89. The bispecific antibody construct of claim 88, wherein six to twelve maintenance cycles are administered to the patient.

90. The bispecific antibody construct of any one of claims 54 to 89, wherein the method further comprises administering to the patient a glucocorticoid prior to administration of each dose of the bispecific antibody construct during the initiation cycle and/or maintenance cycle.

91. The bispecific antibody construct of claim 90, wherein the glucocorticoid is dexamethasone.

92. The bispecific antibody construct of any one of claims 54 to 91, wherein the myeloid leukemia is acute myeloid leukemia.

93. The bispecific antibody construct of claim 92, wherein the acute myeloid leukemia is relapsed/refractory acute myeloid leukemia.

94. The bispecific antibody construct of any one of claims 54 to 91, wherein the myeloid leukemia is chronic myeloid leukemia.

95. The bispecific antibody construct of any one of claims 54 to 94, wherein the patient has previously received one or more chemotherapy regimens.

96. The bispecific antibody construct of any one of claims 54 to 95, wherein the patient has received a hematopoietic stem cell transplant.

97. The bispecific antibody construct of any one of claims 54 to 96, wherein the bispecific antibody construct comprises, in an amino to carboxyl order:
(i) a first domain that specifically binds to human CD33 comprising a first immunoglobulin heavy chain variable region (VH1) comprising a CDRH1 having the sequence of SEQ ID NO: 10, a CDRH2 having the sequence of SEQ ID NO: 13, and a CDRH3 having the sequence of SEQ ID NO: 14, and a first immunoglobulin light chain variable region (VL1) comprising a CDRL1 having the sequence of SEQ ID NO: 6, a CDRL2 having the sequence of SEQ ID NO: 8, and a CDRL3 having the sequence of SEQ ID NO: 9;
(ii) a second domain that specifically binds to human CD3 comprising a second immunoglobulin heavy chain variable region (VH2) comprising a CDRH1 having the sequence of SEQ ID NO: 38, a CDRH2 having the sequence of SEQ ID NO: 44, and a CDRH3 having the sequence of SEQ ID NO: 49, and a second immunoglobulin light chain variable region (VL2) comprising a CDRL1 having the sequence of SEQ ID NO: 32, a CDRL2 having the sequence of SEQ ID NO: 33, and a CDRL3 having the sequence of SEQ ID NO: 36; and
(iii) a third domain comprising two Fc monomers, each monomer comprising an immunoglobulin hinge region, a CH2 domain, and a CH3 domain, wherein said two monomers are fused to each other via a peptide linker.

98. The bispecific antibody construct of claim 97, wherein VH1 comprises the sequence of SEQ ID NO: 28 and VL1 comprises the sequence of SEQ ID NO: 20.

99. The bispecific antibody construct of claim 97 or 98, wherein VH2 comprises the sequence of SEQ ID NO: 61 and VL2 comprises the sequence of SEQ ID NO: 59.

100. The bispecific antibody construct of any one of claims 97 to 99, wherein the first and second binding domains are single-chain variable fragment (scFv) domains.

101. The bispecific antibody construct of any one of claims 97 to 100, wherein the first binding domain comprises the sequence of SEQ ID NO: 91.

102. The bispecific antibody construct of any one of claims 97 to 101, wherein the second binding domain comprises the sequence of SEQ ID NO: 101.

103. The bispecific antibody construct of any one of claims 97 to 102, wherein each of said Fc monomers of the third domain comprises the amino acid sequence of SEQ ID NO: 109.

104. The bispecific antibody construct of any one of claims 97 to 103, wherein the third domain comprises the amino acid sequence of SEQ ID NO: 117.

105. The bispecific antibody construct of any one of claims 97 to 104, wherein the bispecific antibody construct is a single chain antibody construct.

106. The bispecific antibody construct of claim 105, wherein the bispecific antibody construct comprises the amino acid sequence of SEQ ID NO: 125.

107. Use of a bispecific antibody construct that specifically binds to CD33 and CD3 for the manufacture of a medicament for the treatment of myeloid leukemia in a patient in need thereof, wherein the treatment comprises administering to the patient at least one initiation cycle and at least one maintenance cycle of the bispecific antibody construct,
wherein the initiation cycle comprises administering the bispecific antibody construct at one or more doses of about 18 μg to about 480 μg at an interval of 1 day to 4 days for a first period of time,
wherein the maintenance cycle comprises administering the bispecific antibody construct at a dose of about 36 μg to about 480 μg once or twice every 7 days for a second period of time, and
wherein the maintenance cycle is administered after the initiation cycle.


SEQUENCE LISTING (139 sequences)

SEQ ID NO: 1 - protein, 347 aa, Homo sapiens
DPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 2 - protein, 343 aa, Macaca fascicularis
DPRVRLEVQESVTVQEGLCVLVPCTFFHPVPYHTRNSPVHGYWFREGAIVSLDSPVATNKLDQEVQEETQGRFRLLGDPSRNNCSLSIVDARRRDNGSYFFRMEKGSTKYSYKSTQLSVHVTDLTHRPQILIPGALDPDHSKNLTCSVPWACEQGTPPIFSWMSAAPTSLGLRTTHSSVLIITPRPQDHGTNLTCQVKFPGAGVTTERTIQLNVSYASQNPRTDIFLGDGSGKQGVVQGAIGGAGVTVLLALCLCLIFFTVKTHRRKAARTAVGRIDTHPATGPTSSKHQKKSKLHGATETSGCSGTTLTVEMDEELHYASLNFHGMNPSEDTSTEYSEVRTQ

SEQ ID NO: 3 - protein, 242 aa, Homo sapiens
DPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVH

SEQ ID NO: 4 - protein, 241 aa, Macaca fascicularis
DPRVRLEVQESVTVQEGLCVLVPCTFFHPVPYHTRNSPVHGYWFREGAIVSLDSPVATNKLDQEVQEETQGRFRLLGDPSRNNCSLSIVDARRRDNGSYFFRMEKGSTKYSYKSTQLSVHVTDLTHRPQILIPGALDPDHSKNLTCSVPWACEQGTPPIFSWMSAAPTSLGLRTTHSSVLIITPRPQDHGTNLTCQVKFPGAGVTTERTIQLNVSYASQNPRTDIFLGDGSGKQGVVQGAI

SEQ ID NO: 5 - protein, 17 aa, Artificial Sequence
  Synthetic polypeptide
KSSQSVLDSSKNKNSLA

SEQ ID NO: 6 - protein, 17 aa, Artificial Sequence
  Synthetic polypeptide
KSSQSVLDSSTNKNSLA

SEQ ID NO: 7 - protein, 17 aa, Artificial Sequence
  Synthetic polypeptide
KSSQSVLDSSNNKNSLA

SEQ ID NO: 8 - protein, 7 aa, Artificial Sequence
  Synthetic polypeptide
WASTRES

SEQ ID NO: 9 - protein, 9 aa, Artificial Sequence
  Synthetic polypeptide
QQSAHFPIT

SEQ ID NO: 10 - protein, 5 aa, Artificial Sequence
  Synthetic polypeptide
NYGMN

SEQ ID NO: 11 - protein, 17 aa, Artificial Sequence
  Synthetic polypeptide
WINTYTGEPTYADDFKG

SEQ ID NO: 12 - protein, 17 aa, Artificial Sequence
  Synthetic polypeptide
WINTYTGETNYADKFQG

SEQ ID NO: 13 - protein, 17 aa, Artificial Sequence
  Synthetic polypeptide
WINTYTGEPTYADKFQG

SEQ ID NO: 14 - protein, 13 aa, Artificial Sequence
  Synthetic polypeptide
WSWSDGYYVYFDY

SEQ ID NO: 15 - protein, 113 aa, Artificial Sequence
  Synthetic polypeptide
DIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIK

SEQ ID NO: 16 - protein, 113 aa, Artificial Sequence
  Synthetic polypeptide
DIVMTQSPDSMTVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLDIK

SEQ ID NO: 17 - protein, 113 aa, Artificial Sequence
  Synthetic polypeptide
DIVMTQSPDSLSVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLEIK

SEQ ID NO: 18 - protein, 113 aa, Artificial Sequence
  Synthetic polypeptide
DIVMTQSPDSLTVSLGERTTINCKSSQSVLDSSNNKNSLAWYQQKPGQPPKLLLSWASTRESGIPDRFSGSGSGTDFTLTIDGLQPEDSATYYCQQSAHFPITFGQGTRLEIK

SEQ ID NO: 19 - protein, 113 aa, Artificial Sequence
  Synthetic polypeptide
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 20 - protein, 114 aa, Artificial Sequence
  Synthetic polypeptide
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKS

SEQ ID NO: 21 - protein, 122 aa, Artificial Sequence
  Synthetic polypeptide
QVQLVQSGAEVKKPGESVKVSCKASGYTFTNYGMNWVRQAPGQGLEWMGWINTYTGEPTYADDFKGRVTMSSDTSTSTAYLEINSLRSDDTAIYYCARWSWSDGYYVYFDYWGQGTTVTVSS

SEQ ID NO: 22 - protein, 122 aa, Artificial Sequence
  Synthetic polypeptide
QVQLVQSGAEVKKPGASVKVSCKASGYTFTNYGMNWVKQAPGQGLKWMGWINTYTGEPTYADDFKGRVTMTSDTSTSTAYLELHNLRSDDTAVYYCARWSWSDGYYVYFDYWGQGTTVTVSS

SEQ ID NO: 23 - protein, 122 aa, Artificial Sequence
  Synthetic polypeptide
QVQLVQSGAEVKKPGESVKVSCKASGYTFTNYGMNWVKQAPGQGLKWMGWINTYTGEPTYADDFKGRVTMTTDTSTSTAYMEIRNLRNDDTAVYYCARWSWSDGYYVYFDYWGQGTTVTVSS

SEQ ID NO: 24 - protein, 122 aa, Artificial Sequence
  Synthetic polypeptide
QVQLVQSGAEVKKPGESVKVSCKASGYTFTNYGMNWVKQAPGQGLKWMGWINTYTGEPTYADDFKGRVTMTSDTSTSTAYMEISSLRSDDTAVYYCARWSWSDGYYVYFDYWGQGTTVTVSS

SEQ ID NO: 25 - protein, 122 aa, Artificial Sequence
  Synthetic polypeptide
QVQLVQSGAEVKKPGESVKVSCKASGYTFTNYGMNWVKQAPGQGLEWMGWINTYTGETNYADKFQGRVTFTSDTSTSTAYMELRNLKSDDTAVYYCARWSWSDGYYVYFDYWGQGTTVTVSS

SEQ ID NO: 26 - protein, 122 aa, Artificial Sequence
  Synthetic polypeptide
QVQLVQSGAEVKKPGESVKVSCKASGYTFTNYGMNWVKQAPGQGLEWMGWINTYTGEPTYADKFQGRVTMTTDTSTSTAYMEIRNLRSDDTAVYYCARWSWSDGYYVYFDYWGQGTTVTVSS

SEQ ID NO: 27 - protein, 122 aa, Artificial Sequence
  Synthetic polypeptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSS

SEQ ID NO: 28 - protein, 122 aa, Artificial Sequence
  Synthetic polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSS

SEQ ID NO: 29 - protein, 105 aa, Homo sapiens
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 30 - protein, 14 aa, Artificial Sequence
  Synthetic polypeptide
GSSTGAVTSGYYPN

SEQ ID NO: 31 - protein, 14 aa, Artificial Sequence
  Synthetic polypeptide
RSSTGAVTSGYYPN

SEQ ID NO: 32 - protein, 14 aa, Artificial Sequence
  Synthetic polypeptide
GSSTGAVTSGNYPN

SEQ ID NO: 33 - protein, 7 aa, Artificial Sequence
  Synthetic polypeptide
GTKFLAP

SEQ ID NO: 34 - protein, 7 aa, Artificial Sequence
  Synthetic polypeptide
ATDMRPS

SEQ ID NO: 35 - protein, 9 aa, Artificial Sequence
  Synthetic polypeptide
ALWYSNRWV

SEQ ID NO: 36 - protein, 9 aa, Artificial Sequence
  Synthetic polypeptide
VLWYSNRWV

SEQ ID NO: 37 - protein, 5 aa, Artificial Sequence
  Synthetic polypeptide
IYAMN

SEQ ID NO: 38 - protein, 5 aa, Artificial Sequence
  Synthetic polypeptide
KYAMN

SEQ ID NO: 39 - protein, 5 aa, Artificial Sequence
  Synthetic polypeptide
SYAMN

SEQ ID NO: 40 - protein, 5 aa, Artificial Sequence
  Synthetic polypeptide
RYAMN

SEQ ID NO: 41 - protein, 5 aa, Artificial Sequence
  Synthetic polypeptide
VYAMN

SEQ ID NO: 42 - protein, 5 aa, Artificial Sequence
  Synthetic polypeptide
GYAMN

SEQ ID NO: 43 - protein, 19 aa, Artificial Sequence
  Synthetic polypeptide
RIRSKYNNYATYYADSVKS

SEQ ID NO: 44 - protein, 19 aa, Artificial Sequence
  Synthetic polypeptide
RIRSKYNNYATYYADSVKD

SEQ ID NO: 45 - protein, 19 aa, Artificial Sequence
  Synthetic polypeptide
RIRSKYNNYATYYADSVKG

SEQ ID NO: 46 - protein, 19 aa, Artificial Sequence
  Synthetic polypeptide
RIRSKYNNYATYYADSVKK

SEQ ID NO: 47 - protein, 19 aa, Artificial Sequence
  Synthetic polypeptide
RIRSKYNNYATYYADSVKE

SEQ ID NO: 48 - protein, 14 aa, Artificial Sequence
  Synthetic polypeptide
HGNFGNSYVSFFAY

SEQ ID NO: 49 - protein, 14 aa, Artificial Sequence
  Synthetic polypeptide
HGNFGNSYISYWAY

SEQ ID NO: 50 - protein, 14 aa, Artificial Sequence
  Synthetic polypeptide
HGNFGNSYLSFWAY

SEQ ID NO: 51 - protein, 14 aa, Artificial Sequence
  Synthetic polypeptide
HGNFGNSYLSYFAY

SEQ ID NO: 52 - protein, 14 aa, Artificial Sequence
  Synthetic polypeptide
HGNFGNSYLSWWAY

SEQ ID NO: 53 - protein, 14 aa, Artificial Sequence
  Synthetic polypeptide
HGNFGNSYTSYYAY

SEQ ID NO: 54 - protein, 14 aa, Artificial Sequence
  Synthetic polypeptide
HRNFGNSYLSWFAY

SEQ ID NO: 55 - protein, 14 aa, Artificial Sequence
  Synthetic polypeptide
HGNFGNSYISWWAY

SEQ ID NO: 56 - protein, 14 aa, Artificial Sequence
  Synthetic polypeptide
HGNFGNSYVSWWAY

SEQ ID NO: 57 - protein, 109 aa, Artificial Sequence
  Synthetic polypeptide
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 58 - protein, 109 aa, Artificial Sequence
  Synthetic polypeptide
QTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 59 - protein, 109 aa, Artificial Sequence
  Synthetic polypeptide
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 60 - protein, 125 aa, Artificial Sequence
  Synthetic polypeptide
EVQLVESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSS

SEQ ID NO: 61 - protein, 125 aa, Artificial Sequence
  Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 62 - protein, 125 aa, Artificial Sequence
  Synthetic polypeptide
EVQLVESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSS

SEQ ID NO: 63 - protein, 125 aa, Artificial Sequence
  Synthetic polypeptide
EVQLVESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSS

SEQ ID NO: 64 - protein, 125 aa, Artificial Sequence
  Synthetic polypeptide
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSS

SEQ ID NO: 65 - protein, 125 aa, Artificial Sequence
  Synthetic polypeptide
EVQLVESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSS

SEQ ID NO: 66 - protein, 125 aa, Artificial Sequence
  Synthetic polypeptide
EVQLVESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSS

SEQ ID NO: 67 - protein, 125 aa, Artificial Sequence
  Synthetic polypeptide
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSS

SEQ ID NO: 68 - protein, 125 aa, Artificial Sequence
  Synthetic polypeptide
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSS

SEQ ID NO: 69 - protein, 4 aa, Artificial Sequence
  Synthetic polypeptide
GGGG

SEQ ID NO: 70 - protein, 5 aa, Artificial Sequence
  Synthetic polypeptide
GGGGS

SEQ ID NO: 71 - protein, 10 aa, Artificial Sequence
  Synthetic polypeptide
GGGGSGGGGS

SEQ ID NO: 72 - protein, 15 aa, Artificial Sequence
  Synthetic polypeptide
GGGGSGGGGSGGGGS

SEQ ID NO: 73 - protein, 20 aa, Artificial Sequence
  Synthetic polypeptide
GGGGSGGGGSGGGGSGGGGS

SEQ ID NO: 74 - protein, 25 aa, Artificial Sequence
  Synthetic polypeptide
GGGGSGGGGSGGGGSGGGGSGGGGS

SEQ ID NO: 75 - protein, 30 aa, Artificial Sequence
  Synthetic polypeptide
GGGGSGGGGSGGGGSGGGGSGGGGSGGGGS

SEQ ID NO: 76 - protein, 35 aa, Artificial Sequence
  Synthetic polypeptide
GGGGSGGGGSGGGGSGGGGSGGGGSGGGGSGGGGS

SEQ ID NO: 77 - protein, 40 aa, Artificial Sequence
  Synthetic polypeptide
GGGGSGGGGSGGGGSGGGGSGGGGSGGGGSGGGGSGGGGS

SEQ ID NO: 78 - protein, 6 aa, Artificial Sequence
  Synthetic polypeptide
PGGGGS

SEQ ID NO: 79 - protein, 6 aa, Artificial Sequence
  Synthetic polypeptide
PGGDGS

SEQ ID NO: 80 - protein, 6 aa, Artificial Sequence
  Synthetic polypeptide
SGGGGS

SEQ ID NO: 81 - protein, 9 aa, Artificial Sequence
  Synthetic polypeptide
GGGGSGGGS

SEQ ID NO: 82 - protein, 5 aa, Artificial Sequence
  Synthetic polypeptide
GGGGQ

SEQ ID NO: 83 - protein, 250 aa, Artificial Sequence
  Synthetic polypeptide
QVQLVQSGAEVKKPGESVKVSCKASGYTFTNYGMNWVRQAPGQGLEWMGWINTYTGEPTYADDFKGRVTMSSDTSTSTAYLEINSLRSDDTAIYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIK

SEQ ID NO: 84 - protein, 250 aa, Artificial Sequence
  Synthetic polypeptide
QVQLVQSGAEVKKPGASVKVSCKASGYTFTNYGMNWVKQAPGQGLKWMGWINTYTGEPTYADDFKGRVTMTSDTSTSTAYLELHNLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIK

SEQ ID NO: 85 - protein, 250 aa, Artificial Sequence
  Synthetic polypeptide
QVQLVQSGAEVKKPGESVKVSCKASGYTFTNYGMNWVKQAPGQGLKWMGWINTYTGEPTYADDFKGRVTMTTDTSTSTAYMEIRNLRNDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIK

SEQ ID NO: 86 - protein, 250 aa, Artificial Sequence
  Synthetic polypeptide
QVQLVQSGAEVKKPGESVKVSCKASGYTFTNYGMNWVKQAPGQGLKWMGWINTYTGEPTYADDFKGRVTMTSDTSTSTAYMEISSLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIK

SEQ ID NO: 87 - protein, 250 aa, Artificial Sequence
  Synthetic polypeptide
QVQLVQSGAEVKKPGESVKVSCKASGYTFTNYGMNWVKQAPGQGLEWMGWINTYTGETNYADKFQGRVTFTSDTSTSTAYMELRNLKSDDTAVYYCARWSWSDGYYVYFDYWGQGTTVTVSSGGGGSGGGGSGGGGSDIVMTQSPDSMTVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLDIK

SEQ ID NO: 88 - protein, 250 aa, Artificial Sequence
  Synthetic polypeptide
QVQLVQSGAEVKKPGESVKVSCKASGYTFTNYGMNWVKQAPGQGLEWMGWINTYTGETNYADKFQGRVTFTSDTSTSTAYMELRNLKSDDTAVYYCARWSWSDGYYVYFDYWGQGTTVTVSSGGGGSGGGGSGGGGSDIVMTQSPDSLSVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLEIK

SEQ ID NO: 89 - protein, 250 aa, Artificial Sequence
  Synthetic polypeptide
QVQLVQSGAEVKKPGESVKVSCKASGYTFTNYGMNWVKQAPGQGLEWMGWINTYTGEPTYADKFQGRVTMTTDTSTSTAYMEIRNLRSDDTAVYYCARWSWSDGYYVYFDYWGQGTTVTVSSGGGGSGGGGSGGGGSDIVMTQSPDSLTVSLGERTTINCKSSQSVLDSSNNKNSLAWYQQKPGQPPKLLLSWASTRESGIPDRFSGSGSGTDFTLTIDGLQPEDSATYYCQQSAHFPITFGQGTRLEIK

SEQ ID NO: 90 - protein, 250 aa, Artificial Sequence
  Synthetic polypeptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 91 - protein, 250 aa, Artificial Sequence
  Synthetic polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIK

SEQ ID NO: 92 - protein, 249 aa, Artificial Sequence
  Synthetic polypeptide
EVQLVESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 93 - protein, 249 aa, Artificial Sequence
  Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 94 - protein, 249 aa, Artificial Sequence
  Synthetic polypeptide
EVQLVESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 95 - protein, 249 aa, Artificial Sequence
  Synthetic polypeptide
EVQLVESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 96 - protein, 249 aa, Artificial Sequence
  Synthetic polypeptide
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSSGGGGSGGGGSGGGGSQTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 97 - protein, 249 aa, Artificial Sequence
  Synthetic polypeptide
EVQLVESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 98 - protein, 249 aa, Artificial Sequence
  Synthetic polypeptide
EVQLVESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSSGGGGSGGGGSGGGGSQTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 99 - protein, 249 aa, Artificial Sequence
  Synthetic polypeptide
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 100 - protein, 249 aa, Artificial Sequence
  Synthetic polypeptide
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 101 - protein, 249 aa, Artificial Sequence
  Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 102 - protein, 10 aa, Artificial Sequence
  Synthetic polypeptide
DKTHTCPPCP

SEQ ID NO: 103 - protein, 15 aa, Artificial Sequence
  Synthetic polypeptide
EPKSCDKTHTCPPCP

SEQ ID NO: 104 - protein, 12 aa, Artificial Sequence
  Synthetic polypeptide
ERKCCVECPPCP

SEQ ID NO: 105 - protein, 16 aa, Artificial Sequence
  Synthetic polypeptide
ELKTPLDTTHTCPRCP

SEQ ID NO: 106 - protein, 15 aa, Artificial Sequence
  Synthetic polypeptide
EPKSCDTPPPCPRCP

SEQ ID NO: 107 - protein, 17 aa, Artificial Sequence
  Synthetic polypeptide
ELKTPLGDTTHTCPRCP

SEQ ID NO: 108 - protein, 12 aa, Artificial Sequence
  Synthetic polypeptide
ESKYGPPCPSCP

SEQ ID NO: 109 - protein, 227 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 225 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 111 - protein, 227 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 225 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 113 - protein, 227 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 225 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 115 - protein, 227 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 225 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 117 - protein, 484 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 480 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 119 - protein, 484 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 480 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 121 - protein, 484 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 480 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 123 - protein, 484 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 480 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 125 - protein, 993 aa, Artificial Sequence
  Synthetic polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 989 aa, Artificial Sequence
  Synthetic polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 127 - protein, 993 aa, Artificial Sequence
  Synthetic polypeptide
QVQLVQSGAEVKKPGESVKVSCKASGYTFTNYGMNWVRQAPGQGLEWMGWINTYTGEPTYADDFKGRVTMSSDTSTSTAYLEINSLRSDDTAIYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 993 aa, Artificial Sequence
  Synthetic polypeptide
QVQLVQSGAEVKKPGASVKVSCKASGYTFTNYGMNWVKQAPGQGLKWMGWINTYTGEPTYADDFKGRVTMTSDTSTSTAYLELHNLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 993 aa, Artificial Sequence
  Synthetic polypeptide
QVQLVQSGAEVKKPGESVKVSCKASGYTFTNYGMNWVKQAPGQGLKWMGWINTYTGEPTYADDFKGRVTMTTDTSTSTAYMEIRNLRNDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 993 aa, Artificial Sequence
  Synthetic polypeptide
QVQLVQSGAEVKKPGESVKVSCKASGYTFTNYGMNWVKQAPGQGLKWMGWINTYTGEPTYADDFKGRVTMTSDTSTSTAYMEISSLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 993 aa, Artificial Sequence
  Synthetic polypeptide
QVQLVQSGAEVKKPGESVKVSCKASGYTFTNYGMNWVKQAPGQGLEWMGWINTYTGETNYADKFQGRVTFTSDTSTSTAYMELRNLKSDDTAVYYCARWSWSDGYYVYFDYWGQGTTVTVSSGGGGSGGGGSGGGGSDIVMTQSPDSMTVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLDIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 993 aa, Artificial Sequence
  Synthetic polypeptide
QVQLVQSGAEVKKPGESVKVSCKASGYTFTNYGMNWVKQAPGQGLEWMGWINTYTGETNYADKFQGRVTFTSDTSTSTAYMELRNLKSDDTAVYYCARWSWSDGYYVYFDYWGQGTTVTVSSGGGGSGGGGSGGGGSDIVMTQSPDSLSVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 993 aa, Artificial Sequence
  Synthetic polypeptide
QVQLVQSGAEVKKPGESVKVSCKASGYTFTNYGMNWVKQAPGQGLEWMGWINTYTGEPTYADKFQGRVTMTTDTSTSTAYMEIRNLRSDDTAVYYCARWSWSDGYYVYFDYWGQGTTVTVSSGGGGSGGGGSGGGGSDIVMTQSPDSLTVSLGERTTINCKSSQSVLDSSNNKNSLAWYQQKPGQPPKLLLSWASTRESGIPDRFSGSGSGTDFTLTIDGLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 993 aa, Artificial Sequence
  Synthetic polypeptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 989 aa, Artificial Sequence
  Synthetic polypeptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 136 - protein, 993 aa, Artificial Sequence
  Synthetic polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 993 aa, Artificial Sequence
  Synthetic polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 993 aa, Artificial Sequence
  Synthetic polypeptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 993 aa, Artificial Sequence
  Synthetic polypeptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK